SEQUENCE LISTING (32 sequences)

SEQ ID NO: 1 - DNA, 14 bp, Artificial Sequence
  Synthetic oligonucleotide
cutagcactggccu

SEQ ID NO: 2 - DNA, 14 bp, Artificial Sequence
  Synthetic oligonucleotide
cttagcactggcct

SEQ ID NO: 3 - DNA, 18 bp, Artificial Sequence
  Synthetic oligonucleotide
cugctagcctctggatuu

SEQ ID NO: 4 - DNA, 18 bp, Artificial Sequence
  Synthetic oligonucleotide
ctgctagcctctggattt

SEQ ID NO: 5 - DNA, 16 bp, Artificial Sequence
  Synthetic oligonucleotide
tcttagcactggcctt

SEQ ID NO: 6 - DNA, 17 bp, Artificial Sequence
  Synthetic oligonucleotide
ctcttagcactggcctt

SEQ ID NO: 7 - RNA, 14 nt, Artificial Sequence
  Synthetic oligonucleotide
aggccagugcuaag

SEQ ID NO: 8 - RNA, 20 nt, Artificial Sequence
  Synthetic oligonucleotide
ucaaggccagugcuaagagu

SEQ ID NO: 9 - DNA, 15 bp, Artificial Sequence
  Synthetic oligonucleotide
tgctggcagacttac

SEQ ID NO: 10 - DNA, 15 bp, Artificial Sequence
  Synthetic oligonucleotide
cataatgctggcaga

SEQ ID NO: 11 - DNA, 15 bp, Artificial Sequence
  Synthetic oligonucleotide
tcataatgctggcag

SEQ ID NO: 12 - DNA, 15 bp, Artificial Sequence
  Synthetic oligonucleotide
ttcataatgctggca

SEQ ID NO: 13 - DNA, 15 bp, Artificial Sequence
  Synthetic oligonucleotide
tttcataatgctggc

SEQ ID NO: 14 - DNA, 20 bp, Artificial Sequence
  Synthetic oligonucleotide
attcactttcataatgctgg

SEQ ID NO: 15 - DNA, 18 bp, Artificial Sequence
  Synthetic oligonucleotide
tcactttcataatgctgg

SEQ ID NO: 16 - DNA, 15 bp, Artificial Sequence
  Synthetic oligonucleotide
ctttcataatgctgg

SEQ ID NO: 17 - DNA, 12 bp, Artificial Sequence
  Synthetic oligonucleotide
tcataatgctgg

SEQ ID NO: 18 - DNA, 15 bp, Artificial Sequence
  Synthetic oligonucleotide
actttcataatgctg

SEQ ID NO: 19 - DNA, 12 bp, Artificial Sequence
  Synthetic oligonucleotide
ttcataatgctg

SEQ ID NO: 20 - DNA, 15 bp, Artificial Sequence
  Synthetic oligonucleotide
cactttcataatgct

SEQ ID NO: 21 - DNA, 12 bp, Artificial Sequence
  Synthetic oligonucleotide
tttcataatgct

SEQ ID NO: 22 - DNA, 15 bp, Artificial Sequence
  Synthetic oligonucleotide
tcactttcataatgc

SEQ ID NO: 23 - DNA, 12 bp, Artificial Sequence
  Synthetic oligonucleotide
ctttcataatgc

SEQ ID NO: 24 - DNA, 15 bp, Artificial Sequence
  Synthetic oligonucleotide
ttcactttcataatg

SEQ ID NO: 25 - DNA, 12 bp, Artificial Sequence
  Synthetic oligonucleotide
actttcataatg

SEQ ID NO: 26 - DNA, 15 bp, Artificial Sequence
  Synthetic oligonucleotide
attcactttcataat

SEQ ID NO: 27 - DNA, 12 bp, Artificial Sequence
  Synthetic oligonucleotide
cactttcataat

SEQ ID NO: 28 - DNA, 15 bp, Artificial Sequence
  Synthetic oligonucleotide
gattcactttcataa

SEQ ID NO: 29 - DNA, 12 bp, Artificial Sequence
  Synthetic oligonucleotide
tcactttcataa

SEQ ID NO: 30 - DNA, 12 bp, Artificial Sequence
  Synthetic oligonucleotide
ttcactttcata

SEQ ID NO: 31 - DNA, 12 bp, Artificial Sequence
  Synthetic oligonucleotide
attcactttcat

SEQ ID NO: 32 - DNA, 15 bp, Artificial Sequence
  Synthetic oligonucleotide
agtaagattcacttt

(19) United States
(12) Patent Application Publication
(10) Pub. No.: US 2012/0021515 A1
(43) Pub. Date: Jan. 26, 2012

(54) OLIGOMERIC COMPOUNDS AND METHODS

(76) Inventors: Eric E. Swayze, Encinitas, CA (US); Andrew M. Siwkowski, Carlsbad, CA (US); Balkrishen Bhat, Carlsbad, CA (US); Thazha P. Prakash, Carlsbad, CA (US); Charles Allerson, San Diego, CA (US); Punit P. Seth, Carlsbad, CA (US)

(21) Appl. No.: 13/148,288

(22) PCT Filed: Feb. 5, 2010
(86) PCT No.: PCT/US10/23397

§ 371 Date: Oct. 7, 2011

Related U.S. Application Data
(60) Provisional application No. 61/150,710, filed on Feb. 6, 2009.

(51) Int. Cl. C12N 5/071; C07H 21/00; C07H 19/02

(52) U.S. Cl. 435/375; 536/22.1; 536/26.5; 536/26.1

(57) ABSTRACT

The present invention provides oligomeric compounds and uses thereof. In certain embodiments, such oligomeric compounds are useful as antisense compounds. Certain such antisense compounds are useful as RNase H antisense compounds, as RNAi compounds, and/or as modulators of splicing.

### FIELD OF THE INVENTION

The present invention provides compounds and methods for modulating nucleic acids and proteins.

### SEQUENCE LISTING

The present application is being filed along with a Sequence Listing in electronic format. The Sequence Listing is provided as a file entitled CORE0082WOSEQ.txt, created on Feb. 5, 2010 which is 8 Kb in size. The information in the electronic format of the sequence listing is incorporated herein by reference in its entirety.

### BACKGROUND OF THE INVENTION

Antisense technology is an effective means for reducing the expression of one or more specific gene products and can therefore prove to be uniquely useful in a number of therapeutic, diagnostic, and research applications. Chemically modified nucleosides are routinely used for incorporation into antisense sequences to enhance one or more properties such as for example affinity and nuclease resistance. One such group of chemically modified nucleosides includes tetrahydropyran nucleoside analogs wherein the furanose ring is replaced with a tetrahydropyran ring.

The synthesis of various tetrahydropyran nucleoside analogs has been reported in the literature, see for example: Verheggen et al., J. Med. Chem., 1995, 38, 826-835; Altmann et al., Chimia, 1996, 50, 168-176; Herdewijn et al., Bioorganic & Medicinal Chemistry Letters, 1996, 6 (13), 1457-1460; Verheggen et al., Nucleosides & Nucleotides, 1996, 15(1-3), 325-335; Ostrowski et al., J. Med. Chem., 1998, 41, 4343-4353; Allart et al., Tetrahedron., 1999, 55, 6527-6546; Wouters et al., Bioorganic & Medicinal Chemistry Letters, 1999, 9, 1563-1566; Brown, et al., Drug Development Res., 2000, 49, 253-259; published PCT application: WO 93/25565; WO 02/18406; and WO 05/049582; U.S. Pat. Nos. 5,314,893; 5,607,922; and 6,455,507.

Various tetrahydropyran nucleoside analogs have been described as monomers and have also been incorporated into oligomeric compounds (see for example: Published PCT application, WO 93/25565, published Dec. 23, 1993; Augustyns et al. Nucleic Acids Res., 1993, 21(20), 4670-4676; Verheggen et al., J. Med. Chem., 1993, 36, 2033-2040; Van Aerschol et al., Angew. Chem. Int. Ed. Engl., 1995, 34(12), 1338-1339; Anderson et al., Tetrahedron Letters, 1996, 37(45), 8147-8150; Herdewijn et al., Liebigs Ann., 1996, 1337-1348; De Bouvere et al., Liebigs Ann./Recueil, 1997, 1453-1461; 1513-1520; Hendrix et al., Chem. Eur. J., 1997, 3(1), 110-120; Hendrix et al., Chem. Eur. J., 1997, 3(9), 1513-1520; Hossain et al, J. Org. Chem., 1998, 63, 1574-1582; Allart et al., Chem. Eur. J., 1999, 5(8), 2424-2431; Boudou et al., Nucleic Acids Res., 1999, 27(6), 1450-1456; Kozlov et al., J. Am. Chem. Soc., 1999, 121, 1108-1109; Kozlov et al., J. Am. Chem. Soc., 1999, 121, 2653-2656; Kozlov et al., J. Am. Chem. Soc., 1999, 121, 5856-5859; Pochet et al., Nucleosides & Nucleotides, 1999, 18 (4&5), 1015-1017; Vastmans et al., Collection Symposium Series, 1999, 2, 156-160; Froeyen et al., Helvetica Chimica Acta, 2000, 83, 2153-2182; Kozlov et al., Chem. Eur. J., 2000, 6(1), 151-155; Atkins et al., Parmazie, 2000, 55(8), 615-617; Lescrinier et al., Chemistry & Biology, 2000, 7, 719-731; Lescrinier et al., Helvetica Chimica Acta, 2000, 83, 1291-1310; Wang et al., J. Am. Chem., 2000, 122, 8595-8602; US Patent Application US 2004/0033967; Published US Patent Application US 2008/0038745; Published and Issued U.S. Pat. No. 7,276,592). DNA analogs have also been reviewed in an article (see: Leumann, J. C, Bioorganic & Medicinal Chemistry, 2002, 10, 841-854) which included a general discussion of tetrahydropyran nucleoside analogs (under the name: hexitol nucleic acid family).

### SUMMARY OF THE INVENTION

In certain embodiments, the present invention provides compounds comprising an oligomeric compound consisting of 12 to 30 linked monomers, wherein the oligomeric compound comprises at least 4 regions, wherein each monomer within each region comprises the same type of sugar moiety and wherein the sugar moieties monomers of adjacent regions are different from one another; and wherein:

at least one region comprises 2-20 linked monomers and each of the other regions independently comprises 1-20 linked monomers; and wherein

at least one region is a tetrahydropyran region, wherein each tetrahydropyran region independently comprises one or more tetrahydropyran nucleoside analog of Formula I:

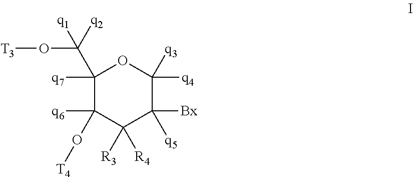

wherein independently for each of said tetrahydropyran nucleoside analogs of Formula I:

Bx is a heterocyclic base moiety;

T₃ and T₄ are each, independently, an internucleoside linking group linking the tetrahydropyran nucleoside analog to the oligomeric compound or one of T₃ and T₄ is an internucleoside linking group linking the tetrahydropyran nucleoside analog to the oligomeric compound and the other of T₃ and T₄ is H, a hydroxyl protecting group, a linked conjugate group or a 5′ or 3′-terminal group;

q₁, q₂, q₃, q₄, q₅, q₆ and q₇ are each independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;

R₃ and R₄ are each independently, H, hydroxyl, halogen, C₁-C₆ alkyl, substituted C₁-C₆ alkyl, C₁-C₆ alkoxy or substituted C₁-C₆ alkoxy;

each substituted group comprises one or more optionally protected substituent groups independently selected from halogen, OJ₁, NJ₁J₂, SJ₁, N₃, OC(═X)Jᵢ, OC(═X)NJ₁J₂, NJ₃C(═X)NJ₁J₂ and CN, wherein X is O, S or NJ₁ and each J₁, J₂ and J₃ is, independently, H or C₁-C₆ alkyl; and

wherein the remaining regions are non-tetrahydropyran regions, wherein the monomers of each non-tetrahydropyran region are independently modified or unmodified nucleosides or nucleoside analogs other than tetrahydropyran nucleoside analogs.

In certain embodiments, such compounds comprises at least 5, 6, 7, 8, 9, 10, 12, 13, 14, 15, 16, 18, 19, 20, 21, 22, 23, 24, 25, 26, 27, 28, or 29 regions.

In certain embodiments, such compounds comprise at least 2, 3, 4, 5, 6, 7, 8, 9, 10, 11, 12, 13, 14, or 15 regions comprises 2 or more linked monomers.

In certain embodiments, such compounds have at least 2, 3, 4, 5, 6, 7, 8, 9, 10, 12, 13, 14, 15, 16, 18, 19, 20, 21, 22, 23, 24, 25, 26, 27, 28, or 29 regions of tetrahydropyran monomers of Formula I.

In embodiments comprising more than one tetrahydropyran monomers of Formula I, such tetrahydropyran monomers may be the same or different from one another.

In certain embodiments, the non-tetrahydropyran monomers may be modified or unmodified nucleosides.

In certain embodiments, oligomeric compounds have a motif:

5′-A(-L-B-L-A)ₙ(-L-B)ₙₘ-3′
wherein one of each A or each B is a tetrahydropyran region and the other of each A or B is a non-tetrahydropyran region;
each L is an internucleoside linking group, nn is 0 or 1; and
n is from 4 to about 12.
The compound of any one of claims 1-47 having a motif:

T₁-(Nu₁)ₙ₁-(Nu₂)ₙ₂-(Nu₃)ₙ₃-(Nu₄)ₙ₄-(Nu₅)ₙ₅-T₂, wherein:
Nu₁, Nu₃, and Nu₅ are each independently tetrahydropyran nucleoside analogs of Formula I;
Nu₂ and Nu₄ are each independently modified or unmodified nucleosides or nucleoside analogs other than tetrahydropyran nucleoside analogs;
each of n1 and n5 is, independently from 0 to 3;
the sum of n2 plus n4 is between 10 and 25;
n3 is from 0 and 5; and
each T₁ and T₂ is, independently, H, a hydroxyl protecting group, an optionally
linked conjugate group or a capping group.

In certain embodiments, oligomeric compounded have a motif:

T₁-(Nu₁)ₙ₁-(Nu₂)ₙ₂-(Nu₃)ₙ₃-(Nu₄)ₙ₄-(Nu₅)ₙ₅-T₂, wherein:
Nu₁, Nu₃, and Nu₅ are each independently modified or unmodified nucleosides or nucleoside analogs other than tetrahydropyran nucleoside analogs;
Nu₂ and Nu₄ are each independently tetrahydropyran nucleoside analogs of Formula I;
each of n1 and n5 is, independently from 0 to 3;
the sum of n2 plus n4 is between 10 and 25;
n3 is from 0 and 5; and
each T₁ and T₂ is, independently, H, a hydroxyl protecting group, an optionally linked conjugate group or a capping group.

In certain embodiments, oligomeric compounds have at least one region having a motif selected from:
Nu₁ Nu₁ Nu₂ Nu₂ Nu₁ Nu₁;
Nu₁ Nu₂ Nu₂ Nu₁ Nu₂ Nu₂;
Nu₁ Nu₁ Nu₂ Nu₁ Nu₁ Nu₂;
Nu₁ Nu₂ Nu₂ Nu₁ Nu₂ Nu₁ Nu₁ Nu₂ Nu₂;
Nu₁ Nu₂ Nu₁ Nu₂ Nu₁ Nu₁;
Nu₁ Nu₁ Nu₂ Nu₁ Nu₂ Nu₁ Nu₂;
Nu₁ Nu₂ Nu₁ Nu₂ Nu₁ Nu₁;
Nu₁ Nu₂ Nu₂ Nu₁ Nu₁ Nu₂ Nu₂ Nu₁ Nu₂ Nu₁ Nu₂ Nu₁ Nu₁;
Nu₂ Nu₁ Nu₂ Nu₁ Nu₁ Nu₁ Nu₂Nu₂Nu₁ Nu₂Nu₁ Nu₂Nu₁ Nu₁; and
Nu₁ Nu₂Nu₁ Nu₂ Nu₂ Nu₁ Nu₁ Nu₂Nu₂Nu₁ Nu₂ Nu₁ Nu₂ Nu₁ Nu₁; and
wherein one of Nu₁ and Nu₂ is a tetrahydropyran nucleoside analog of Formula I and the other of Nu₁ and Nu₂ is a non-tetrahydropyran nucleoside or nucleoside analog.
In certain embodiments, each tetrahydropyran nucleoside analog of Formula I has the configuration of Formula II:

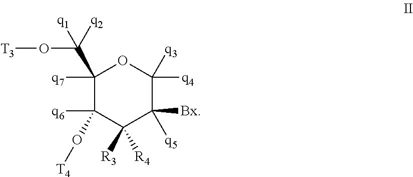

In certain embodiments, at least one tetrahydropyran nucleoside analog has Formula III:

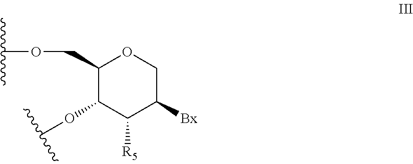

wherein:
Bx is a heterocyclic base moiety; and
R₅ is H, OCH₃ or F.

In certain embodiments, compounds of the invention are antisense compounds. In certain such embodiments, at least a portion of the nucleobase sequence of the oligomeric compound is complementary to a portion of a target nucleic acid, wherein the target nucleic acid is selected from: a target mRNA, a target pre-mRNA, a target microRNA, and a target non-coding RNA.

In certain embodiments, the invention provides methods of modulating the amount or activity of a target nucleic acid in a cell comprising contacting the cell with a compound of the present invention and thereby amount or activity of the target nucleic acid in the cell.

In certain embodiments, the invention provides compounds comprising an oligomeric compound consisting of 12 to 30 linked monomers, wherein the oligomeric compound comprises at least one monomer comprising a tetrahydropyran nucleoside analog of Formula I:

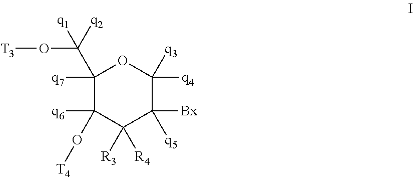

wherein independently for each of said tetrahydropyran nucleoside analogs of Formula I:
Bx is a heterocyclic base moiety;
T₃ and T₄ are each, independently, an internucleoside linking group linking the tetrahydropyran nucleoside analog to the oligomeric compound or one of T₃ and T₄ is an internucleoside linking group linking the tetrahydropyran nucleoside analog to the oligomeric compound and the other of T₃ and T₄ is H, a hydroxyl protecting group, a linked conjugate group or a 5′ or 3′-terminal group;
q₁, q₂, q₃, q₄, q₅, q₆ and q₇ are each independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;
R₃ and R₄ are each independently, H, hydroxyl, halogen, C₁-C₆ alkyl, substituted C₁-C₆ alkyl, C₁-C₆ alkoxy or substituted C₁-C₆ alkoxy;
each substituted group comprises one or more optionally protected substituent groups independently selected from halogen, OJ₁, NJ₁J₂, SJ₁, N₃, OC(═X)J₁, OC(═X)NJ₁J₂, NJ₃C(═X)NJ₁J₂ and CN, wherein X is O, S or NJ₁ and each J₁, J₂ and J₃ is, independently, H or C₁-C₆ alkyl; and
wherein the oligomeric compound has a nucleobase sequence, and wherein at least a portion of the nucleobase sequence of the oligomeric compound is (a) complementary to a portion of a target non-coding RNA; (b) identical to a portion of a target non-coding RNA; or (c) complementary to a portion of a target pre-mRNA.

In certain such embodiments, the oligomeric compound comprises a 5′ wing region, a gap region, and a 3′ wing region. In certain embodiments, the compound comprises alternating motif. In certain embodiments, the compound is uniformly modified.

### DETAILED DESCRIPTION OF THE INVENTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

### I. DEFINITIONS

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 18th edition, 1990; and “Antisense Drug Technology, Principles, Strategies, and Applications” Edited by Stanley T. Crooke, CRC Press, Boca Raton, Fla.; and Sambrook et al., “Molecular Cloning, A laboratory Manual,” 2^{nd} Edition, Cold Spring Harbor Laboratory Press, 1989, which are hereby incorporated by reference for any purpose. Where permitted, all patents, applications, published applications and other publications and other data referred to throughout in the disclosure herein are incorporated by reference in their entirety.

Unless otherwise indicated, the following terms have the following meanings:

As used herein, “nucleoside” refers to a glycosylamine comprising a heterocyclic base moiety and a sugar moiety. Nucleosides include, but are not limited to, naturally occurring nucleosides, a basic nucleosides, modified nucleosides, and nucleosides having mimetic bases and/or sugar groups. Nucleosides may be modified with any of a variety of substituents.

As used herein, “sugar moiety” means a natural or modified sugar ring or sugar surrogate.

As used herein, “nucleotide” refers to a nucleoside comprising a phosphate linking group. As used herein, nucleosides include nucleotides.

As used herein, “nucleobase” refers to the heterocyclic base portion of a nucleoside. Nucleobases may be naturally occurring or may be modified. In certain embodiments, a nucleobase may comprise any atom or group of atoms capable of hydrogen bonding to a base of another nucleic acid.

As used herein, “modified nucleoside” refers to a nucleoside comprising at least one modification compared to naturally occurring RNA or DNA nucleosides. Such modification may be at the sugar moiety and/or at the nucleobases. Such modifications to the sugar moity of a modified nucleoside include substituted sugars, in which substituents of the pentofuranose ring are different from those of an unmodified RNA or DNA nucleoside and also includes sugar surrogates, in which the pentofuranose ring is replaced or internally modified.

sugar surrogates, in which the pentofuranose ring of an unmodified nucleoside

As used herein, “bicyclic nucleoside” or “BNA” refers to a nucleoside wherein the sugar moiety of the nucleoside comprises a bridge connecting two carbon atoms of the sugar ring, thereby forming a bicyclic sugar moiety.

As used herein, “4′-2′ bicyclic nucleoside” refers to a bicyclic nucleoside comprising a furanose ring comprising a bridge connecting two carbon atoms of the furanose ring connects the 2′ carbon atom and the 4′ carbon atom of the sugar ring.

As used herein, “2′-modified” or “2′-substituted” refers to a nucleoside comprising a sugar comprising a substituent at the 2′ position other than H or OH. 2′-modified nucleosides, include, but are not limited to, bicyclic nucleosides wherein the bridge connecting two carbon atoms of the sugar ring connects the 2′ carbon and another carbon of the sugar ring; and nucleosides with non-bridging 2′ substituents, such as allyl, amino, azido, thio, O-allyl, O—C₁-C₁₀ alkyl, —OCF₃, O—(CH₂)₂—O—CH₃, 2′-O(CH₂)₂SCH₃, O—(CH₂)₂—O—N(Rₘ)(Rₙ), or O—CH₂—C(═O)—N(Rₘ)(Rₙ), where each Rₘ and Rₙ is, independently, H or substituted or unsubstituted C₁-C₁₀ alkyl. 2′-modified nucleosides may further comprise other modifications, for example at other positions of the sugar and/or at the nucleobase.

As used herein, “2′-F” refers to a nucleoside comprising a sugar comprising a fluoro group at the 2′ position.

As used herein, “2′-OMe” or “2′-OCH₃” or “2′-O-methyl” each refers to a nucleoside comprising a sugar comprising an —OCH₃ group at the 2′ position of the sugar ring.

As used herein, “MOE” or “2′-MOE” or “2′-OCH₂CH₂OCH₃” or “2′-O-methoxyethyl” each refers to a nucleoside comprising a sugar comprising a —OCH₂CH₂OCH₃ group at the 2′ position of the sugar ring.

As used herein, “phosphorous moiety” refers to a group comprising a phosphate, phosphonate, alkylphosphonates, aminoalkyl phosphonate, phosphorothioate, phosphoramidite, alkylphosphonothioate, phosphorodithioate, thiophosphoramidate, phosphotriester or the like. Specifically, modified phosphorous moieties have the following structural formula:

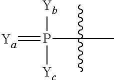

wherein Yₐ is O or S and each Y_{b} and Y_{c} is, independently, selected from OH, SH, alkyl, alkoxyl, substituted C₁-C₆ alkyl and substituted C₁-C₆ alkoxyl.

As used herein, “oligonucleotide” refers to a compound comprising a plurality of linked nucleosides. In certain embodiments, one or more of the plurality of nucleosides is modified. In certain embodiments, an oligonucleotide comprises one or more ribonucleosides (RNA) and/or deoxyribonucleosides (DNA).

As used herein “oligonucleoside” refers to an oligonucleotide in which none of the internucleoside linkages contains a phosphorus atom. As used herein, oligonucleotides include oligonucleosides.

As used herein, “modified oligonucleotide” refers to an oligonucleotide comprising at least one modified nucleoside and/or at least one modified internucleoside linkage.

As used herein “internucleoside linkage” refers to a covalent linkage between adjacent nucleosides.

As used herein “naturally occurring internucleoside linkage” refers to a 3′ to 5′ phosphodiester linkage.

As used herein, “modified internucleoside linkage” refers to any internucleoside linkage other than a naturally occurring internucleoside linkage.

As used herein, “oligomeric compound” refers to a polymeric structure comprising two or more sub-structures (“monomers”). In certain embodiments, an oligomeric compound is an oligonucleotide. In certain embodiments, an oligomeric compound is a single-stranded oligonucleotide. In certain embodiments, an oligomeric compound is a double-stranded duplex comprising two oligonucleotides. In certain embodiments, an oligomeric compound is a single-stranded or double-stranded oligonucleotide comprising one or more conjugate groups and/or terminal groups.

As used herein, “duplex” refers to two separate oligomeric compounds that are hybridized together.

As used herein, “terminal group” refers to one or more atom attached to either, or both, the 3′ end or the 5′ end of an oligonucleotide. In certain embodiments a terminal group is a conjugate group. In certain embodiments, a terminal group comprises one or more additional nucleosides.

As used herein, “conjugate” refers to an atom or group of atoms bound to an oligonucleotide or oligomeric compound. In general, conjugate groups modify one or more properties of the compound to which they are attached, including, but not limited to pharmakodynamic, pharmacokinetic, binding, absorption, cellular distribution, cellular uptake, charge and clearance. Conjugate groups are routinely used in the chemical arts and are linked directly or via an optional linking moiety or linking group to the parent compound such as an oligomeric compound. In certain embodiments, conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes. In certain embodiments, conjugates are terminal groups. In certain embodiments, conjugates are attached to a 3′ or 5′ terminal nucleoside or to an internal nucleosides of an oligonucleotide.

As used herein, “conjugate linking group” refers to any atom or group of atoms used to attach a conjugate to an oligonucleotide or oligomeric compound. Linking groups or bifunctional linking moieties such as those known in the art are amenable to the present invention.

As used herein, “protecting group,” as used herein, refers to a labile chemical moiety which is known in the art to protect reactive groups including without limitation, hydroxyl, amino and thiol groups, against undesired reactions during synthetic procedures. Protecting groups are typically used selectively and/or orthogonally to protect sites during reactions at other reactive sites and can then be removed to leave the unprotected group as is or available for further reactions. Protecting groups as known in the art are described generally in Greene and Wuts, Protective Groups in Organic Synthesis, 3rd edition, John Wiley & Sons, New York (1999).

As used herein, the term “orthogonally protected” refers to functional groups which are protected with different classes of protecting groups, wherein each class of protecting group can be removed in any order and in the presence of all other classes (see, Barmy, G. and Merrifield, R. B., J. Am. Chem. Soc., 1977, 99, 7363; idem, 1980, 102, 3084.) Orthogonal protection is widely used in for example automated oligonucleotide synthesis. A functional group is deblocked in the presence of one or more other protected functional groups which is not affected by the deblocking procedure. This deblocked functional group is reacted in some manner and at some point a further orthogonal protecting group is removed under a different set of reaction conditions. This allows for selective chemistry to arrive at a desired compound or oligomeric compound.

As used herein, “antisense compound” refers to an oligomeric compound, at least a portion of which is at least partially complementary to a target nucleic acid to which it hybridizes. In certain embodiments, an antisense compound modulates (increases or decreases) expression or amount of a target nucleic acid. In certain embodiments, an antisense compound alters splicing of a target pre-mRNA resulting in a different splice variant. In certain embodiments, an antisense compound modulates expression of one or more different target proteins. Antisense mechanisms contemplated herein include, but are not limited to an RNase H mechanism, RNAi mechanisms, splicing modulation, translational arrest, altering RNA processing, inhibiting microRNA function, or mimicking microRNA function.

As used herein, “expression” refers to the process by which a gene ultimately results in a protein. Expression includes, but is not limited to, transcription, splicing, post-transcriptional modification, and translation.

As used herein, “RNAi” refers to a mechanism by which certain antisense compounds effect expression or amount of a target nucleic acid. RNAi mechanisms involve the RISC pathway.

As used herein, “RNAi compound” refers to an oligomeric compound that acts through an RNAi mechanism to modulate a target nucleic acid and/or protein encoded by a target nucleic acid. RNAi compounds include, but are not limited to double-stranded short interfering RNA (siRNA), single-stranded RNA (ssRNA), and microRNA, including microRNA mimics.

As used herein, “antisense oligonucleotide” refers to an antisense compound that is an oligonucleotide.

As used herein, “antisense activity” refers to any detectable and/or measurable activity attributable to the hybridization of an antisense compound to its target nucleic acid. In certain embodiments, such activity may be an increase or decrease in an amount of a nucleic acid or protein. In certain embodiments, such activity may be a change in the ratio of splice variants of a nucleic acid or protein. Detection and/or measuring of antisense activity may be direct or indirect. For example, in certain embodiments, antisense activity is assessed by detecting and/or measuring the amount of target protein or the relative amounts of splice variants of a target protein. In certain embodiments, antisense activity is assessed by detecting and/or measuring the amount of target nucleic acids and/or cleaved target nucleic acids and/or alternatively spliced target nucleic acids. In certain embodiments, antisense activity is assessed by observing a phenotypic change in a cell or animal.

As used herein “detecting” or “measuring” in connection with an activity, response, or effect indicate that a test for detecting or measuring such activity, response, or effect is performed. Such detection and/or measuring may include values of zero. Thus, if a test for detection or measuring results in a finding of no activity (activity of zero), the step of detecting or measuring the activity has nevertheless been performed. For example, in certain embodiments, the present invention provides methods that comprise steps of detecting antisense activity, detecting toxicity, and/or measuring a marker of toxicity. Any such step may include values of zero.

As used herein, “target nucleic acid” refers to any nucleic acid molecule the expression, amount, or activity of which is capable of being modulated by an antisense compound. In certain embodiments, the target nucleic acid is DNA or RNA. In certain embodiments, the target RNA is mRNA, pre-mRNA, non-coding RNA, pri-microRNA, pre-microRNA, mature microRNA, promoter-directed RNA, or natural antisense transcripts. For example, the target nucleic acid can be a cellular gene (or mRNA transcribed from the gene) whose expression is associated with a particular disorder or disease state, or a nucleic acid molecule from an infectious agent. In certain embodiments, target nucleic acid is a viral or bacterial nucleic acid.

As used herein, “target mRNA” refers to a pre-selected RNA molecule that encodes a protein.

As used herein, “target pre-mRNA” refers to a pre-selected RNA transcript that has not been fully processed into mRNA. Notably, pre-RNA includes one or more intron.

As used herein, “target microRNA” refers to a pre-selected non-coding RNA molecule about 18-30 nucleobases in length that modulates expression of one or more proteins or to a precursor of such a non-coding molecule.

As used herein, “target pdRNA” refers to refers to a pre-selected RNA molecule that interacts with one or more promoter to modulate transcription.

As used herein, “pri-miRNA” or “pri-miR” refers to a non-coding RNA having a hairpin structure that is a substrate for the double-stranded RNA-specific ribonuclease Drosha.

As used herein, “miRNA precursor” refers to a transcript that originates from a genomic DNA and that comprises a non-coding, structured RNA comprising one or more miRNA sequences. For example, in certain embodiments a miRNA precursor is a pre-miRNA. In certain embodiments, a miRNA precursor is a pri-miRNA.

As used herein, “monocistronic transcript” refers to a miRNA precursor containing a single miRNA sequence.

As used herein, “polycistronic transcript” refers to a miRNA precursor containing two or more miRNA sequences.

As used herein, “microRNA” refers to a naturally occurring, small, non-coding RNA that represses gene expression at the level of translation. In certain embodiments, a microRNA represses gene expression by binding to a target site within a 3′ untranslated region of a target nucleic acid. In certain embodiments, a microRNA has a nucleobase sequence as set forth in miRBase, a database of published microRNA sequences found at http://microrna.sanger.ac.uk/sequences/. In certain embodiments, a microRNA has a nucleobase sequence as set forth in miRBase version 10.1 released December 2007, which is herein incorporated by reference in its entirety. In certain embodiments, a microRNA has a nucleobase sequence as set forth in miRBase version 12.0 released September 2008, which is herein incorporated by reference in its entirety.

As used herein, “microRNA mimic” refers to an oligomeric compound having a sequence that is at least partially identical to that of a microRNA. In certain embodiments, a microRNA mimic comprises the microRNA seed region of a microRNA. In certain embodiments, a microRNA mimic modulates translation of more than one target nucleic acids.

As used herein, “seed region” refers to a region at or near the 5′ end of an antisense compound having a nucleobase sequence that is import for target nucleic acid recognition by the antisense compound. In certain embodiments, a seed region comprises nucleobases 2-8 of an antisense compound. In certain embodiments, a seed region comprises nucleobases 2-7 of an antisense compound. In certain embodiments, a seed region comprises nucleobases 1-7 of an antisense compound. In certain embodiments, a seed region comprises nucleobases 1-6 of an antisense compound. In certain embodiments, a seed region comprises nucleobases 1-8 of an antisense compound.

As used herein, “microRNA seed region” refers to a seed region of a microRNA or microRNA mimic. In certain embodiments, a microRNA seed region comprises nucleobases 2-8 of a microRNA or microRNA mimic. In certain embodiments, a microRNA seed region comprises nucleobases 2-7 of a microRNA or microRNA mimic. In certain embodiments, a microRNA seed region comprises nucleobases 1-7 of a microRNA or microRNA mimic. In certain embodiments, a microRNA seed region comprises nucleobases 1-6 of a microRNA or microRNA mimic. In certain embodiments, a microRNA seed region comprises nucleobases 1-8 of a microRNA or microRNA mimic.

As used herein, “seed match segment” refers to a portion of a target nucleic acid having nucleobase complementarity to a seed region. In certain embodiments, a seed match segment has nucleobase complementarity to nucleobases 2-8 of an siRNA, ssRNA, natural microRNA or microRNA mimic. In certain embodiments, a seed match segment has nucleobase complementarity to nucleobases 2-7 of an siRNA, ssRNA, microRNA or microRNA mimic. In certain embodiments, a seed match segment has nucleobase complementarity to nucleobases 1-6 of an siRNA, ssRNA, microRNA or microRNA mimic. In certain embodiments, a seed match segment has nucleobase complementarity to nucleobases 1-7 of an siRNA, ssRNA, microRNA or microRNA mimic. In certain embodiments, a seed match segment has nucleobase complementarity to nucleobases 1-8 of an siRNA, ssRNA, microRNA or microRNA mimic.

As used herein, “seed match target nucleic acid” refers to a target nucleic acid comprising a seed match segment.

As used herein, “microRNA family” refers to a group of microRNAs that share a microRNA seed sequence. In certain embodiments, microRNA family members regulate a common set of target nucleic acids. In certain embodiments, the shared microRNA seed sequence is found at the same nucleobase positions in each member of a microRNA family. In certain embodiments, the shared microRNA seed sequence is not found at the same nucleobase positions in each member of a microRNA family. For example, a microRNA seed sequence found at nucleobases 1-7 of one member of a microRNA family may be found at nucleobases 2-8 of another member of a microRNA family.

As used herein, “target non-coding RNA” refers to a pre-selected RNA molecule that is not translated to generate a protein. Certain non-coding RNA are involved in regulation of expression.

As used herein, “target viral nucleic acid” refers to a pre-selected nucleic acid (RNA or DNA) associated with a virus. Such viral nucleic acid includes nucleic acids that constitute the viral genome, as well as transcripts (including reverse-transcripts and RNA transcribed from RNA) of those nucleic acids, whether or not produced by the host cellular machinery. In certain instances, viral nucleic acids also include host nucleic acids that are recruited by a virus upon viral infection.

As used herein, “targeting” or “targeted to” refers to the association of an antisense compound to a particular target nucleic acid molecule or a particular region of nucleotides within a target nucleic acid molecule. An antisense compound targets a target nucleic acid if it is sufficiently complementary to the target nucleic acid to allow hybridization under physiological conditions.

As used herein, “target site” refers to a region of a target nucleic acid that is bound by an antisense compound. In certain embodiments, a target site is at least partially within the 3′ untranslated region of an RNA molecule. In certain embodiments, a target site is at least partially within the 5′ untranslated region of an RNA molecule. In certain embodiments, a target site is at least partially within the coding region of an RNA molecule. In certain embodiments, a target site is at least partially within an exon of an RNA molecule. In certain embodiments, a target site is at least partially within an intron of an RNA molecule. In certain embodiments, a target site is at least partially within a microRNA target site of an RNA molecule. In certain embodiments, a target site is at least partially within a repeat region of an RNA molecule.

As used herein, “target protein” refers to a protein, the expression of which is modulated by an antisense compound. In certain embodiments, a target protein is encoded by a target nucleic acid.

In certain embodiments, expression of a target protein is otherwise influenced by a target nucleic acid.

As used herein, “nucleobase complementarity” refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair. Nucleobases comprising certain modifications may maintain the ability to pair with a counterpart nucleobase and thus, are still capable of nucleobase complementarity.

As used herein, “non-complementary nucleobase” refers to a pair of nucleobases that do not form hydrogen bonds with one another or otherwise support hybridization.

As used herein, “complementary” refers to the capacity of an oligomeric compound to hybridize to another oligomeric compound or nucleic acid through nucleobase complementarity. In certain embodiments, an antisense compound and its target are complementary to each other when a sufficient number of corresponding positions in each molecule are occupied by nucleobases that can bond with each other to allow stable association between the antisense compound and the target. One skilled in the art recognizes that the inclusion of mismatches is possible without eliminating the ability of the oligomeric compounds to remain in association. Therefore, described herein are antisense compounds that may comprise up to about 20% nucleotides that are mismatched (i.e., are not nucleobase complementary to the corresponding nucleotides of the target). Preferably the antisense compounds contain no more than about 15%, more preferably not more than about 10%, most preferably not more than 5% or no mismatches. The remaining nucleotides are nucleobase complementary or otherwise do not disrupt hybridization (e.g., universal bases). One of ordinary skill in the art would recognize the compounds provided herein are at least 80%, at least 85%, at least 90%, at least 95%, at least 96%, at least 97%, at least 98%, at least 99% or 100% complementary to a target nucleic acid.

As used herein, “hybridization” refers to the pairing of complementary oligomeric compounds (e.g., an antisense compound and its target nucleic acid or an antidote to its antisense compound). While not limited to a particular mechanism, the most common mechanism of pairing involves hydrogen bonding, which may be Watson-Crick, Hoogsteen or reversed Hoogsteen hydrogen bonding, between complementary nucleoside or nucleotide bases (nucleobases). For example, the natural base adenine is nucleobase complementary to the natural nucleobases thymidine and uracil which pair through the formation of hydrogen bonds. The natural base guanine is nucleobase complementary to the natural bases cytosine and 5-methyl cytosine. Hybridization can occur under varying circumstances.

As used herein, “specifically hybridizes” refers to the ability of an oligomeric compound to hybridize to one nucleic acid site with greater affinity than it hybridizes to another nucleic acid site. In certain embodiments, an antisense oligonucleotide specifically hybridizes to more than one target site.

As used herein, “overall identity” refers to the nucleobase identity of an oligomeric compound relative to a particular nucleic acid or portion thereof, over the length of the oligomeric compound.

As used herein, “modulation” refers to a perturbation of amount or quality of a function or activity when compared to the function or activity prior to modulation. For example, modulation includes the change, either an increase (stimulation or induction) or a decrease (inhibition or reduction) in gene expression. As a further example, modulation of expression can include perturbing splice site selection of pre-mRNA processing, resulting in a change in the amount of a particular splice-variant present compared to conditions that were not perturbed. As a further example, modulation includes perturbing translation of a protein.

As used herein, “motif” refers to a pattern of modifications in an oligomeric compound or a region thereof. Motifs may be defined by modifications at certain nucleosides and/or at certain linking groups of an oligomeric compound.

As used herein, “nucleoside motif” refers to a pattern of nucleoside modifications in an oligomeric compound or a region thereof. The linkages of such an oligomeric compound may be modified or unmodified. Unless otherwise indicated, motifs herein describing only nucleosides are intended to be nucleoside motifs. Thus, in such instances, the linkages are not limited.

As used herein, “linkage motif” refers to a pattern of linkage modifications in an oligomeric compound or region thereof. The nucleosides of such an oligomeric compound may be modified or unmodified. Unless otherwise indicated, motifs herein describing only linkages are intended to be linkage motifs. Thus, in such instances, the nucleosides are not limited.

As used herein, “different modifications” or “differently modified” refer to nucleosides or internucleoside linkages that have different nucleoside modifications or internucleoside linkages than one another, including absence of modifications. Thus, for example, a MOE nucleoside and an unmodified DNA nucleoside are “differently modified,” even though the DNA nucleoside is unmodified. Likewise, DNA and RNA are “differently modified,” even though both are naturally-occurring unmodified nucleosides. Nucleosides that are the same but for comprising different nucleobases are not differently modified, unless otherwise indicated. For example, a nucleoside comprising a 2′-OMe modified sugar and an adenine nucleobase and a nucleoside comprising a 2% OMe modified sugar and a thymine nucleobase are not differently modified.

As used herein, “the same modifications” refer to nucleosides and internucleoside linkages (including unmodified nucleosides and internucleoside linkages) that are the same as one another. Thus, for example, two unmodified DNA nucleoside have “the same modification,” even though the DNA nucleoside is unmodified.

As used herein, “type of modification” or nucleoside of a “type” refers to the modification of a nucleoside and includes modified and unmodified nucleosides. Accordingly, unless otherwise indicated, a “nucleoside having a modification of a first type” may be an unmodified nucleoside.

As used herein, “region” refers to a portion of an oligomeric compound wherein the nucleosides and internucleoside linkages within the region all comprise the same modifications; and the nucleosides and/or the internucleoside linkages of any neighboring portions include at least one different modification.

As used herein, “alternating motif” refers to an oligomeric compound or a portion thereof, having at lease four separate regions of modified nucleosides in a pattern (AB)ₙAₘ where A represents a region of nucleosides having a first type of modification; B represent a region of nucleosides having a different type of modification; n is 2-15; and m is 0 or 1. Thus, in certain embodiments, alternating motifs include 4, 5, 6, 7, 8, 9, 10, 11, 12, 13, 14, 15, 16, 17, 18, 19, or 20 or more alternating regions. In certain embodiments, each A region and each B region independently comprises 1-4 nucleosides.

As used herein, “fully modified” refers to an oligomeric compound or portion thereon wherein each nucleoside is a modified nucleoside. The modifications of the nucleosides of a fully modified oligomeric compound may all be the same or one or more may be different from one another.

As used herein, “uniform modified” or “uniformly modified” refer to oligomeric compounds or portions thereof that comprise the same modifications. The nucleosides of a region of uniformly modified nucleosides all comprise the same modification.

As used herein, “pharmaceutically acceptable salts” refers to salts of active compounds that retain the desired biological activity of the active compound and do not impart undesired toxicological effects thereto.

As used herein, “cap structure” or “terminal cap moiety” refers to chemical modifications incorporated at either terminus of an antisense compound.

As used herein, “mitigation” refers to a lessening of at least one activity or one indicator of the severity of a condition or disease. The severity of indicators may be determined by subjective or objective measures which are known to those skilled in the art. In certain embodiments, the condition may be a toxic effect of a therapeutic agent.

As used herein, “pharmaceutical agent” refers to a substance that provides a therapeutic effect when administered to a subject. In certain embodiments, a pharmaceutical agent provides a therapeutic benefit. In certain embodiments, a pharmaceutical agent provides a toxic effect.

As used herein, “therapeutic index” refers to the toxic dose of a drug for 50% of the population (TD₅₀) divided by the minimum effective dose for 50% of the population (ED₅₀). A high therapeutic index is preferable to a low one: this corresponds to a situation in which one would have to take a much higher amount of a drug to cause a toxic effect than the amount taken to cause a therapeutic benefit.

As used herein, “therapeutically effective amount” refers to an amount of a pharmaceutical agent that provides a therapeutic benefit to an animal.

As used herein, “administering” refers to providing a pharmaceutical agent to an animal, and includes, but is not limited to administering by a medical professional and self-administering.

As used herein, “co-administer” refers to administering more than one pharmaceutical agent to an animal. The more than one agent may be administered together or separately; at the same time or different times; through the same route of administration or through different routes of administration.

As used herein, “co-formulation” refers to a formulation comprising two or more pharmaceutically active agents. In certain embodiments, a co-formulation comprises two or more oligomeric compounds. In certain such embodiments, two or more oligomeric compound are oligomeric compounds of the present invention. In certain embodiments, one or more oligomeric compound present in a co-formulation is not a compound of the present invention. In certain embodiments, a co-formulation includes one or more non-oligomeric pharmaceutical agents.

As used herein, “route of administration” refers to the means by which a pharmaceutical agent is administered to an animal.

As used herein, “pharmaceutical composition” refers to a mixture of substances suitable for administering to an animal. For example, a pharmaceutical composition may comprise an antisense oligonucleotide and a sterile aqueous solution.

As used herein, “pharmaceutically acceptable carrier or diluent” refers to any substance suitable for use in administering to an animal. In certain embodiments, a pharmaceutically acceptable carrier or diluent is sterile saline. In certain embodiments, such sterile saline is pharmaceutical grade saline.

As used herein, “animal” refers to a human or a non-human animal, including, but not limited to, mice, rats, rabbits, dogs, cats, pigs, and non-human primates, including, but not limited to, monkeys and chimpanzees.

As used herein, “parenteral administration,” refers to administration through injection or infusion. Parenteral administration includes, but is not limited to, subcutaneous administration, intravenous administration, or intramuscular administration.

As used herein, “subcutaneous administration” refers to administration just below the skin. “Intravenous administration” refers to administration into a vein.

As used herein, “active pharmaceutical ingredient” refers to the substance in a pharmaceutical composition that provides a desired effect.

As used herein, “prodrug” refers to a therapeutic agent that is prepared in an inactive form that is converted to an active form (i.e., drug) within the body or cells thereof by the action of endogenous enzymes or other chemicals and/or conditions.

As used herein, “alkyl,” refers to a saturated straight or branched hydrocarbon radical containing up to twenty four carbon atoms. Examples of alkyl groups include, but are not limited to, methyl, ethyl, propyl, butyl, isopropyl, n-hexyl, octyl, decyl, dodecyl and the like. Alkyl groups typically include from 1 to about 24 carbon atoms, more typically from 1 to about 12 carbon atoms (C₁-C₁₂ alkyl) with from 1 to about 6 carbon atoms (C₁-C₆ alkyl) being more preferred. The term “lower alkyl” as used herein includes from 1 to about 6 carbon atoms (C₁-C₆ alkyl). Alkyl groups as used herein may optionally include one or more further substituent groups.

As used herein, “alkenyl,” refers to a straight or branched hydrocarbon chain radical containing up to twenty four carbon atoms and having at least one carbon-carbon double bond. Examples of alkenyl groups include, but are not limited to, ethenyl, propenyl, butenyl, 1-methyl-2-buten-1-yl, dienes such as 1,3-butadiene and the like. Alkenyl groups typically include from 2 to about 24 carbon atoms, more typically from 2 to about 12 carbon atoms with from 2 to about 6 carbon atoms being more preferred. Alkenyl groups as used herein may optionally include one or more further substituent groups.

As used herein, “alkynyl,” refers to a straight or branched hydrocarbon radical containing up to twenty four carbon atoms and having at least one carbon-carbon triple bond. Examples of alkynyl groups include, but are not limited to, ethynyl, 1-propynyl, 1-butynyl, and the like. Alkynyl groups typically include from 2 to about 24 carbon atoms, more typically from 2 to about 12 carbon atoms with from 2 to about 6 carbon atoms being more preferred. Alkynyl groups as used herein may optionally include one or more further substituent groups.

As used herein, “aminoalkyl” refers to an amino substituted alkyl radical. This term is meant to include C₁-C₁₂ alkyl groups having an amino substituent at any position and wherein the alkyl group attaches the aminoalkyl group to the parent molecule. The alkyl and/or amino portions of the aminoalkyl group can be further substituted with substituent groups.

As used herein, “aliphatic,” refers to a straight or branched hydrocarbon radical containing up to twenty four carbon atoms wherein the saturation between any two carbon atoms is a single, double or triple bond. An aliphatic group preferably contains from 1 to about 24 carbon atoms, more typically from 1 to about 12 carbon atoms with from 1 to about 6 carbon atoms being more preferred. The straight or branched chain of an aliphatic group may be interrupted with one or more heteroatoms that include nitrogen, oxygen, sulfur and phosphorus. Such aliphatic groups interrupted by heteroatoms include without limitation polyalkoxys, such as polyalkylene glycols, polyamines, and polyimines. Aliphatic groups as used herein may optionally include further substituent groups.

As used herein, “alicyclic” or “alicyclyl” refers to a cyclic ring system wherein the ring is aliphatic. The ring system can comprise one or more rings wherein at least one ring is aliphatic. Preferred alicyclics include rings having from about 5 to about 9 carbon atoms in the ring. Alicyclic as used herein may optionally include further substituent groups.

As used herein, “alkoxy,” refers to a radical formed between an alkyl group and an oxygen atom wherein the oxygen atom is used to attach the alkoxy group to a parent molecule. Examples of alkoxy groups include, but are not limited to, methoxy, ethoxy, propoxy, isopropoxy, n-butoxy, sec-butoxy, tert-butoxy, n-pentoxy, neopentoxy, n-hexoxy and the like. Alkoxy groups as used herein may optionally include further substituent groups.

As used herein, “halo” and “halogen,” refer to an atom selected from fluorine, chlorine, bromine and iodine.

As used herein, “aryl” and “aromatic,” refer to a mono- or polycyclic carbocyclic ring system radicals having one or more aromatic rings. Examples of aryl groups include, but are not limited to, phenyl, naphthyl, tetrahydronaphthyl, indanyl, idenyl and the like. Preferred aryl ring systems have from about 5 to about 20 carbon atoms in one or more rings. Aryl groups as used herein may optionally include further substituent groups.

As used herein, “aralkyl” and “arylalkyl,” refer to a radical formed between an alkyl group and an aryl group wherein the alkyl group is used to attach the aralkyl group to a parent molecule. Examples include, but are not limited to, benzyl, phenethyl and the like. Aralkyl groups as used herein may optionally include further substituent groups attached to the alkyl, the aryl or both groups that form the radical group.

As used herein, “heterocyclic radical” refers to a radical mono-, or poly-cyclic ring system that includes at least one heteroatom and is unsaturated, partially saturated or fully saturated, thereby including heteroaryl groups. Heterocyclic is also meant to include fused ring systems wherein one or more of the fused rings contain at least one heteroatom and the other rings can contain one or more heteroatoms or optionally contain no heteroatoms. A heterocyclic group typically includes at least one atom selected from sulfur, nitrogen or oxygen. Examples of heterocyclic groups include, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, tetrahydrofuryl and the like. Heterocyclic groups as used herein may optionally include further substitutent groups.

As used herein, “heteroaryl,” and “heteroaromatic,” refer to a radical comprising a mono- or poly-cyclic aromatic ring, ring system or fused ring system wherein at least one of the rings is aromatic and includes one or more heteroatom. Heteroaryl is also meant to include fused ring systems including systems where one or more of the fused rings contain no heteroatoms. Heteroaryl groups typically include one ring atom selected from sulfur, nitrogen or oxygen. Examples of heteroaryl groups include, but are not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like. Heteroaryl radicals can be attached to a parent molecule directly or through a linking moiety such as an aliphatic group or hetero atom. Heteroaryl groups as used herein may optionally include further substitutent groups.

As used herein, “heteroarylalkyl,” refers to a heteroaryl group as previously defined having an alky radical that can attach the heteroarylalkyl group to a parent molecule. Examples include, but are not limited to, pyridinylmethyl, pyrimidinylethyl, napthyridinylpropyl and the like. Heteroarylalkyl groups as used herein may optionally include further substitutent groups on one or both of the heteroaryl or alkyl portions.

As used herein, “mono or poly cyclic structure” refers to any ring systems that are single or polycyclic having rings that are fused or linked and is meant to be inclusive of single and mixed ring systems individually selected from aliphatic, alicyclic, aryl, heteroaryl, aralkyl, arylalkyl, heterocyclic, heteroaryl, heteroaromatic, heteroarylalkyl. Such mono and poly cyclic structures can contain rings that are uniform or have varying degrees of saturation including fully saturated, partially saturated or fully unsaturated. Each ring can comprise ring atoms selected from C, N, O and S to give rise to heterocyclic rings as well as rings comprising only C ring atoms which can be present in a mixed motif such as for example benzimidazole wherein one ring has only carbon ring atoms and the fused ring has two nitrogen atoms. The mono or poly cyclic structures can be further substituted with substituent groups such as for example phthalimide which has two ═O groups attached to one of the rings. In another aspect, mono or poly cyclic structures can be attached to a parent molecule directly through a ring atom, through a substituent group or a bifunctional linking moiety.

As used herein, “acyl,” refers to a radical formed by removal of a hydroxyl group from an organic acid and has the general formula —C(O)—X where X is typically aliphatic, alicyclic or aromatic. Examples include aliphatic carbonyls, aromatic carbonyls, aliphatic sulfonyls, aromatic sulfinyls, aliphatic sulfinyls, aromatic phosphates, aliphatic phosphates and the like. Acyl groups as used herein may optionally include further substitutent groups.

As used herein, “hydrocarbyl” refers to any group comprising C, O and H. Included are straight, branched and cyclic groups having any degree of saturation. Such hydrocarbyl groups can include one or more heteroatoms selected from N, O and S and can be further mono or poly substituted with one or more substituent groups.

As used herein, “substituent” and “substituent group,” include groups that are typically added to other groups or parent compounds to enhance desired properties or give desired effects. Substituent groups can be protected or unprotected and can be added to one available site or to many available sites in a parent compound. Substituent groups may also be further substituted with other substituent groups and may be attached directly or via a linking group such as an alkyl or hydro-carbyl group to a parent compound. Unless otherwise indicated, the term substituted or “optionally substituted” refers to the following substituents: halogen, hydroxyl, alkyl, alkenyl, alkynyl, acyl (—C—(O)Rₐₐ), carboxyl (—C(O)O—Rₐₐ), aliphatic groups, alicyclic groups, alkoxy, substituted oxo (—O—Rₐₐ), aryl, aralkyl, heterocyclic, heteroaryl, heteroarylalkyl, amino (—NR_{bb}R_{cc}), imino(═NR_{bb}), amido (—C(O)NR_{bb}R_{cc} or —N(R_{bb})C(O)Rₐₐ), azido (—N₃), nitro (—NO₂), cyano (—CN), carbamido (—OC(O)NR_{bb}R_{cc} or —N(R_{bb})C(O)ORₐₐ), ureido (—N(R_{bb})C(O)NR_{bb}R_{cc}), thioureido (—N(R_{bb})C—(S)NR_{bb}R_{cc}), guanidinyl (—N(R_{bb})C(═NR_{bb})NR_{bb}R_{cc}), amidinyl (—C(NR_{bb})NR_{bb}R_{cc} or —N(R_{bb})C(NR_{bb})Rₐₐ), thiol (—SR_{bb}), sulfinyl (—S(O)R_{bb}), sulfonyl (—S(O)₂R_{bb}), sulfonamidyl (—S(O)₂NR_{bb}R_{cc} or —N(R_{bb})S(O)₂R_{bb}) and conjugate groups. Wherein each Rₐₐ, R_{bb} and R_{cc} is, independently, H, an optionally linked chemical functional group or a further substituent group with a preferred list including without limitation H, alkyl, alkenyl, alkynyl, aliphatic, alkoxy, acyl, aryl, aralkyl, heteroaryl, alicyclic, heterocyclic and heteroarylalkyl.

As used herein, a zero (0) in a range indicating number of a particular unit means that the unit may be absent. For example, an oligomeric compound comprising 0-2 regions of a particular motif means that the oligomeric compound may comprise one or two such regions having the particular motif, or the oligomeric compound may not have any regions having the particular motif. In instances where an internal portion of a molecule is absent, the portions flanking the absent portion are bound directly to one another. Likewise, the term “none” as used herein, indicates that a certain feature is not present.

### III. CERTAIN OLIGOMERIC COMPOUNDS

In certain embodiments, the present invention provides oligomeric compounds. In certain embodiments, such oligomeric compounds are modified oligonucleotides. In certain embodiments, modified oligonucleotides of the present invention comprise modified nucleosides. In certain embodiments, modified oligonucleotides of the present invention comprise modified internucleoside linkages. In certain embodiments, modified oligonucleotides of the present invention comprise modified nucleosides and modified internucleoside linkages.

A. Certain Modified Nucleosides

In certain embodiments, modified oligonucleotides of the present invention comprise modified nucleosides comprising a modified sugar moiety. In certain embodiments, modified oligonucleotides of the present invention comprise modified nucleosides comprising a modified nucleobase. In certain embodiments, modified oligonucleotides of the present invention comprise modified nucleosides comprising a modified sugar moiety and a modified nucleobase.

1. Tetrahydropyran Nucleosides

In certain embodiments, the invention provides oligomeric compounds comprising one or more tetrahydropyran nucleoside analogs. In such embodiments, the furanose ring of a natural nucleoside is replaced with a substituted or unsubstituted tetrahydropyran ring. In certain embodiments, such tetrahydropyran nucleosides have the formula:

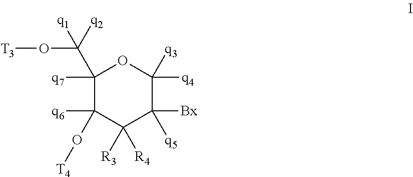

wherein independently for each of said tetrahydropyran nucleoside analogs of Formula I:

Bx is a heterocyclic base moiety;

T₃ and T₄ are each, independently, an internucleoside linking group linking the tetrahydropyran nucleoside analog to the oligomeric compound or one of T₃ and T₄ is an internucleoside linking group linking the tetrahydropyran nucleoside analog to the oligomeric compound and the other of T₃ and T₄ is H, a hydroxyl protecting group, a linked conjugate group or a 5′ or 3′-terminal group;

q₁, q₂, q₃, q₄, q₅, q₆ and q₇ are each independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;

R₃ and R₄ are each independently, H, hydroxyl, halogen, C₁-C₆ alkyl, substituted C₁-C₆ alkyl, C₁-C₆ alkoxy or substituted C₁-C₆ alkoxy;

each substituted group comprises one or more optionally protected substituent groups independently selected from halogen, OJ₁, NJ₁J₂, SJ₁, N₃, OC(═X)J₁, OC(═X)NJ₁J₂, NJ₃C(═X)NJ₁J₂ and CN, wherein X is O, S or NJ₁ and each J₁, J₂ and J₃ is, independently, H or C₁-C₆ alkyl.

2. Non-Tetrahydropyran Modified Nucleosides

i. Certain Modified Sugar Moieties

In certain embodiments, the present invention provides modified oligonucleotides comprising one or more nucleosides comprising a modified sugar moiety. In certain embodiments, a modified sugar moiety is a bicyclic sugar moiety. In certain embodiments a modified sugar moiety is a non-bicyclic modified sugar moiety.

Certain modified sugar moiety moieties are known and can be used to alter, typically increase, the affinity of the antisense compound for its target and/or increase nuclease resistance. A representative list of preferred modified sugar moieties includes but is not limited to bicyclic modified sugar moieties (BNA's), including methyleneoxy (4′-CH₂—O-2′) BNA, ethyleneoxy (4% (CH₂)₂—O-2′) BNA and methyl(methyleneoxy) (4′-C(CH₃)H—O-2′) BNA; substituted sugar moieties, especially 2′-substituted sugar moieties having a 2′-F, 2′-OCH₃ or a 2′-O(CH₂)₂—OCH₃ substituent group; and 4′-thio modified sugar moieties. Sugar moieties can also be replaced with sugar moiety mimetic groups among others. Methods for the preparations of modified sugar moieties are well known to those skilled in the art. Some representative patents and publications that teach the preparation of such modified sugar moieties include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; 6,531,584; 6,172,209; 6,271,358; and 6,600,032; and WO 2005/121371.

a. Certain Bicyclic Sugar Moieties

In certain embodiments, the present invention provides modified nucleosides comprising a bicyclic sugar moiety. Examples of bicyclic nucleosides include without limitation nucleosides comprising a bridge between the 4′ and the 2′ ribosyl ring atoms. In certain embodiments, oligomeric compounds provided herein include one or more bicyclic nucleosides wherein the bridge comprises one of the formulae: 4′-(CH2)-O-2′ (LNA); 4′-(CH2)-S-2; 4′-(CH2)₂—O-2′ (ENA); 4′-CH(CH3)-O-2′ and 4′-CH(CH2OCH3)-O-2′ (and analogs thereof see U.S. Pat. No. 7,399,845, issued on Jul. 15, 2008); 4′-C(CH3)(CH3)-O-2′ (and analogs thereof see published International Application WO/2009/006478, published Jan. 8, 2009); 4′-CH2-N(OCH3)-2′ (and analogs thereof see published International Application WO/2008/150729, published Dec. 11, 2008); 4′-CH2-O—N(CH3)-2′ (see published U.S. Patent Application US2004-0171570, published Sep. 2, 2004); 4′-CH2-N(R)—O-2′, wherein R is H, C1-C12 alkyl, or a protecting group (see U.S. Pat. No. 7,427,672, issued on Sep. 23, 2008); 4′-CH2-C(H)(CH3)-2′ (see Chattopadhyaya, et al., J. Org. Chem., 2009, 74, 118-134); and 4′-CH2-C(═CH2)-2′ (and analogs thereof see published International Application WO 2008/154401, published on Dec. 8, 2008). Each of the foregoing bicyclic nucleosides can be prepared having one or more stereochemical sugar configurations including for example α-L-ribofuranose and β-D-ribofuranose (see PCT international application PCT/DK98/00393, published on Mar. 25, 1999 as WO 99/14226). Certain such sugar moieties have been described. See, for example: Singh et al., Chem. Commun., 1998, 4, 455-456; Koshkin et al., Tetrahedron, 1998, 54, 3607-3630; Wahlestedt et al., Proc. Natl. Acad. Sci. U.S. A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 129(26) 8362-79 (Jul. 4, 2007); U.S. Pat. Nos. 7,053,207; 6,268,490; 6,770,748; 6,794,499; 7,034,133; and 6,525,191; Elayadi et al., Curr. Opinion Invens. Drugs, 2001, 2, 558-561; Braasch et al., Chem. Biol., 2001, 8 1-7; and Orum et al., Curr. Opinion Mol. Ther., 2001, 3, 239-243; and U.S. Pat. No. 6,670,461; International applications WO 2004/106356; WO 94/14226; WO 2005/021570; U.S. Patent Publication Nos. US2004-0171570; US2007-0287831; US2008-0039618; U.S. Pat. Nos. 7,399,845; U.S. patent Ser. Nos. 12/129,154; 60/989,574; 61/026,995; 61/026,998; 61/056,564; 61/086,231; 61/097,787; 61/099,844; PCT International Applications Nos. PCT/US2008/064591; PCT/US2008/066154; PCT/US2008/068922; and Published PCT International Applications WO 2007/134181; each of which is incorporated by reference in its entirety.

In certain embodiments, nucleosides comprising a bicyclic sugar moiety have increased affinity for a complementary nucleic acid. In certain embodiments, nucleosides comprising a bicyclic sugar moiety provide resistance to nuclease degradation of an oligonucleotide in which they are incorporated. For example, methyleneoxy (4′-CH₂—O-2′) BNA and other bicyclic sugar moiety analogs display duplex thermal stabilities with complementary DNA and RNA (Tm=+3 to +10° C.), stability towards 3′-exonucleolytic degradation and good solubility properties. Antisense oligonucleotides comprising BNAs have been described (Wahlestedt et al., Proc. Natl. Acad. Sci. U.S.A., 2000, 97, 5633-5638).

Certain bicyclic-sugar moiety containing nucleosides (or BNA nucleosides) comprise a bridge linking the 4′ carbon and the 2′ carbon of the sugar moiety. In certain embodiments, the bridging group is a methyleneoxy (4′-CH₂—O-2′). In certain embodiments, the bridging group is an ethyleneoxy (4′-CH₂CH₂—O-2′) (Singh et al., Chem. Commun., 1998, 4, 455-456: Morita et al., Bioorganic Medicinal Chemistry, 2003, 11, 2211-2226).

In certain embodiments, bicyclic sugar moieties of BNA nucleosides include, but are not limited to, compounds having at least one bridge between the 4′ and the 2′ position of the sugar moiety wherein such bridges independently comprises 1 or from 2 to 4 linked groups independently selected from —[C(Rₐ)(R_{b})]ₙ—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(R₁)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

In certain embodiments, the bridge of a bicyclic sugar moiety is, —[C(Rₐ)(R_{b})]ₙ—, —[C(Rₐ)(R_{b})]ₙ—O—, —C(RₐR_{b})—N(R₁)—O— or —C(RₐR_{b})—O—N(Rₐ)—. In certain embodiments, the bridge is 4′-CH₂-2′, 4′-(CH₂)₂-2′,4′-(CH₂)₃-2′,4′-CH₂—O-2′, 4′-(CH₂)₂—O-2′,4′-CH₂—O—N(Rₐ)-2′ and 4′-CH₂—N(Rₐ)—O-2′- wherein each Rₐ is, independently, H, a protecting group or C₁-C₁₂ alkyl. In certain embodiments, bicyclic nucleosides are further defined by isomeric configuration. For example, a nucleoside comprising a 4′-2′ methylenoxy bridge, may be in the α-L configuration or in the β-D configuration. Previously, alpha-L-methyleneoxy (4′-CH₂—O-2′) BNA's have been incorporated into antisense oligonucleotides that showed antisense activity (Frieden et al., Nucleic Acids Research, 2003, 21, 6365-6372).

In certain embodiments, bicyclic nucleosides include, but are not limited to, (A) α-L-Methyleneoxy (4′-CH₂—O-2′) BNA, (B) β-D-Methyleneoxy (4′-CH₂—O-2′) BNA, (C) Ethyleneoxy (4′-(CH₂)₂—O-2′) BNA, (D) Aminooxy (4′-CH₂—O—N(R)-2′) BNA, (E) Oxyamino (4′-CH₂—N(R)—O-2′) BNA, and (F) Methyl(methyleneoxy) (4′-C(CH₃)H—O-2′) BNA, as depicted below.

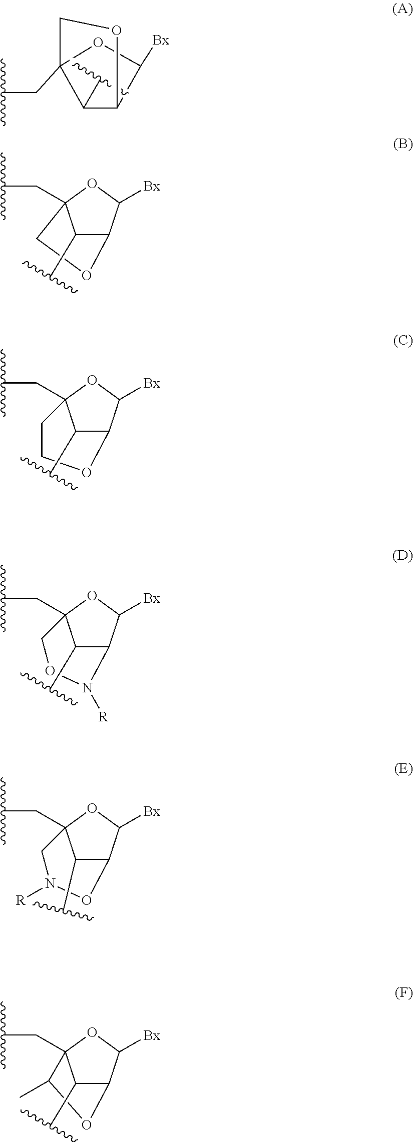

wherein Bx is the base moiety. In certain embodiments, bicyclic nucleosides include, but are not limited to, the structures below:

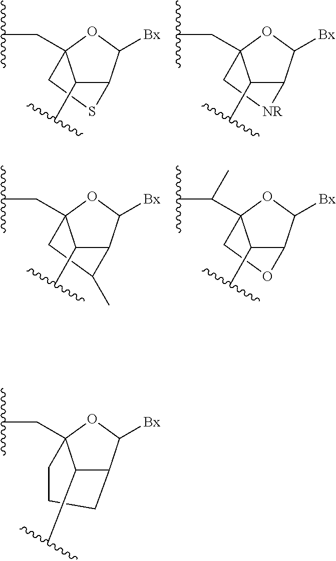

wherein Bx is the base moiety.

In certain embodiments, bicyclic nucleoside having the formula:

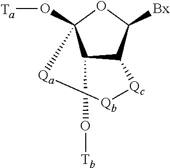

wherein

Bx is a heterocyclic base moiety;

-Qₐ-Q_{b}-Q_{c}- is —CH₂—N(R_{c})—CH₂—, —C(═O)—N(R_{c})—CH₂—, —CH₂—O—N(R_{c})— or N(R_{c})—O—CH₂—;

R_{c} is C₁-C₁₂ alkyl or an amino protecting group; and

Tₐ and T_{b} are each, independently, hydroxyl, a protected hydroxyl, a conjugate group, an activated phosphorus moiety or a covalent attachment to a support medium.

In certain embodiments, bicyclic nucleoside having the formula:

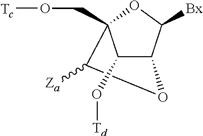

wherein:

Bx is a heterocyclic base moiety;

T_{c} is H or a hydroxyl protecting group;

T_{d} is H, a hydroxyl protecting group or a reactive phosphorus group;

Zₐ is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, or substituted amide.

In one embodiment, each of the substituted groups, is, independently, mono or poly substituted with optionally protected substituent groups independently selected from halogen, oxo, hydroxyl, OJ_{c}, NJ_{c}J_{d}, SJ_{c}, N₃, OC(═X)J_{c}, OC(═X)NJ_{c}J_{d}, NJₑC(═X)NJ_{c}J_{d} and CN, wherein each J_{c}, J_{d} and Jₑ is, independently, H or C₁-C₆ alkyl, and X is O, S or NJ_{c}.

In one embodiment, each of the substituted groups, is, independently, mono or poly substituted with substituent groups independently selected from halogen, oxo, hydroxyl, OJ_{c}, NJ_{c}J_{d}, SJ_{c}, N₃, OC(═X)J_{c}, and NJₑC(═X)NJ_{c}J_{d}, wherein each J_{c}, J_{d} and Jₑ is, independently, H, C₁-C₆ alkyl, or substituted C₁-C₆ alkyl and X is O or NJ_{c}.

In one embodiment, the Zₐ group is C₁-C₆ alkyl substituted with one or more X^{x}, wherein each X^{x} is independently OJ_{c}, NJ_{c}J_{d}, SJ_{c}, N₃, OC(═X)J_{c}, OC(═X)NJ_{c}J_{d}, NJₑC(═X)NJ_{c}J_{d} or CN; wherein each J_{c}, J_{d} and Jₑ is, independently, H or C₁-C₆ alkyl, and X is O, S or NJ_{c}. In another embodiment, the Zₐ group is C₁-C₆ alkyl substituted with one or more X^{x}, wherein each X^{x} is independently halo (e.g., fluoro), hydroxyl, alkoxy (e.g., CH₃O—), substituted alkoxy or azido.

In certain embodiments, bicyclic nucleoside having the formula:

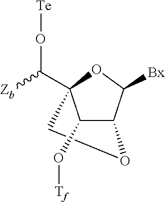

wherein:

Bx is a heterocyclic base moiety; one of Tₑ and T_{f} is H or a hydroxyl protecting group and the other of Tₑ and T_{f} is H, a hydroxyl protecting group or a reactive posphorus group;

Z_{b} is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl or substituted acyl (C(═O)—);

wherein each substituted group is mono or poly substituted with substituent groups independently selected from halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, OJ₁, SJ₁, NJ_{f}J_{g}, N₃, COOJ_{f}, CN, O—C(═O)NJ_{f}J_{g}, N(H)C(═NH)NR_{d}Rₑ or N(H)C(═X)N(H)J_{g} wherein X is O or S; and

each J_{f} and J_{g} is, independently, H, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, C₁-C₆ aminoalkyl, substituted C₁-C₆ aminoalkyl or a protecting group.

In certain embodiments, bicyclic nucleoside having the formula:

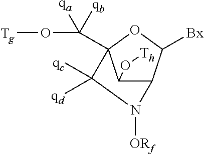

wherein:

Bx is a heterocyclic base moiety;

one of T_{g} and Tₕ is H or a hydroxyl protecting group and the other of T_{g} and Tₕ is H, a hydroxyl protecting group or a reactive phosphorus group;

R_{f} is C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;

qₐ and qₕ are each independently, H, halogen, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl, C₁-C₆ alkoxyl, substituted C₁-C₆ alkoxyl, acyl, substituted acyl, C₁-C₆ aminoalkyl or substituted C₁-C₆ aminoalkyl;

q_{c} and q_{d} are each independently, H, halogen, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl, C₁-C₆ alkoxyl, substituted C₁-C₆ alkoxyl, acyl, substituted acyl, C₁-C₆ aminoalkyl or substituted C₁-C₆ aminoalkyl;

wherein each substituted group is, independently, mono or poly substituted with substituent groups independently selected from halogen, OJₕ, SJₕ, NJₕJᵢ, N₃, COOJₕ, CN, O—C(═O)NJₕJᵢ, N(H)C(═NH)NJₕJᵢ or N(H)C(═X)N(H)Jᵢ wherein X is O or S; and

each Jₕ and Jᵢ is, independently, H, C₁-C₆ alkyl, C₂-C₆ alkenyl, C₂-C₆ alkynyl, C₁-C₆ aminoalkyl or a protecting group.

In certain embodiments, bicyclic nucleoside having the formula:

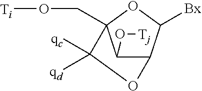

wherein:

Bx is a heterocyclic base moiety;

one of Tᵢ and Tᵢ is H or a hydroxyl protecting group and the other of Tᵢ and Tᵢ is H, a hydroxyl protecting group or a reactive phosphorus group;

qₑ and q_{f} are each, independently, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJ_{T}, SJ_{R}, SOJ_{A}, SO₂Jᵢ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ;

or qₑ and q_{f} together are ═C(q_{g})(qₕ);

q_{g} and qₕ are each, independently, H, halogen, C₁-C₁₂ alkyl or substituted C₁-C₁₂ alkyl;

each substituted group is, independently, mono or poly substituted with substituent groups independently selected from halogen, C₁-C₆ alkyl, C₂-C₆ alkenyl, C₂-C₆ alkynyl, OJⱼ, SJⱼ, NJⱼJₖ, N₃, CN, C(═O)OJⱼ, C(═O)NJⱼJₖ, O—C(═O)NJⱼJₖ, N(H)C(═O)NJⱼJₖ or N(H)C(═S)NJⱼJₖ; and

each Jⱼ and Jₖ is, independently, H, C₁-C₆ alkyl, C₂-C₆ alkenyl, C₂-C₆ alkynyl, C₁-C₆ aminoalkyl or a protecting group.

The synthesis and preparation of the methyleneoxy (4′-CH₂—O-2′) BNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O-2′) BNA, methyleneoxy (4′-CH₂—O-2′) BNA and 2′-thio-BNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-BNA, a novel comformationally restricted high-affinity oligonucleotide analog has been described in the art (Singh et al., J. Org. Chem., 1998, 63, 10035-10039). In addition, 2′-Amino- and 2′-methylamino-BNA's have been prepared and the thermal stability of their duplexes with complementary RNA and DNA strands has been previously reported.

b. Certain Non-Bicyclic Modified Sugar Moieties

In certain embodiments, the present invention provides modified nucleosides comprising modified sugar moieties that are not bicyclic sugar moieties. Certain such modified nucleosides are known. In certain embodiments, the sugar ring of a nucleoside may be modified at any position. Examples of sugar modifications useful in this invention include, but are not limited to compounds comprising a sugar substituent group selected from: OH, F, O-alkyl, S-alkyl, N-alkyl, or O-alkyl-O-alkyl, wherein the alkyl, alkenyl and alkynyl may be substituted or unsubstituted C₁ to C₁₀ alkyl or C₂ to C₁₀ alkenyl and alkynyl. In certain such embodiments, such substituents are at the 2′ position of the sugar.

In certain embodiments, modified nucleosides comprise a substituent at the 2′ position of the sugar. In certain embodiments, such substituents are selected from among: a halide, including, but not limited to F, allyl, amino, azido, thio, O-allyl, O—C₁-C₁₀ alkyl, —OCF₃, O—(CH₂)₂—O—CH₃, 2′-O(CH₂)₂SCH₃, O—(CH₂)₂—O—N(Rₘ)(Rₙ), or O—CH₂—C(═O)—N(Rₘ)(Rₙ), where each Rₘ and Rₙ is, independently, H or substituted or unsubstituted C₁-C₁₀ alkyl.

In certain embodiments, modified nucleosides suitable for use in the present invention are: 2-methoxyethoxy, 2′-O-methyl (2′-O—CH₃), 2′-fluoro (2′-F).

In certain embodiments, modified nucleosides having a substituent group at the 2′-position selected from: O[(CH₂)ₙO]ₘCH₃, O(CH₂)ₙNH₂, O(CH₂)ₙCH₃, O(CH₂)ₙONH₂, OCH₂C(═O)N(H)CH₃, and O(CH₂)ₙON[(CH₂)ₙCH₃]₂, where n and m are from 1 to about 10. Other 2′-sugar substituent groups include: C₁ to C₁₀ alkyl, substituted alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving pharmacokinetic properties, or a group for improving the pharmacodynamic properties of an oligomeric compound, and other substituents having similar properties.

In certain embodiments, modified nucleosides comprise a 2′-MOE side chain (Baker et al., J. Biol. Chem., 1997, 272, 11944-12000). Such 2′-MOE substitution have been described as having improved binding affinity compared to unmodified nucleosides and to other modified nucleosides, such as 2′-O-methyl, O-propyl, and O-aminopropyl. Oligonucleotides having the 2′-MOE substituent also have been shown to be antisense inhibitors of gene expression with promising features for in vivo use (Martin, P., Hely. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926).

In certain embodiments, 2′-Sugar substituent groups are in either the arabino (up) position or ribo (down) position. In certain such embodiments, a 2′-arabino modification is 2′-F arabino (FANA). Similar modifications can also be made at other positions on the sugar, particularly the 3′ position of the sugar on a 3′ terminal nucleoside or in 2′-5′ linked oligonucleotides and the 5′ position of 5′ terminal nucleotide.

In certain embodiments, nucleosides suitable for use in the present invention have sugar surrogates such as cyclobutyl in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, each of which is herein incorporated by reference in its entirety.

In certain embodiments, the present invention provides nucleosides comprising a modification at the 2′-position of the sugar. In certain embodiments, the invention provides nucleosides comprising a modification at the 5′-position of the sugar. In certain embodiments, the invention provides nucleosides comprising modifications at the 2′-position and the 5′-position of the sugar. In certain embodiments, modified nucleosides may be useful for incorporation into oligonucleotides. In certain embodiment, modified nucleosides are incorporated into oligonucleosides at the 5′-end of the oligonucleotide.

2. Certain Modified Nucleobases

In certain embodiments, nucleosides of the present invention comprise unmodified nucleobases. In certain embodiments, nucleosides of the present invention comprise modified nucleobases.

In certain embodiments, nucleobase modifications can impart nuclease stability, binding affinity or some other beneficial biological property to the oligomeric compounds. As used herein, “unmodified” or “natural” nucleobases include the purine bases adenine (A) and guanine (G), and the pyrimidine bases thymine (T), cytosine (C) and uracil (U). Modified nucleobases also referred to herein as heterocyclic base moieties include other synthetic and natural nucleobases, many examples of which such as 5-methylcytosine (5-me-C), 5-hydroxymethyl cytosine, 7-deazaguanine and 7-deazaadenine among others.

Heterocyclic base moieties can also include those in which the purine or pyrimidine base is replaced with other heterocycles, for example 7-deaza-adenine, 7-deazaguanosine, 2-aminopyridine and 2-pyridone. Certain modified nucleobases are disclosed in, for example, Swayze, E. E. and Bhat, B., The medicinal Chemistry of Oligonucleotides in ANTISENSE DRUG TECHNOLOGY, Chapter 6, pages 143-182 (Crooke, S. T., ed., 2008); U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2 aminopropyladenine, 5-propynyluracil and 5-propynylcytosine.

In certain embodiments, nucleobases comprise polycyclic heterocyclic compounds in place of one or more heterocyclic base moieties of a nucleobase. A number of tricyclic heterocyclic compounds have been previously reported. These compounds are routinely used in antisense applications to increase the binding properties of the modified strand to a target strand. The most studied modifications are targeted to guanosines hence they have been termed G-clamps or cytidine analogs.

Representative cytosine analogs that make 3 hydrogen bonds with a guanosine in a second strand include 1,3-diazaphenoxazine-2-one (Kurchavov, et al., Nucleosides and Nucleotides, 1997, 16, 1837-1846), 1,3-diazaphenothiazine-2-one (Lin, K.-Y.; Jones, R. J.; Matteucci, M. J. Am. Chem. Soc. 1995, 117, 3873-3874) and 6,7,8,9-tetrafluoro-1,3-diazaphenoxazine-2-one (Wang, J.; Lin, K.-Y., Matteucci, M. Tetrahedron Lett. 1998, 39, 8385-8388). When incorporated into oligonucleotides, these base modifications have been shown to hybridize with complementary guanine and the latter was also shown to hybridize with adenine and to enhance helical thermal stability by extended stacking interactions (also see U.S. Patent Application Publication 20030207804 and U.S. Patent Application Publication 20030175906, both of which are incorporated herein by reference in their entirety).

Helix-stabilizing properties have been observed when a cytosine analog/substitute has an aminoethoxy moiety attached to the rigid 1,3-diazaphenoxazine-2-one scaffold (Lin, K.-Y.; Matteucci, M. J. Am. Chem. Soc. 1998, 120, 8531-8532). Binding studies demonstrated that a single incorporation could enhance the binding affinity of a model oligonucleotide to its complementary target DNA or RNA with a ΔTₘ of up to 18° relative to 5-methyl cytosine (dC5^{me}), which is the highest known affinity enhancement for a single modification. On the other hand, the gain in helical stability does not compromise the specificity of the oligonucleotides. The T. data indicate an even greater discrimination between the perfect match and mismatched sequences compared to dC5^{me}. It was suggested that the tethered amino group serves as an additional hydrogen bond donor to interact with the Hoogsteen face, namely the O6, of a complementary guanine thereby forming 4 hydrogen bonds. This means that the increased affinity of G-clamp is mediated by the combination of extended base stacking and additional specific hydrogen bonding.

Tricyclic heterocyclic compounds and methods of using them that are amenable to the present invention are disclosed in U.S. Pat. No. 6,028,183, and U.S. Pat. No. 6,007,992, the contents of both are incorporated herein in their entirety.

The enhanced binding affinity of the phenoxazine derivatives together with their sequence specificity makes them valuable nucleobase analogs for the development of more potent antisense-based drugs. The activity enhancement was even more pronounced in case of G-clamp, as a single substitution was shown to significantly improve the in vitro potency of a 20mer 2′-deoxyphosphorothioate oligonucleotides (Flanagan, W. M.; Wolf, J. J.; Olson, P.; Grant, D.; Lin, K.-Y.; Wagner, R. W.; Matteucci, M. Proc. Natl. Acad. Sci. USA, 1999, 96, 3513-3518).

Modified polycyclic heterocyclic compounds useful as heterocyclic bases are disclosed in but not limited to, the above noted U.S. Pat. No. 3,687,808, as well as U.S. Pat. Nos. 4,845,205; 5,130,302; 5,134,066; 5,175,273; 5,367,066; 5,432,272; 5,434,257; 5,457,187; 5,459,255; 5,484,908; 5,502,177; 5,525,711; 5,552,540; 5,587,469; 5,594,121, 5,596,091; 5,614,617; 5,645,985; 5,646,269; 5,750,692; 5,830,653; 5,763,588; 6,005,096; and 5,681,941, and U.S. Patent Application Publication 20030158403, each of which is incorporated herein by reference in its entirety.

3. Certain Internucleoside Linkages

In such embodiments, nucleosides may be linked together using any internucleoside linkage. The two main classes of internucleoside linking groups are defined by the presence or absence of a phosphorus atom. Representative phosphorus containing internucleoside linkages include, but are not limited to, phosphodiesters (P═O), phosphotriesters, methylphosphonates, phosphoramidate, and phosphorothioates (P═S). Representative non-phosphorus containing internucleoside linking groups include, but are not limited to, methylenemethylimino (—CH₂—N(CH₃)—O—CH₂—), thiodiester (—O—C(O)—S—), thionocarbamate (—O—C(O)(NH)—S—); siloxane Si(H)₂—O—); and N,N′-dimethylhydrazine (—CH₂—N(CH₃)—N(CH₃)—). Oligonucleotides having non-phosphorus internucleoside linking groups may be referred to as oligonucleosides. Modified linkages, compared to natural phosphodiester linkages, can be used to alter, typically increase, nuclease resistance of the oligomeric compound. In certain embodiments, internucleoside linkages having a chiral atom can be prepared a racemic mixtures, as separate enantomers. Representative chiral linkages include, but are not limited to, alkylphosphonates and phosphorothioates. Methods of preparation of phosphorous-containing and non-phosphorous-containing internucleoside linkages are well known to those skilled in the art.

The oligonucleotides described herein contain one or more asymmetric centers and thus give rise to enantomers, diastereomers, and other stereoisomeric configurations that may be defined, in terms of absolute stereochemistry, as (R) or (S), α or β such as for sugar anomers, or as (D) or (L) such as for amino acids et al. Included in the antisense compounds provided herein are all such possible isomers, as well as their racemic and optically pure forms.

B. Lengths of Oligomeric Compounds

In certain embodiments, the invention provides oligomeric compounds comprising oligonucleotides. In certain embodiments, the present invention provides oligomeric compounds including oligonucleotides of any of a variety of ranges of lengths. In certain embodiments, the invention provides oligomeric compounds comprising oligonucleotides consisting of X to Y linked nucleosides, where X represents the fewest number of nucleosides in the range and Y represents the largest number of nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X<Y. For example, in certain embodiments, the invention provides oligomeric compounds which comprise oligonucleotides consisting of 8 to 9, 8 to 10, 8 to 11, 8 to 12, 8 to 13, 8 to 14, 8 to 15, 8 to 16, 8 to 17, 8 to 18, 8 to 19, 8 to 20, 8 to 21, 8 to 22, 8 to 23, 8 to 24, 8 to 25, 8 to 26, 8 to 27, 8 to 28, 8 to 29, 8 to 30, 9 to 10, 9 to 11, 9 to 12, 9 to 13, 9 to 14, 9 to 15, 9 to 16, 9 to 17, 9 to 18, 9 to 19, 9 to 20, 9 to 21, 9 to 22, 9 to 23, 9 to 24, 9 to 25, 9 to 26, 9 to 27, 9 to 28, 9 to 29, 9 to 30, 10 to 11, 10 to 12, 10 to 13, 10 to 14, 10 to 15, 10 to 16, 10 to 17, 10 to 18, 10 to 19, 10 to 20, 10 to 21, 10 to 22, 10 to 23, 10 to 24, 10 to 25, 10 to 26, 10 to 27, 10 to 28, 10 to 29, 10 to 30, 11 to 12, 11 to 13, 11 to 14, 11 to 15, 11 to 16, 11 to 17, 11 to 18, 11 to 19, 11 to 20, 11 to 21, 11 to 22, 11 to 23, 11 to 24, 11 to 25, 11 to 26, 11 to 27, 11 to 28, 11 to 29, 11 to 30, 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides. In embodiments where the number of nucleosides of an oligomeric compound or oligonucleotide is limited, whether to a range or to a specific number, the oligomeric compound or oligonucleotide may, nonetheless further comprise additional other substituents. For example, an oligonucleotide consisting of 8-30 nucleosides excludes oligonucleotides having 31 nucleosides, but, unless otherwise indicated, such an oligonucleotide may further comprise, for example one or more conjugates, terminal groups, or other substituents. In certain embodiments, terminal groups include, but are not limited to, terminal group nucleosides. In such embodiments, the terminal group nucleosides are differently modified than the terminal nucleoside of the oligonucleotide, thus distinguishing such terminal group nucleosides from the nucleosides of the oligonucleotide.

Motifs of Oligomeric Compounds

In certain embodiments, oligomeric compounds can have chemically modified subunits arranged in specific orientations along their length. A “chemical motif” is defined as the arrangement of chemical modifications throughout an oligomeric compound

In certain embodiments, oligomeric compounds of the invention are uniformly modified. As used herein, in a “uniformly modified” oligomeric compound a chemical modification of a sugar, base, internucleoside linkage, or combination thereof, is applied to each subunit of the oligomeric compound. In one embodiment, each sugar moiety of a uniformly modified oligomeric compound is modified. In other embodiments, each internucleoside linkage of a uniformly modified oligomeric compound is modified. In further embodiments, each sugar and each internucleoside linkage of uniformly modified oligomeric compounds bears a modification. Examples of uniformly modified oligomeric compounds include, but are not limited to, uniform 2′-MOE sugar moieties; uniform 2′-MOE and uniform phosphorothioate backbone; uniform 2′-OMe; uniform 2′-OMe and uniform phosphorothioate backbone; uniform 2′-F; uniform 2′-F and uniform phosphorothioate backbone; uniform phosphorothioate backbone; uniform deoxynucleotides; uniform ribonucleotides; uniform phosphorothioate backbone; and combinations thereof.

As used herein the term “positionally modified motif” is meant to include a sequence of uniformly sugar modified nucleosides wherein the sequence is interrupted by two or more regions comprising from 1 to about 8 sugar modified nucleosides wherein internal regions are generally from 1 to about 6 or from 1 to about 4. The positionally modified motif includes internal regions of sugar modified nucleoside and can also include one or both termini. Each particular sugar modification within a region of sugar modified nucleosides essentially uniform. The nucleotides of regions are distinguished by differing sugar modifications. Positionally modified motifs are not determined by the nucleobase sequence or the location or types of internucleoside linkages. The term positionally modified oligomeric compound includes many different specific substitution patterns. A number of these substitution patterns have been prepared and tested in compositions. In one embodiment the positionally modified oligomeric compounds may comprise phosphodiester internucleotide linkages, phosphorothioate internucleotide linkages, or a combination of phosphodiester and phosphorothioate internucleotide linkages.

In some embodiments, positionally modified oligomeric compounds include oligomeric compounds having clusters of a first modification interspersed with a second modification, as follows 5′-MMmmMmMMMmmmmMMMmmmmm-3′; and 5% MMmMMmMMmMMmMMmMMmMMmMM-3′; wherein “M” represent the first modification, and “m” represents the second modification. In one embodiment, “M” is 2′-MOE and “m” is a tetrahydropyran nucleoside. In other embodiments, “M” is 2′-F and “m” is tetrahydropyran. In other embodiments, “M” is tetrahydropyran nucleoside and “m” is 2′-MOE.

In certain embodiments, oligomeric compounds are gapmers. The types of sugar moieties that are used to differentiate the regions of a gapmer oligomeric compound include β-D-ribonucleosides, β-D-deoxyribonucleosides, or 2′-modified nucleosides disclosed herein, including, without limitation, 2′-MOE, 2′-fluoro, 2′-O—CH3, and bicyclic sugar modified nucleosides. In one embodiment, each region is uniformly modified. In another embodiment, the nucleosides of the internal region uniform sugar moieties that are different than the sugar moieties in an external region. In one non-limiting example, the gap is uniformly comprised of a first 2′-modified nucleoside and each of the wings is uniformly comprised of a second 2′-modified nucleoside.

Gapmer oligomeric compounds are further defined as being either “symmetric” or “asymmetric”. A gapmer having the same uniform sugar modification in each of the wings is termed a “symmetric gapmer oligomeric compound.” A gapmer having different uniform modifications in each wing is termed an “asymmetric gapmer oligomeric compound.” In one embodiment, gapmer oligomeric compounds such as these can have, for example, both wings comprising 2′-MOE modified nucleosides (symmetric gapmer) and a gap comprising β-D-ribonucleosides or β-D-deoxyribonucleosides. In another embodiment, a symmetric gapmer can have both wings comprising 2′-MOE modified nucleosides and a gap comprising 2′-modified nucleosides other than 2′-MOE modified nucleosides. Asymmetric gapmer oligomeric compounds, for example, can have one wing comprising 2′-OCH3 modified nucleosides and the other wing comprising 2′-MOE modified nucleosides with the internal region (gap) comprising 13-D-ribonucleosides, β-D-deoxyribonucleosides or 2′-modified nucleosides that are other than 2′-MOE or 2′-OCH3 modified nucleosides. These gapmer oligomeric compounds may comprise phosphodiester internucleotide linkages, phosphorothioate internucleotide linkages, or a combination of phosphodiester and phosphorothioate internucleotide linkages.

In some embodiments, each wing of a gapmer oligomeric compounds comprises the same number of subunits. In other embodiments, one wing of a gapmer oligomeric compound comprises a different number of subunits than the other wing of a gapmer oligomeric compound. In one embodiment, the wings of gapmer oligomeric compounds have, independently, from 1 to about 3 nucleosides. Suitable wings comprise from 2 to about 3 nucleosides. In one embodiment, the wings can comprise 2 nucleosides. In another embodiment, the 5′-wing can comprise 1 or 2 nucleosides and the 3′-wing can comprise 2 or 3 nucleosides. The present invention therefore includes gapped oligomeric compounds wherein each wing independently comprises 1, 2 or 3 sugar modified nucleosides. In one embodiment, the internal or gap region comprises from 15 to 23 nucleosides, which is understood to include 15, 16, 17, 18, 19, 20, 21, 22 and 23 nucleotides. In a further embodiment, the internal or gap region is understood to comprise from 17 to 21 nucleosides, which is understood to include 17, 18, 19, 20, or 21 nucleosides. In another embodiment, the internal or gap region is understood to comprise from 18 to 20 nucleosides, which is understood to include 18, 19 or 20 nucleosides. In one preferred embodiment, the gap region comprises 19 nucleosides. In one embodiment, the oligomeric compound is a gapmer oligonucleotides with full length complementarity to its target miRNA. In a further embodiment, the wings are 2′-MOE modified nucleosides and the gap comprises 2′-fluoro modified nucleosides. In one embodiment one wing is 2 nucleosides in length and the other wing is 3 nucleosides in length. In an additional embodiment, the wings are each 2 nucleosides in length and the gap region is 19 nucleotides in length.

Examples of oligomeric compounds include, but are not limited to, a 23 nucleobase oligomeric compound having a central region comprised of a first modification and wing regions comprised of a second modification (5′MMmmmmmmmmmmmmmmmmmmmMM3′); a 22 nucleobase compound having a central region comprised of a first modification and wing regions comprised of a second modification (5′MMmmmmmmmmmmmmmmmmmmMM3′); and a 21 nucleobase compound having a central region comprised of a first modification and wing regions comprised of a second modification (5′MMmmmmmmmmmmmmmmmmmMM3′); wherein “M” represents the first modification and “m” represents the second modification. In one non-limiting example, “M” may be 2′-O-methoxyethyl and “m” may be 2′-fluoro.

In one embodiment, oligomeric compounds are “hemimer oligomeric compounds” wherein chemical modifications to sugar moieties and/or internucleoside linkage distinguish a region of subunits at the 5′ terminus from a region of subunits at the 3′ terminus of the oligomeric compound.

In certain embodiments, oligomeric compounds contain at least one region modified so as to confer increased resistance to nuclease degradation, increased cellular uptake, and/or increased binding affinity for the target nucleic acid. An additional region of the oligomeric compound can, for example, contain a different modification, and in some cases may serve as a substrate for enzymes capable of cleaving RNA:DNA or RNA:RNA hybrids. By way of example, an oligomeric compound can be designed to comprise a region that serves as a substrate for RNase H. RNase H is a cellular endonuclease which cleaves the RNA strand of an RNA:DNA duplex. Activation of RNase H by an oligomeric compound having a cleavage region, therefore, results in cleavage of the RNA target, thereby enhancing the efficiency of the oligomeric compound. Alternatively, the binding affinity of the oligomeric compound for its target nucleic acid can be varied along the length of the oligomeric compound by including regions of chemically modified nucleosides which have exhibit either increased or decreased affinity as compared to the other regions. Consequently, comparable results can often be obtained with shorter oligomeric compounds having substrate regions when chimeras are used, compared to for example phosphorothioate deoxyoligonucleotides hybridizing to the same target region.

In certain embodiments, oligomeric compounds of the invention can be formed as composite structures of two or more oligonucleotides, oligonucleotide mimics, oligonucleotide analogs, oligonucleosides and/or oligonucleoside mimetics as described above. Such oligomeric compounds have also been referred to in the art as hybrids, hemimers, gapmers or inverted gapmers. Representative U.S. patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

In another aspect of the oligomeric compound there is a “gap-disabled” motif (also referred to as “gap-ablated motif”). In the gap-disabled motif, the internal region is interrupted by a chemical modification distinct from that of the internal region. The wing regions can be uniformly sized or differentially sized as also described above. Examples of gap-disabled motifs are as follows: 5′MMMMMMmmmMMMmmmmMMMM3′; 5′MMMMmmmmmmMmmmmmmmMM3′; 5′MMmmmmmmmmmmMMMmmmMM3′; wherein “m” represents one sugar modification and “M” represents a different sugar modification

As used in the present invention the term “alternating motif” is meant to include a contiguous sequence of nucleosides comprising two different nucleosides that alternate for essentially the entire sequence of the oligomeric compound. The pattern of alternation can be described by the formula: 5′-A(-L-B-L-A)n(-L-B)nn-3′ where A and B are nucleosides differentiated by having at least different sugar groups, each L is an internucleoside linking group, nn is 0 or 1 and n is from about 7 to about 11. This permits alternating oligomeric compounds from about 17 to about 24 nucleosides in length. This length range is not meant to be limiting as longer and shorter oligomeric compounds are also amenable to the present invention. This formula also allows for even and odd lengths for alternating oligomeric compounds wherein the 3′ and 5′-terminal nucleosides are the same (odd) or different (even). These alternating oligomeric compounds may comprise phosphodiester internucleotide linkages, phosphorothioate internucleotide linkages, or a combination of phosphodiester and phosphorothioate internucleotide linkages.

The “A” and “B” nucleosides comprising alternating oligomeric compounds of the present invention are differentiated from each other by having at least different sugar moieties. Each of the A and B nucleosides has a modified sugar moiety selected from β-D-ribonucleosides, β-D-deoxyribonucleosides, 2′-modified nucleosides (such 2′-modified nucleosides may include 2′-MOE, 2′-fluoro, and 2′-O—CH3, among others), and bicyclic sugar modified nucleosides. The alternating motif is independent from the nucleobase sequence and the internucleoside linkages. The internucleoside linkage can vary at each position or at particular selected positions or can be uniform or alternating throughout the oligomeric compound.

As used in the present invention the term “fully modified motif” is meant to include a contiguous sequence of sugar modified nucleosides wherein essentially each nucleoside is modified to have the same modified sugar moiety.

As used in the present invention the term “hemimer motif” is meant to include a sequence of nucleosides that have uniform sugar moieties (identical sugars, modified or unmodified) and wherein one of the 5′-end or the 3′-end has a sequence of from 2 to 12 nucleosides that are sugar modified nucleosides that are different from the other nucleosides in the hemimer modified oligomeric compound. An example of a typical hemimer is an oligomeric compound comprising 13-D-ribonucleosides or β-D-deoxyribonucleosides that have a sequence of sugar modified nucleosides at one of the termini. One hemimer motif includes a sequence of β-D-ribonucleosides or β-D-deoxyribonucleosides having from 2-12 sugar modified nucleosides located at one of the termini. Another hemimer motif includes a sequence of β-D-ribonucleosides or β-D-deoxyribonucleosides having from 2-6 sugar modified nucleosides located at one of the termini with from 2-4 being suitable. In a preferred embodiment of the invention, the oligomeric compound comprises a region of 2′-MOE modified nucleotides and a region of β-D-deoxyribonucleosides. In one embodiment, the β-D-deoxyribonucleosides comprise less than 13 contiguous nucleotides within the oligomeric compound. These hemimer oligomeric compounds may comprise phosphodiester internucleotide linkages, phosphorothioate internucleotide linkages, or a combination of phosphodiester and phosphorothioate internucleotide linkages.

As used in the present invention the term “blockmer motif” is meant to include a sequence of nucleosides that have uniform sugars (identical sugars, modified or unmodified) that is internally interrupted by a block of sugar modified nucleosides that are uniformly modified and wherein the modification is different from the other nucleosides. More generally, oligomeric compounds having a blockmer motif comprise a sequence of β-D-ribonucleosides or β-D-deoxyribonucleosides having one internal block of from 2 to 6, or from 2 to 4 sugar modified nucleosides. The internal block region can be at any position within the oligomeric compound as long as it is not at one of the termini which would then make it a hemimer. The base sequence and internucleoside linkages can vary at any position within a blockmer motif.

Nucleotides, both native and modified, have a certain conformational geometry which affects their hybridization and affinity properties. The terms used to describe the conformational geometry of homoduplex nucleic acids are “A Form” for RNA and “B Form” for DNA. The respective conformational geometry for RNA and DNA duplexes was determined from X-ray diffraction analysis of nucleic acid fibers (Arnott and Hukins, Biochem. Biophys. Res. Comm., 1970, 47, 1504.) In general, RNA:RNA duplexes are more stable and have higher melting temperatures (Tm's) than DNA:DNA duplexes (Sanger et al., Principles of Nucleic Acid Structure, 1984, Springer-Verlag; New York, N.Y.; Lesnik et al., Biochemistry, 1995, 34, 10807-10815; Conte et al., Nucleic Acids Res., 1997, 25, 2627-2634). The increased stability of RNA has been attributed to several structural features, most notably the improved base stacking interactions that result from an A-form geometry (Searle et al., Nucleic Acids Res., 1993, 21, 2051-2056). The presence of the 2′ hydroxyl in RNA biases the sugar toward a C3′ endo pucker, i.e., also designated as Northern pucker, which causes the duplex to favor the A-form geometry. In addition, the 2′ hydroxyl groups of RNA can form a network of water mediated hydrogen bonds that help stabilize the RNA duplex (Egli et al., Biochemistry, 1996, 35, 8489-8494). On the other hand, deoxy nucleic acids prefer a C2′ endo sugar pucker, i.e., also known as Southern pucker, which is thought to impart a less stable B-form geometry (Sanger, W. (1984) Principles of Nucleic Acid Structure, Springer-Verlag, New York, N.Y.). As used herein, B-form geometry is inclusive of both C2′-endo pucker and O4′-endo pucker. This is consistent with Berger, et. al., Nucleic Acids Research, 1998, 26, 2473-2480, who pointed out that in considering the furanose conformations which give rise to B-form duplexes consideration should also be given to a O4′-endo pucker contribution.

DNA:RNA hybrid duplexes, however, are usually less stable than pure RNA:RNA duplexes, and depending on their sequence may be either more or less stable than DNA:DNA duplexes (Searle et al., Nucleic Acids Res., 1993, 21, 2051-2056). The structure of a hybrid duplex is intermediate between A- and B-form geometries, which may result in poor stacking interactions (Lane et al., Eur. J. Biochem., 1993, 215, 297-306; Fedoroff et al., J. Mol. Biol., 1993, 233, 509-523; Gonzalez et al., Biochemistry, 1995, 34, 4969-4982; Horton et al., J. Mol. Biol., 1996, 264, 521-533). The stability of the duplex formed between a target RNA and a synthetic sequence is central to therapies such as, but not limited to, antisense mechanisms, including RNase H-mediated and RNA interference mechanisms, as these mechanisms involved the hybridization of a synthetic sequence strand to an RNA target strand. In the case of RNase H, effective inhibition of the mRNA requires that the antisense sequence achieve at least a threshold of hybridization.

One routinely used method of modifying the sugar puckering is the substitution of the sugar at the 2′-position with a substituent group that influences the sugar geometry. The influence on ring conformation is dependent on the nature of the substituent at the 2′-position. A number of different substituents have been studied to determine their sugar puckering effect. For example, 2′-halogens have been studied showing that the 2′-fluoro derivative exhibits the largest population (65%) of the C3′-endo form, and the 2′-iodo exhibits the lowest population (7%). The populations of adenosine (2′-OH) versus deoxyadenosine (2′-H) are 36% and 19%, respectively. Furthermore, the effect of the 2′-fluoro group of adenosine dimers (2′-deoxy-2′-fluoroadenosine-2′-deoxy-2′-fluoro-adenosine) is also correlated to the stabilization of the stacked conformation.

As expected, the relative duplex stability can be enhanced by replacement of 2′-OH groups with 2′-F groups thereby increasing the C3′-endo population. It is assumed that the highly polar nature of the 2′-F bond and the extreme preference for C3′-endo puckering may stabilize the stacked conformation in an A-form duplex. Data from UV hypochromicity, circular dichroism, and 1H NMR also indicate that the degree of stacking decreases as the electronegativity of the halo substituent decreases. Furthermore, steric bulk at the 2′-position of the sugar moiety is better accommodated in an A-form duplex than a B-form duplex. Thus, a 2′-substituent on the 3′-terminus of a dinucleoside monophosphate is thought to exert a number of effects on the stacking conformation: steric repulsion, furanose puckering preference, electrostatic repulsion, hydrophobic attraction, and hydrogen bonding capabilities. These substituent effects are thought to be determined by the molecular size, electronegativity, and hydrophobicity of the substituent. Melting temperatures of complementary strands is also increased with the 2′-substituted adenosine diphosphates. It is not clear whether the 3′-endo preference of the conformation or the presence of the substituent is responsible for the increased binding. However, greater overlap of adjacent bases (stacking) can be achieved with the 3′-endo conformation.

Nucleoside conformation is influenced by various factors including substitution at the 2′, 3′ or 4′-positions of the pentofuranosyl sugar. Electronegative substituents generally prefer the axial positions, while sterically demanding substituents generally prefer the equatorial positions (Principles of Nucleic Acid Structure, Wolfgang Sanger, 1984, Springer-Verlag.) Modification of the 2′ position to favor the 3′-endo conformation can be achieved while maintaining the 2′-OH as a recognition element (Gallo et al., Tetrahedron (2001), 57, 5707-5713. Harry-O′ kuru et al., J. Org. Chem., (1997), 62(6), 1754-1759 and Tang et al., J. Org. Chem. (1999), 64, 747-754.) Alternatively, preference for the 3′-endo conformation can be achieved by deletion of the 2′-OH as exemplified by 2′ deoxy-2′F-nucleosides (Kawasaki et al., J. Med. Chem. (1993), 36, 831-841), which adopts the 3′-endo conformation positioning the electronegative fluorine atom in the axial position. Other modifications of the ribose ring, for example substitution at the 4′-position to give 4′-F modified nucleosides (Guillerm et al., Bioorganic and Medicinal Chemistry Letters (1995), 5, 1455-1460 and Owen et al., J. Org. Chem. (1976), 41, 3010-3017), or for example modification to yield methanocarba nucleoside analogs (Jacobson et al., J. Med. Chem. Lett. (2000), 43, 2196-2203 and Lee et al., Bioorganic and Medicinal Chemistry Letters (2001), 11, 1333-1337) also induce preference for the 3′-endo conformation.

In one aspect of the present invention oligomeric compounds include nucleosides synthetically modified to induce a 3′-endo sugar conformation. A nucleoside can incorporate synthetic modifications of the heterocyclic base, the sugar moiety or both to induce a desired 3′-endo sugar conformation. These modified nucleosides are used to mimic RNA-like nucleosides so that particular properties of an oligomeric compound can be enhanced while maintaining the desirable 3′-endo conformational geometry. Properties that are enhanced by using more stable 3′-endo nucleosides include but are not limited to modulation of pharmacokinetic properties through modification of protein binding, protein off-rate, absorption and clearance; modulation of nuclease stability as well as chemical stability; modulation of the binding affinity and specificity of the oligomeric compound (affinity and specificity for enzymes as well as for complementary sequences); and increasing efficacy of RNA cleavage.

The conformation of modified nucleosides and their oligomers can be estimated by various methods such as molecular dynamics calculations, nuclear magnetic resonance spectroscopy and CD measurements. Hence, modifications predicted to induce RNA-like conformations (A-form duplex geometry in an oligomeric context), are useful in the oligomeric compounds of the present invention. The synthesis of modified nucleosides amenable to the present invention are known in the art (see for example, Chemistry of Nucleosides and Nucleotides Vol 1-3, ed. Leroy B. Townsend, 1988, Plenum Press.)

2. Certain Alternating Regions

In certain embodiments, oligonucleotides of the present invention comprise one or more regions of alternating modifications. In certain embodiments, oligonucleotides comprise one or more regions of alternating nucleoside modifications. In certain embodiments, oligonucleotides comprise one or more regions of alternating linkage modifications. In certain embodiments, oligonucleotides comprise one or more regions of alternating nucleoside and linkage modifications.

In certain embodiments, oligonucleotides of the present invention comprise one or more regions of alternating tetrahydropyran nucleosides and non-tetrahydropyran modified nucleosides. In certain such embodiments, such regions of alternating tetrahydropyran nucleosides and non-tetrahydropyran modified nucleosides also comprise alternating linkages.

In certain embodiments, oligomeric compounds of the present invention comprise a motif of motif I:

T1-(Nu₁)ₙ₁-(Nu₂)ₙ₂(Nu₃)ₙ₃(Nu₄)ₙ₄-(Nu₅)ₙ₅-T₂, wherein:
Nu₁, Nu₃, and Nuₛ are each independently modified or unmodified nucleosides or nucleoside analogs other than tetrahydropyran nucleoside analogs;
Nu₂ and Nu₄ are each independently tetrahydropyran nucleoside analogs of Formula I;
each of n1 and n5 is, independently from 0 to 3;
the sum of n2 plus n4 is between 10 and 25;
n3 is from 0 and 5; and
each T₁ and T₂ is, independently, H, a hydroxyl protecting group, an optionally linked conjugate group or a capping group.
   In certain such embodiments, the sum of n2 and n4 is 13 or 14; n1 is 2; n3 is 2 or 3; and n5 is 2. In certain such embodiments, formula I is selected from Table A or Table B.

TABLE A

| n1 | n2 | n3 | n4 | n5 |
|---|---|---|---|---|
| 2 | 16 | 0 | 0 | 2 |
| 2 | 2 | 3 | 11 | 2 |
| 2 | 5 | 3 | 8 | 2 |
| 2 | 8 | 3 | 5 | 2 |
| 2 | 11 | 3 | 2 | 2 |
| 2 | 9 | 3 | 4 | 2 |
| 2 | 10 | 3 | 3 | 2 |
| 2 | 3 | 3 | 10 | 2 |
| 2 | 4 | 3 | 9 | 2 |
| 2 | 6 | 3 | 7 | 2 |
| 2 | 7 | 3 | 6 | 2 |
| 2 | 8 | 6 | 2 | 2 |
| 2 | 2 | 2 | 12 | 2 |
| 2 | 3 | 2 | 11 | 2 |
| 2 | 4 | 2 | 10 | 2 |
| 2 | 5 | 2 | 9 | 2 |
| 2 | 6 | 2 | 8 | 2 |
| 2 | 7 | 2 | 7 | 2 |
| 2 | 8 | 2 | 6 | 2 |
| 2 | 9 | 2 | 5 | 2 |
| 2 | 10 | 2 | 4 | 2 |
| 2 | 11 | 2 | 3 | 2 |
| 2 | 12 | 2 | 2 | 2 |

TABLE B

| n1 | n2 | n3 | n4 | n5 |
|---|---|---|---|---|
| 2 | 2 | 3 | 11 | 2 |
| 2 | 5 | 3 | 8 | 2 |
| 2 | 8 | 3 | 5 | 2 |
| 2 | 11 | 3 | 2 | 2 |
| 2 | 9 | 3 | 4 | 2 |
| 2 | 10 | 3 | 3 | 2 |
| 2 | 3 | 3 | 10 | 2 |
| 2 | 4 | 3 | 9 | 2 |
| 2 | 6 | 3 | 7 | 2 |
| 2 | 7 | 3 | 6 | 2 |
| 2 | 8 | 6 | 2 | 2 |
| 2 | 2 | 2 | 12 | 2 |
| 2 | 3 | 2 | 11 | 2 |
| 2 | 4 | 2 | 10 | 2 |
| 2 | 5 | 2 | 9 | 2 |
| 2 | 6 | 2 | 8 | 2 |
| 2 | 7 | 2 | 7 | 2 |
| 2 | 8 | 2 | 6 | 2 |
| 2 | 9 | 2 | 5 | 2 |
| 2 | 10 | 2 | 4 | 2 |
| 2 | 11 | 2 | 3 | 2 |
| 2 | 12 | 2 | 2 | 2 |

Tables A and B are intended to illustrate, but not to limit the present invention. The oligomeric compounds depicted in Tables A and B each comprise 20 nucleosides. Oligomeric compounds comprising more or fewer nucleosides can easily by designed by selecting different numbers of nucleosides for one or more of n1-n5.

In certain embodiments, the sum of n₂ and n₄ is 13. In certain embodiments, the sum of n₂ and n₄ is 14. In certain embodiments, the sum of n₂ and n₄ is 15. In certain embodiments, the sum of n₂ and n₄ is 16. In certain embodiments, the sum of n₂ and n₄ is 17. In certain embodiments, the sum of n₂ and n₄ is 18.

In certain embodiments, n₁, n₂, and n₃ are each, independently, from 1 to 3. In certain embodiments, nₜ, n₂, and n₃ are each, independently, from 2 to 3. In certain embodiments, n₁ is 1 or 2; n₂ is 2 or 3; and n₃ is 1 or 2. In certain embodiments, n₁ is 2; n₃ is 2 or 3; and n₅ is 2. In certain embodiments, n₁ is 2; n₃ is 3; and n₅ is 2. In certain embodiments, n₁ is 2; n₃ is 2; and n₅ is 2.

In certain embodiments, a modified oligonucleotide consists of 20 linked nucleosides. In certain such embodiments, the sum of n₂ and n₄ is 13; n₁ is 2; n₃ is 3; and n₅ is 2. In certain such embodiments, the sum of n₂ and n₄ is 14; n₁ is 2; n₃ is 2; and n₅ is 2.

In certain embodiments, a modified oligonucleotide consists of 21 linked nucleosides. In certain such embodiments, the sum of n₂ and n₄ is 14; n₁ is 2; n₃ is 3; and n₅ is 2. In certain such embodiments, the sum of n₂ and n₄ is 15; n₁ is 2; n₃ is 2; and n₅ is 2.

In certain embodiments, a modified oligonucleotide consists of 22 linked nucleosides. In certain such embodiments, the sum of n₂ and n₄ is 15; n₁ is 2; n₃ is 3; and n₅ is 2. In certain such embodiments, the sum of n₂ and n₄ is 16; n₁ is 2; n₃ is 2; and n₅ is 2.

In certain embodiments, a modified oligonucleotide consists of 23 linked nucleosides. In certain such embodiments, the sum of n₂ and n₄ is 16; n₁ is 2; n₃ is 3; and n₅ is 2. In certain such embodiments, the sum of n₂ and n₄ is 17; n₁ is 2; n₃ is 2; and n₅ is 2.

In certain embodiments, a modified oligonucleotide consists of 24 linked nucleosides. In certain such embodiments, the sum of n₂ and n₄ is 17; n₁ is 2; n₃ is 3; and n₅ is 2. In certain such embodiments, the sum of n₂ and n₄ is 18; n₁ is 2; n₃ is 2; and n₅ is 2.

In certain embodiments, a modified oligonucleotide consists of 22 linked nucleosides; n₁ is 2; n₂ is 9; n₃ is 3; n₄ is 6; n₅ is 2; Nu₁ is O—(CH₂)₂—OCH₃; Nu₃ is O—(CH₂)₂—OCH₃; and Nu₅ O—(CH₂)₂—OCH₃.

In certain embodiments, a modified oligonucleotide consists of 22 linked nucleosides; n₁ is 2; n₂ is 9; n₃ is 3; n₄ is 6; n₅ is 2; Nu₁ is O—(CH₂)₂—OCH₃; Nu₃ is O—(CH₂)₂—OCH₃; Nu₅ O—(CH₂)₂—OCH₃; and each internucleoside linkage is a phosphorothioate linkage.

In certain embodiments, a modified oligonucleotide consists of 22 linked nucleosides; n₁ is 2; n₂ is 9; n₃ is 3; n₄ is 6; n₅ is 2; Nu₁ is O—(CH₂)₂—OCH₃; Nu₃ is O—(CH₂)₂—OCH₃; Nu_{s O—(CH}₂); internucleoside linkage is a phosphorothioate linkage.

In certain embodiments, a modified oligonucleotide consists of 22 linked nucleosides; has the nucleobase sequence of SEQ ID NO: 4; n₁ is 2; n₂ is 9; n₃ is 3; n₄ is 6; n₅ is 2; Nu₁ is O—(CH₂)₂—OCH₃; Nu₃ is O—(CH₂)₂—OCH₃; Nuₛ O—(CH₂); each internucleoside linkage is a phosphorothioate linkage; the cytosine at nucleobase 13 is a 5-methylcytosine; and the cytosine at nucleobase 21 is a 5-methylcytosine (referred to herein as anti-miR-223-1).

In certain embodiments, one or more alternating regions in an alternating motif include more than a single nucleoside of a type. For example, oligomeric compounds of the present invention may include one or more regions of any of the following nucleoside motifs:
Nu₁ Nu₁ Nu₂ Nu₂ Nu₁ Nu₁;
Nu₁ Nu₂ Nu₂ Nu₁ Nu₂ Nu₂;
Nu₁ Nu₁ Nu₂ Nu₁ Nu₁ Nu₂;
Nu₁ Nu₂Nu₂Nu₁ Nu₂ Nu₁ Nu₁ Nu₂ Nu₂;
Nu₁ Nu₂ Nu₁ Nu₂ Nu₁ Nu₁;
Nu₁ Nu₁ Nu₂ Nu₁ Nu₂Nu₁ Nu₂;
Nu₁ Nu₂ Nu₁ Nu₂ Nu₁ Nu₁;
Nu₁ Nu₂Nu₂Nu₁ Nu₁ Nu₂Nu₂Nu₁ Nu₁ Nu₁ Nu₂ Nu₁ Nu₁;
Nu₂ Nu₁ Nu₂ Nu₂ Nu₁ Nu₁ Nu₂Nu₂Nu₁Nu₂Nu₁Nu₂Nu₁ Nu₁; or
Nu₁ Nu₂Nu₁ Nu₂ Nu₂ Nu₁ Nu₁ Nu₂Nu₂Nu₁ Nu₂ Nu₁ Nu₂ Nu₁ Nu₁;
   wherein Nu₁ is a nucleoside of a first type and Nu₂ is a nucleoside of a second type. In certain embodiments, one of Nu₁ and Nu₂ is a tetrahydropyran nucleoside and the other of Nu₁ and Nu₂ is a non-tetrahydropyran nucleoside selected from: a 2′-F modified nucleoside, a 2′-OMe modified nucleoside, BNA, a 2′-MOE modified nucleoside, and an unmodified DNA or RNA nucleoside.

Solely to illustrate and not to limit the present invention, other examples of motifs include, but are not limited to:

N_{f}N_{d}N_{f}NaN_{d}N_{d}N_{f}N_{f}N_{d}N_{d}N_{f}N_{d}N_{d}N_{d}N_{f}N_{f}
N_{f}N_{d}N_{f}N_{d}N_{d}N_{f}N_{f}N_{d}N_{d}N_{f}N_{d}N_{f}N_{d}N_{f}N_{d}NₑNₑ
N_{f}N_{f}N_{f}N_{d}N_{d}N_{f}N_{d}N_{d}N_{d}N_{f}N_{d}N_{d}N_{d}N_{f}N_{d}N_{f}N_{f}
N_{f}N_{f}N_{f}N_{d}N_{d}N_{f}N_{f}N_{f}N_{d}N_{d}N_{d}N_{f}N_{f}N_{f}N_{d}N_{f}N_{f}
N_{f}N_{d}N_{f}N_{d}N_{d}N_{f}N_{f}N_{d}N_{d}N_{f}N_{d}N_{f}N_{d}N_{f}N_{f}N_{f}N_{f}
N_{f}N_{d}N_{f}N_{d}N_{d}N_{f}N_{f}N_{d}N_{d}N_{f}N_{d}N_{f}N_{d}N_{f}N_{f}N_{d}N_{d}
N_{d}N_{d}N_{f}N_{f}N_{f}N_{f}N_{f}N_{f}N_{f}N_{d}N_{f}N_{f}N_{f}N_{f}N_{f}N_{d}N_{d}
NₑN_{f}N_{f}N_{f}N_{f}N_{f}N_{f}N_{f}N_{f}N_{f}N_{f}N_{f}N_{f}N_{f}N_{f}N_{f}Nₑ
NₑN_{f}N_{f}N_{f}N_{f}NₑNₑNₑN_{f}N_{f}N_{f}N_{f}N_{f}N_{f}N_{f}N_{f}Nₑ
N_{f}N_{d}N_{f}NₑN_{d}N_{f}N_{f}N_{d}NₑN_{f}N_{d}N_{f}NₑN_{f}N_{d}N_{f}N_{f} and
NₑN_{d}N_{f}N_{d}N_{d}N_{f}N_{f}N_{d}N_{d}N_{f}N_{d}N_{f}N_{d}N_{f}N_{d}NₑNₑ
where N is a nucleoside having any nucleobase, subscript e is 2′-MOE; d is unmodified DNA; and f is a tetrahydropyran, for example:

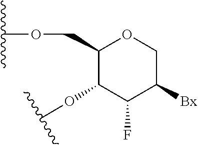

C. Oligomeric Compounds

In certain embodiments, the present invention provides oligomeric compounds. In certain embodiments, oligomeric compounds comprise an oligonucleotide. In certain embodiments, an oligomeric compound comprises an oligonucleotide and one or more conjugate and/or terminal groups. Such conjugate and/or terminal groups may be added to oligonucleotides having any of the chemical motifs discussed above. Thus, for example, an oligomeric compound comprising an oligonucleotide having region of alternating nucleosides may comprise a terminal group.

1. Certain Conjugate Groups

In certain embodiments, oligomeric compounds are modified by attachment of one or more conjugate groups. In general, conjugate groups modify one or more properties of the attached oligomeric compound including but not limited to pharmacodynamics, pharmacokinetics, stability, binding, absorption, cellular distribution, cellular uptake, charge and clearance. Conjugate groups are routinely used in the chemical arts and are linked directly or via an optional conjugate linking moiety or conjugate linking group to a parent compound such as an oligomeric compound, such as an oligonucleotide. Conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes. Certain conjugate groups have been described previously, for example: cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-5-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937).

In certain embodiments, a conjugate group comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130.

Representative U.S. patents that teach the preparation of oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

In certain embodiments, conjugate groups are directly attached to oligonucleotides in oligomeric compounds. In certain embodiments, conjugate groups are attached to oligonucleotides by a conjugate linking group. In certain such embodiments, conjugate linking groups, including, but not limited to, bifunctional linking moieties such as those known in the art are amenable to the compounds provided herein. Conjugate linking groups are useful for attachment of conjugate groups, such as chemical stabilizing groups, functional groups, reporter groups and other groups to selective sites in a parent compound such as for example an oligomeric compound. In general a bifunctional linking moiety comprises a hydrocarbyl moiety having two functional groups. One of the functional groups is selected to bind to a parent molecule or compound of interest and the other is selected to bind essentially any selected group such as chemical functional group or a conjugate group. In some embodiments, the conjugate linker comprises a chain structure or an oligomer of repeating units such as ethylene glycol or amino acid units. Examples of functional groups that are routinely used in a bifunctional linking moiety include, but are not limited to, electrophiles for reacting with nucleophilic groups and nucleophiles for reacting with electrophilic groups. In some embodiments, bifunctional linking moieties include amino, hydroxyl, carboxylic acid, thiol, unsaturations (e.g., double or triple bonds), and the like.

Some nonlimiting examples of conjugate linking moieties include pyrrolidine, 8-amino-3,6-dioxaoctanoic acid (ADO), succinimidyl 4-(N-maleimidomethyl)cyclohexane-1-carboxylate (SMCC) and 6-aminohexanoic acid (AHEX or AHA). Other linking groups include, but are not limited to, substituted C1-C10 alkyl, substituted or unsubstituted C2-C10 alkenyl or substituted or unsubstituted C2-C10 alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

Conjugate groups may be attached to either or both ends of an oligonucleotide (terminal conjugate groups) and/or at any internal position.

2. Terminal Groups

In certain embodiments, oligomeric compounds comprise terminal groups at one or both ends. In certain embodiments, a terminal group may comprise any of the conjugate groups discussed above. In certain embodiments, terminal groups may comprise additional nucleosides and/or inverted abasic nucleosides. In certain embodiments, a terminal group is a stabilizing group.

In certain embodiments, oligomeric compounds comprise one or more terminal stabilizing group that enhances properties such as for example nuclease stability. Included in stabilizing groups are cap structures. The terms “cap structure” or “terminal cap moiety,” as used herein, refer to chemical modifications, which can be attached to one or both of the termini of an oligomeric compound. These terminal modifications protect the oligomeric compounds having terminal nucleic acid moieties from exonuclease degradation, and can help in delivery and/or localization within a cell. The cap can be present at the 5′-terminus (5′-cap) or at the 3′-terminus (3′-cap) or can be present on both termini. In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl riboucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270).

Particularly suitable 3′-cap structures of the present invention include, for example 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxy-pentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Tyer, 1993, Tetrahedron 49, 1925 and Published U.S. Patent Application Publication No. US 2005/0020525 published on Jan. 27, 2005). Further 3′ and 5′-stabilizing groups that can be used to cap one or both ends of an oligomeric compound to impart nuclease stability include those disclosed in WO 03/004602.

3. Terminal-Group Nucleosides

In certain embodiments, one or more additional nucleosides is added to one or both terminal ends of an oligonucleotide of an oligomeric compound. Such additional terminal nucleosides are referred to herein as terminal-group nucleosides. In a double-stranded compound, such terminal-group nucleosides are terminal (3′ and/or 5′) overhangs. In the setting of double-stranded antisense compounds, such terminal-group nucleosides may or may not be complementary to a target nucleic acid.

In a single-stranded antisense oligomeric compound, terminal-group nucleosides are typically non-hybridizing. The terminal-group nucleosides are typically added to provide a desired property other than hybridization with target nucleic acid. Nonetheless, the target may have complementary bases at the positions corresponding with the terminal-group nucleosides. Whether by design or accident, such complementarity of one or more terminal-group nucleosides does not alter their designation as terminal-group nucleosides. In certain embodiments, the bases of terminal-group nucleosides are each selected from adenine (A), uracil (U), guanine (G), cytosine (C), thymine (T), and analogs thereof. In certain embodiments, the bases of terminal-group nucleosides are each selected from adenine (A), uracil (U), guanine (G), cytosine (C), and thymine (T). In certain embodiments, the bases of terminal-group nucleosides are each selected from adenine (A), uracil (U), and thymine (T). In certain embodiments, the bases of terminal-group nucleosides are each selected from adenine (A) and thymine (T). In certain embodiments, the bases of terminal-group nucleosides are each adenine (A). In certain embodiments, the bases of terminal-group nucleosides are each thymine (T). In certain embodiments, the bases of terminal-group nucleosides are each uracil (U). In certain embodiments, the bases of terminal-group nucleosides are each cytosine (C). In certain embodiments, the bases of terminal-group nucleosides are each guanine (G).

In certain embodiments, terminal-group nucleosides are sugar modified. In certain such embodiments, such additional nucleosides are 2′-modified. In certain embodiments, the 2% modification of terminal-group nucleosides are selected from among 2′-F, 2′-OMe, and 2′-MOE. In certain embodiments, terminal-group nucleosides are 2′-MOE modified. In certain embodiments, terminal-group nucleosides comprise 2′-MOE sugar moieties and adenine nucleobases (2′-MOE A nucleosides). In certain embodiments, terminal-group nucleosides comprise 2′-MOE sugar moieties and uracil nucleobases (2′-MOE U nucleosides). In certain embodiments, terminal-group nucleosides comprises 2′-MOE sugar moieties and guanine nucleobases (2′-MOE G nucleosides). In certain embodiments, terminal-group nucleosides comprises 2′-MOE sugar moieties and thymine nucleobases (2′-MOE T nucleosides). In certain embodiments, terminal-group nucleosides comprises 2′-MOE sugar moieties and cytosine nucleobases (2′-MOE C nucleosides).

In certain embodiments, terminal-group nucleosides comprise bicyclic sugar moieties. In certain such embodiments, terminal-group nucleosides comprise LNA sugar moieties. In certain embodiments, terminal-group nucleosides comprise LNA sugar moieties and adenine nucleobases (LNA A nucleosides). In certain embodiments, terminal-group nucleosides comprise LNA sugar moieties and uracil nucleobases (LNA nucleosides). In certain embodiments, terminal-group nucleosides comprise LNA sugar moieties and guanine nucleobases (LNA G nucleosides). In certain embodiments, terminal-group nucleosides comprise LNA sugar moieties and thymine nucleobases (LNA T nucleosides). In certain embodiments, terminal-group nucleosides comprise LNA sugar moieties and cytosine nucleobases (LNA C nucleosides).

In certain embodiments, oligomeric compounds comprise 1-4 terminal-group nucleosides at the 3′ end of the oligomeric compound. In certain embodiments, oligomeric compounds comprise 1-3 terminal-group nucleosides at the 3′ end of the oligomeric compound. In certain embodiments, oligomeric compounds comprise 1-2 terminal-group nucleosides at the 3′ end of the oligomeric compound. In certain embodiments, oligomeric compounds comprise 2 terminal-group nucleosides at the 3′ end of the oligomeric compound. In certain embodiments, oligomeric compounds comprise 1 terminal-group nucleoside at the 3′ end of the oligomeric compound. In certain embodiments having two or more terminal-group nucleosides, the two or more terminal-group nucleosides all have the same modification type and the same base. In certain embodiments having two or more terminal-group nucleosides, the terminal-group nucleosides differ from one another by modification and/or base.

In certain embodiments, oligomeric compounds comprise a 3′-terminal group comprising 2 terminal-group nucleosides, wherein each terminal group nucleoside is a 2′-MOE T. In certain embodiments, oligomeric compounds comprise a 3′-terminal group comprising 2 terminal-group nucleosides, wherein each terminal group nucleoside is a 2′-MOE A. In certain embodiments, oligomeric compounds comprise a 3′-terminal group comprising 2 terminal-group nucleosides, wherein each terminal group nucleoside is a 2′-MOE U. In certain embodiments, oligomeric compounds comprise a 3′-terminal group comprising 2 terminal-group nucleosides, wherein each terminal group nucleoside is a 2′-MOE C. In certain embodiments, oligomeric compounds comprise a 3′-terminal group comprising 2 terminal-group nucleosides, wherein each terminal group nucleoside is a 2′-MOE G.

In certain embodiments, oligomeric compounds comprise a 3′-terminal group comprising 2 terminal-group nucleosides, wherein each terminal group nucleoside is a LNA T. In certain embodiments, oligomeric compounds comprise a 3′-terminal group comprising 2 terminal-group nucleosides, wherein each terminal group nucleoside is a LNA A. In certain embodiments, oligomeric compounds comprise a 3′-terminal group comprising 2 terminal-group nucleosides, wherein each terminal group nucleoside is a LNA U. In certain embodiments, oligomeric compounds comprise a 3′-terminal group comprising 2 terminal-group nucleosides, wherein each terminal group nucleoside is a LNA C. In certain embodiments, oligomeric compounds comprise a 3′-terminal group comprising 2 terminal-group nucleosides, wherein each terminal group nucleoside is a LNA G.

D. Antisense Compounds

In certain embodiments, oligomeric compounds of the present invention are antisense compounds. In such embodiments, the oligomeric compound is complementary to a target nucleic acid. In certain embodiments, a target nucleic acid is an RNA. In certain embodiments, a target nucleic acid is a non-coding RNA. In certain embodiments, a target nucleic acid encodes a protein. In certain embodiments, a target nucleic acid is selected from a mRNA, a pre-mRNA, a microRNA, a non-coding RNA, including small non-coding RNA, and a promoter-directed RNA. In certain embodiments, oligomeric compounds are at least partially complementary to more than one target nucleic acid. For example, oligomeric compounds of the present invention may be microRNA mimics, which typically bind to multiple targets.

In certain embodiments, antisense compounds comprise a portion having a nucleobase sequence at least 70% complementary to the nucleobase sequence of a target nucleic acid. In certain embodiments, antisense compounds comprise a portion having a nucleobase sequence at least 80% complementary to the nucleobase sequence of a target nucleic acid. In certain embodiments, antisense compounds comprise a portion having a nucleobase sequence at least 90% complementary to the nucleobase sequence of a target nucleic acid. In certain embodiments, antisense compounds comprise a portion having a nucleobase sequence at least 95% complementary to the nucleobase sequence of a target nucleic acid. In certain embodiments, antisense compounds comprise a portion having a nucleobase sequence at least 98% complementary to the nucleobase sequence of a target nucleic acid. In certain embodiments, antisense compounds comprise a portion having a nucleobase sequence that is 100% complementary to the nucleobase sequence of a target nucleic acid. In certain embodiments, antisense compounds are at least 70%, 80%, 90%, 95%, 98%, or 100% complementary to the nucleobase sequence of a target nucleic acid over the entire length of the antisense compound.

Antisense mechanisms include any mechanism involving the hybridization of an oligomeric compound with target nucleic acid, wherein the hybridization results in a biological effect. In certain embodiments, such hybridization results in either target nucleic acid degradation or occupancy with concomitant inhibition or stimulation of the cellular machinery involving, for example, translation, transcription, splicing or polyadenylation of the target nucleic acid or of a nucleic acid with which the target nucleic acid may otherwise interact.

One type of antisense mechanism involving degradation of target RNA is RNase H mediated antisense. RNase H is a cellular endonuclease which cleaves the RNA strand of an RNA:DNA duplex. It is known in the art that single-stranded antisense compounds which are “DNA-like” elicit RNase H activity in mammalian cells. Activation of RNase H, therefore, results in cleavage of the RNA target, thereby greatly enhancing the efficiency of DNA-like oligonucleotide-mediated inhibition of gene expression.

Antisense mechanisms also include, without limitation RNAi mechanisms, which utilize the RISC pathway. Such RNAi mechanisms include, without limitation siRNA, ssRNA and microRNA mechanisms. Such mechanism include creation of a microRNA mimic and/or an anti-microRNA.

Antisense mechanisms also include, without limitation, mechanisms that hybridize or mimic non-coding RNA other than microRNA or mRNA. Such non-coding RNA includes, but is not limited to promoter-directed RNA and short and long RNA that effects transcription or translation of one or more nucleic acids.

In certain embodiments, antisense compounds specifically hybridize when there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

As used herein, “stringent hybridization conditions” or “stringent conditions” refers to conditions under which an antisense compound will hybridize to its target sequence, but to a minimal number of other sequences. Stringent conditions are sequence-dependent and will be different in different circumstances, and “stringent conditions” under which antisense compounds hybridize to a target sequence are determined by the nature and composition of the antisense compounds and the assays in which they are being investigated.

It is understood in the art that incorporation of nucleotide affinity modifications may allow for a greater number of mismatches compared to an unmodified compound. Similarly, certain oligonucleotide sequences may be more tolerant to mismatches than other oligonucleotide sequences. One of ordinary skill in the art is capable of determining an appropriate number of mismatches between oligonucleotides, or between an oligonucleotide and a target nucleic acid, such as by determining melting temperature (Tₘ). Tₘ or ΔTₘ can be calculated by techniques that are familiar to one of ordinary skill in the art. For example, techniques described in Freier et al. (Nucleic Acids Research, 1997, 25, 22: 4429-4443) allow one of ordinary skill in the art to evaluate nucleotide modifications for their ability to increase the melting temperature of an RNA:DNA duplex.

In certain embodiments, oligomeric compounds of the present invention are RNAi compounds. In certain embodiments, oligomeric compounds of the present invention are ssRNA compounds. In certain embodiments, oligomeric compounds of the present invention are paired with a second oligomeric compound to form an siRNA. In certain such embodiments, the second oligomeric compound is also an oligomeric compound of the present invention. In certain embodiments, the second oligomeric compound is any modified or unmodified nucleic acid. In certain embodiments, the oligomeric compound of the present invention is the antisense strand in an siRNA compound. In certain embodiments, the oligomeric compound of the present invention is the sense strand in an siRNA compound.

2. Oligomeric Compound Identity

In certain embodiments, a portion of an oligomeric compound is 100% identical to the nucleobase sequence of a microRNA, but the entire oligomeric compound is not fully identical to the microRNA. In certain such embodiments, the length of an oligomeric compound having a 100% identical portion is greater than the length of the microRNA. For example, a microRNA mimic consisting of 24 linked nucleosides, where the nucleobases at positions 1 through 23 are each identical to corresponding positions of a microRNA that is 23 nucleobases in length, has a 23 nucleoside portion that is 100% identical to the nucleobase sequence of the microRNA and has approximately 96% overall identity to the nucleobase sequence of the microRNA.

In certain embodiments, the nucleobase sequence of oligomeric compound is fully identical to the nucleobase sequence of a portion of a microRNA. For example, a single-stranded microRNA mimic consisting of 22 linked nucleosides, where the nucleobases of positions 1 through 22 are each identical to a corresponding position of a microRNA that is 23 nucleobases in length, is fully identical to a 22 nucleobase portion of the nucleobase sequence of the microRNA. Such a single-stranded microRNA mimic has approximately 96% overall identity to the nucleobase sequence of the entire microRNA, and has 100% identity to a 22 nucleobase portion of the microRNA.

microRNA Targets and Mimics

In certain embodiments, an antisense compound comprises a region comprising a nucleobase sequence having at least partial identity or complementarity to a microRNA sequence associated with an accession number from miRBase version 10.1 released December 2007 selected from:

MIMAT0000062, MIMAT0004481, MIMAT0000063, MIMAT0004482, MIMAT0000064, MIMAT0004483, MIMAT0000065, MIMAT0004484, MIMAT0000066, MIMAT0004485, MIMAT0000067, MIMAT0004486, MIMAT0004487, MIMAT0000414, MIMAT0004584, MIMAT0000415, MIMAT0004585, MIMAT0000416, MIMAT0000098, MIMAT0004512, MIMAT0000099, MIMAT0004513, MIMAT0000101, MIMAT0000102, MIMAT0004516, MIMAT0000103, MIMAT0004517, MIMAT0000680, MIMAT0004672, MIMAT0000104, MIMAT0000253, MIMAT0004555, MIMAT0000254, MIMAT0004556, MIMAT0000421, MIMAT0004590, MIMAT0005459, MIMAT0005458, MIMAT0005573, MIMAT0005572, MIMAT0005577, MIMAT0005576, MIMAT0005580, MIMAT0005583, MIMAT0005582, MIMAT0005584, MIMAT0005586, MIMAT0005588, MIMAT0005589, MIMAT0005591, MIMAT0005592, MIMAT0005593, MIMAT0000422, MIMAT0004591, MIMAT0004602, MIMAT0000443, MIMAT0000423, MIMAT0000423, MIMAT0004592, MIMAT0004603, MIMAT0000445, MIMAT0000444, MIMAT0000446, MIMAT0004604, MIMAT0000424, MIMAT0004548, MIMAT0004605, MIMAT0000242, MIMAT0000425, MIMAT0004593, MIMAT0000691, MIMAT0004680, MIMAT0000426, MIMAT0004594, MIMAT0000427, MIMAT0000770, MIMAT0000447, MIMAT0000428, MIMAT0004595, MIMAT0000758, MIMAT0004698, MIMAT0000448, MIMAT0004606, MIMAT0000429, MIMAT0000430, MIMAT0004607, MIMAT0004596, MIMAT0004552, MIMAT0000250, MIMAT0004597, MIMAT0000431, MIMAT0000432, MIMAT0004598, MIMAT0000434, MIMAT0000433, MIMAT0000435, MIMAT0004599, MIMAT0000436, MIMAT0004600, MIMAT0000437, MIMAT0004601, MIMAT0000449, MIMAT0004608, MIMAT0004766, MIMAT0002809, MIMAT0000251, MIMAT0004928, MIMAT0000243, MIMAT0004549, MIMAT0000759, MIMAT0004699, MIMAT0000450, MIMAT0004609, MIMAT0000451, MIMAT0004610, MIMAT0000757, MIMAT0004697, MIMAT0000438, MIMAT0000439, MIMAT0000439, MIMAT0000452, MIMAT0000453, MIMAT0000646, MIMAT0004658, MIMAT0000068, MIMAT0004488, MIMAT0000417, MIMAT0004586, MIMAT0000069, MIMAT0004489, MIMAT0004518, MIMAT0000070, MIMAT0000071, MIMAT0000256, MIMAT0000270, MIMAT0004558, MIMAT0000257, MIMAT0000258, MIMAT0004559, MIMAT0002821, MIMAT0000259, MIMAT0000260, MIMAT0000261, MIMAT0004560, MIMAT0000454, MIMAT0004611, MIMAT0000456, MIMAT0004612, MIMAT0000262, MIMAT0004561, MIMAT0004613, MIMAT0000457, MIMAT0000072, MIMAT0002891, MIMAT0001412, MIMAT0004751, MIMAT0000458, MIMAT0004929, MIMAT0000440, MIMAT0001618, MIMAT0000222, MIMAT0004543, MIMAT0000459, MIMAT0004614, MIMAT0002819, MIMAT0004767, MIMAT0000460, MIMAT0004671, MIMAT0000461, MIMAT0004615, MIMAT0000226, MIMAT0004562, MIMAT0001080, MIMAT0000227, MIMAT0000228, MIMAT0000232, MIMAT0000231, MIMAT0004563, MIMAT0000263, MIMAT0000073, MIMAT0004490, MIMAT0000074, MIMAT0004491, MIMAT0004492, MIMAT0000682, MIMAT0001620, MIMAT0000318, MIMAT0004571, MIMAT0000617, MIMAT0004657, MIMAT0002811, MIMAT0002810, MIMAT0000264, MIMAT0000265, MIMAT0000266, MIMAT0000462, MIMAT0000241, MIMAT0004960, MIMAT0000075, MIMAT0004493, MIMAT0001413, MIMAT0004752, MIMAT0000076, MIMAT0004494, MIMAT0000267, MIMAT0000268, MIMAT0000269, MIMAT0000271, MIMAT0004564, MIMAT0000272, MIMAT0000273, MIMAT0004959, MIMAT0000274, MIMAT0000275, MIMAT0004565, MIMAT0004566, MIMAT0004567, MIMAT0004675, MIMAT0000276, MIMAT0000077, MIMAT0004495, MIMAT0000277, MIMAT0004908, MIMAT0004915, MIMAT0000278, MIMAT0004568, MIMAT0000279, MIMAT0004569, MIMAT0000280, MIMAT0004570, MIMAT0000281, MIMAT0000078, MIMAT0004496, MIMAT0000418, MIMAT0004587, MIMAT0000080, MIMAT0000079, MIMAT0004497, MIMAT0000081, MIMAT0004498, MIMAT0000082, MIMAT0004499, MIMAT0004681, MIMAT0000083, MIMAT0004500, MIMAT0000084, MIMAT0004501, MIMAT0000419, MIMAT0004588, MIMAT0004502, MIMAT0000085, MIMAT0004679, MIMAT0000690, MIMAT0004450, MIMAT0004901, MIMAT0000687, MIMAT0002890, MIMAT0000086, MIMAT0004503, MIMAT0000100, MIMAT0004514, MIMAT0004515, MIMAT0000681, MIMAT0004673, MIMAT0004903, MIMAT0000688, MIMAT0004958, MIMAT0000684, MIMAT0000683, MIMAT0000715, MIMAT0000714, MIMAT0000717, MIMAT0000716, MIMAT0000718, MIMAT0004685, MIMAT0000087, MIMAT0000088, MIMAT0000420, MIMAT0004589, MIMAT0000244, MIMAT0004674, MIMAT0004550, MIMAT0000245, MIMAT0004551, MIMAT0000692, MIMAT0000693, MIMAT0000089, MIMAT0004504, MIMAT0000090, MIMAT0004505, MIMAT0000510, MIMAT0000755, MIMAT0004696, MIMAT0000762, MIMAT0000761, MIMAT0000771, MIMAT0000756, MIMAT0000752, MIMAT0001629, MIMAT0000751, MIMAT0004693, MIMAT0000760, MIMAT0004700, MIMAT0000765, MIMAT0004703, MIMAT0000754, MIMAT0004695, MIMAT0000763, MIMAT0004701, MIMAT0004702, MIMAT0000764, MIMAT0000091, MIMAT0004506, MIMAT0003301, MIMAT0004811, MIMAT0004692, MIMAT0000750, MIMAT0000753, MIMAT0004694, MIMAT0000772, MIMAT0000773, MIMAT0000255, MIMAT0004557, MIMAT0004676, MIMAT0000685, MIMAT0004677, MIMAT0000686, MIMAT0004682, MIMAT0000703, MIMAT0004683, MIMAT0000705, MIMAT0000707, MIMAT0003385, MIMAT0000710, MIMAT0000719, MIMAT0004686, MIMAT0000721, MIMAT0001621, MIMAT0000722, MIMAT0000723, MIMAT0004687, MIMAT0000724, MIMAT0000726, MIMAT0000725, MIMAT0000727, MIMAT0004688, MIMAT0004955, MIMAT0004956, MIMAT0000728, MIMAT0000729, MIMAT0003386, MIMAT0002172, MIMAT0000720, MIMAT0000730, MIMAT0004689, MIMAT0000732, MIMAT0000731, MIMAT0000733, MIMAT0004690, MIMAT0000735, MIMAT0000734, MIMAT0000736, MIMAT0000737, MIMAT0000738, MIMAT0001075, MIMAT0001639, MIMAT0001638, MIMAT0002171, MIMAT0003329, MIMAT0004813, MIMAT0002170, MIMAT0003339, MIMAT0001339, MIMAT0001340, MIMAT0004748, MIMAT0001341, MIMAT0004749, MIMAT0003393, MIMAT0001343, MIMAT0001536, MIMAT0001625, MIMAT0004757, MIMAT0002814, MIMAT0002815, MIMAT0001627, MIMAT0001532, MIMAT0001541, MIMAT0003327, MIMAT0001545, MIMAT0004910, MIMAT0004909, MIMAT0001631, MIMAT0001635, MIMAT0001636, MIMAT0001630, MIMAT0003885, MIMAT0003884, MIMAT0004784, MIMAT0003150, MIMAT0002173, MIMAT0004761, MIMAT0002174, MIMAT0002176, MIMAT0002175, MIMAT0004762, MIMAT0002177, MIMAT0002178, MIMAT0003180, MIMAT0004763, MIMAT0002804, MIMAT0002805, MIMAT0002806, MIMAT0004764, MIMAT0004765, MIMAT0002807, MIMAT0002812, MIMAT0003161, MIMAT0002813, MIMAT0002816, MIMAT0002817, MIMAT0002818, MIMAT0002820, MIMAT0004768, MIMAT0002824, MIMAT0004772, MIMAT0002870, MIMAT0004773, MIMAT0002871, MIMAT0004774, MIMAT0002872, MIMAT0004775, MIMAT0002873, MIMAT0002874, MIMAT0002875, MIMAT0002876, MIMAT0004776, MIMAT0002878, MIMAT0002879, MIMAT0002880, MIMAT0004778, MIMAT0004975, MIMAT0002881, MIMAT0004779, MIMAT0002882, MIMAT0002808, MIMAT0002823, MIMAT0002822, MIMAT0004777, MIMAT0002877, MIMAT0005788, MIMAT0005789, MIMAT0002883, MIMAT0002827, MIMAT0002826, MIMAT0002860, MIMAT0004770, MIMAT0002859, MIMAT0002851, MIMAT0002852, MIMAT0002857, MIMAT0002866, MIMAT0002863, MIMAT0005457, MIMAT0002844, MIMAT0002848, MIMAT0002847, MIMAT0002864, MIMAT0005456, MIMAT0002861, MIMAT0005450, MIMAT0002842, MIMAT0002841, MIMAT0002869, MIMAT0005452, MIMAT0002837, MIMAT0005454, MIMAT0002832, MIMAT0002831, MIMAT0002853, MIMAT0002829, MIMAT0002828, MIMAT0002834, MIMAT0002833, MIMAT0002843, MIMAT0002846, MIMAT0005455, MIMAT0002856, MIMAT0002855, MIMAT0002825, MIMAT0002830, MIMAT0002858, MIMAT0002867, MIMAT0002854, MIMAT0002868, MIMAT0005451, MIMAT0002840, MIMAT0005449, MIMAT0002850, MIMAT0002849, MIMAT0002839, MIMAT0002838, MIMAT0002845, MIMAT0002835, MIMAT0002836, MIMAT0002862, MIMAT0004780, MIMAT0002888, MIMAT0003163, MIMAT0004920, MIMAT0004919, MIMAT0003389, MIMAT0003340, MIMAT0004954, MIMAT0003164, MIMAT0003165, MIMAT0004785, MIMAT0003251, MIMAT0004803, MIMAT0003254, MIMAT0004798, MIMAT0003285, MIMAT0004806, MIMAT0003323, MIMAT0003323, MIMAT0004812, MIMAT0003333, MIMAT0004800, MIMAT0003257, MIMAT0003214, MIMAT0003233, MIMAT0004794, MIMAT0003215, MIMAT0003216, MIMAT0003217, MIMAT0003219, MIMAT0004793, MIMAT0003220, MIMAT0003221, MIMAT0003222, MIMAT0003223, MIMAT0003225, MIMAT0003226, MIMAT0003227, MIMAT0003228, MIMAT0003230, MIMAT0003231, MIMAT0003232, MIMAT0003234, MIMAT0003235, MIMAT0003236, MIMAT0003237, MIMAT0003238, MIMAT0003239, MIMAT0004795, MIMAT0003240, MIMAT0004796, MIMAT0003241, MIMAT0003242, MIMAT0003243, MIMAT0003244, MIMAT0003245, MIMAT0003246, MIMAT0004797, MIMAT0003247, MIMAT0003248, MIMAT0003249, MIMAT0003250, MIMAT0003252, MIMAT0003253, MIMAT0003255, MIMAT0004799, MIMAT0003256, MIMAT0004801, MIMAT0003258, MIMAT0003259, MIMAT0003260, MIMAT0004802, MIMAT0003261, MIMAT0003263, MIMAT0003264, MIMAT0003265, MIMAT0003266, MIMAT0003267, MIMAT0003268, MIMAT0003269, MIMAT0003270, MIMAT0003271, MIMAT0003272, MIMAT0003273, MIMAT0003274, MIMAT0003275, MIMAT0003276, MIMAT0003277, MIMAT0003278, MIMAT0003279, MIMAT0003280, MIMAT0003281, MIMAT0003282, MIMAT0003283, MIMAT0004804, MIMAT0004805, MIMAT0003284, MIMAT0003286, MIMAT0003287, MIMAT0003288, MIMAT0003289, MIMAT0003290, MIMAT0003291, MIMAT0003292, MIMAT0004807, MIMAT0003293, MIMAT0003294, MIMAT0004808, MIMAT0003295, MIMAT0003296, MIMAT0003297, MIMAT0004809, MIMAT0004810, MIMAT0003298, MIMAT0003299, MIMAT0003300, MIMAT0003302, MIMAT0003303, MIMAT0003304, MIMAT0003305, MIMAT0003306, MIMAT0003307, MIMAT0003308, MIMAT0003309, MIMAT0003310, MIMAT0003311, MIMAT0003312, MIMAT0003313, MIMAT0003314, MIMAT0003315, MIMAT0003316, MIMAT0003317, MIMAT0003318, MIMAT0003319, MIMAT0003320, MIMAT0003321, MIMAT0003322, MIMAT0003328, MIMAT0004814, MIMAT0003330, MIMAT0003331, MIMAT0003332, MIMAT0003335, MIMAT0003336, MIMAT0003337, MIMAT0003338, MIMAT0003324, MIMAT0003325, MIMAT0003326, MIMAT0004952, MIMAT0003881, MIMAT0004819, MIMAT0003880, MIMAT0004284, MIMAT0000252, MIMAT0004926, MIMAT0004927, MIMAT0004553, MIMAT0004554, MIMAT0004945, MIMAT0004946, MIMAT0003879, MIMAT0004957, MIMAT0003945, MIMAT0003888, MIMAT0003883, MIMAT0003882, MIMAT0003947, MIMAT0003946, MIMAT0003887, MIMAT0003886, MIMAT0003948, MIMAT0004209, MIMAT0004185, MIMAT0004953, MIMAT0004911, MIMAT0004923, MIMAT0004922, MIMAT0004925, MIMAT0004924, MIMAT0004949, MIMAT0004950, MIMAT0004948, MIMAT0004947, MIMAT0004906, MIMAT0004905, MIMAT0004951, MIMAT0004916, MIMAT0004917, MIMAT0004921, MIMAT0004912, MIMAT0004902, MIMAT0004913, MIMAT0004907, MIMAT0004918, MIMAT0000441, MIMAT0000442, MIMAT0004970, MIMAT0004971, MIMAT0004972, MIMAT0004973, MIMAT0004974, MIMAT0000092, MIMAT0004507, MIMAT0004508, MIMAT0003218, MIMAT0004792, MIMAT0000093, MIMAT0004509, MIMAT0004976, MIMAT0004977, MIMAT0004978, MIMAT0004979, MIMAT0004980, MIMAT0004981, MIMAT0004982, MIMAT0004983, MIMAT0004984, MIMAT0004985, MIMAT0004986, MIMAT0004987, MIMAT0000094, MIMAT0000095, MIMAT0004510, MIMAT0000096, MIMAT0000097, MIMAT0004511, MIMAT0000689, and MIMAT0004678.
In certain embodiments, such an oligomeric compound complementary or identical to a microRNA comprises at least one tetrahydropyran nucleoside of Formula I. In certain embodiments, such oligomeric compound comprises at least two tetrahydropyran nucleosides of Formula I. In certain embodiments, such oligomeric compound complementary or identical to a microRNA comprises at least one tetrahydropyran nucleosides of Formula I and has a motif selected from: gapmer, hemimer, alternating, uniformly modified, and any other motif described herein.

E. pre-mRNA Processing

In certain embodiments, oligomeric compounds provided herein are targeted to a pre-mRNA. In certain embodiments, such oligomeric compounds alter splicing of the pre-mRNA. In certain such embodiments, the ratio of one variant of a target mRNA to another variant of the target mRNA is altered. In certain such embodiments, the ratio of one variant of a target protein to another variant of the target protein is altered. Certain oligomeric compounds and nucleobase sequences that may be used to alter splicing of a pre-mRNA may be found for example in U.S. Pat. No. 6,210,892; U.S. Pat. No. 5,627,274; U.S. Pat. Nos. 5,665,593; 5,916,808; U.S. Pat. No. 5,976,879; US2006/0172962; US2007/002390; US2005/0074801; US2007/0105807; US2005/0054836; WO 2007/090073; WO2007/047913, Hua et al., PLoS Biol 5(4):e73; Vickers et al., J. Immunol. 2006 Mar. 15; 176(6):3652-61, each of which is hereby incorporated by reference in its entirety for any purpose. In certain embodiments antisense sequences that alter splicing are modified according to motifs of the present invention. In certain embodiments, oligomeric compounds of the present invention redirect polyadenylation of pre-mRNA. See, for example Vickers et al., Nucleic Acids Res. 29(6):1293-1299, which is hereby incorporated by reference in its entirety for any purpose. In certain embodiments antisense sequences that redirect polyadenylation are modified according to motifs of the present invention.

In certain embodiments, the invention provides oligomeric compounds complementary to a pre-mRNA encoding Bcl-x. In certain such embodiments, the oligomeric compound alters splicing of Bcl-x. Certain sequences and regions useful for altering splicing of Bcl-x may be found in U.S. Pat. No. 6,172,216; U.S. Pat. No. 6,214,986; U.S. Pat. No. 6,210,892; US2007/002390 and WO 2007/028065, each of which is hereby incorporated by reference in its entirety for any purpose.

In certain embodiments, the present invention provides compounds complementary to a pre-mRNA encoding MyD88. In certain such embodiments, the oligomeric compound alters splicing of MyD88. Certain sequences and regions useful for altering splicing of MyD88 may be found in U.S. application Ser. No. 11/336,785, which is hereby incorporated by reference in its entirety for any purpose.

In certain embodiments, the present invention provides compounds complementary to a pre-mRNA encoding Lamin A (LMN-A). In certain such embodiments, the oligomeric compound alters splicing of Lamin A. Certain sequences and regions useful for altering splicing of Lamin A may be found in PCT/US2006/041018, which is hereby incorporated by reference in its entirety for any purpose.

In certain embodiments, the present invention provides compounds complementary to a pre-mRNA encoding TNF superfamily of receptors. In certain such embodiments, the oligomeric compound alters splicing of TNF. Certain sequences and regions useful for altering splicing of TNF may be found in US2007/0105807, which is hereby incorporated by reference in its entirety for any purpose.

In certain embodiments, the present invention provides compounds complementary to a pre-mRNA encoding SMN2. In certain such embodiments, the oligomeric compound alters splicing of SMN2. Certain sequences and regions useful for altering splicing of SMN2 may be found in PCT/US06/024469, which is hereby incorporated by reference in its entirety for any purpose. In certain embodiments, oligomeric compounds having any motif described herein have a nucleobase sequence complementary to intron 7 of SMN2. Certain such nucleobase sequences are exemplified in the non-limiting table below.

| | | SEQ ID |
|---|---|---|
| Sequence | Length | NO |
| TGCTGGCAGACTTAC | 15 | 9 |
| CATAATGCTGGCAGA | 15 | 10 |
| TCATAATGCTGGCAG | 15 | 11 |
| TTCATAATGCTGGCA | 15 | 12 |
| TTTCATAATGCTGGC | 15 | 13 |
| ATTCACTTTCATAATGCTGG | 20 | 14 |
| TCACTTTCATAATGCTGG | 18 | 15 |
| CTTTCATAATGCTGG | 15 | 16 |
| TCATAATGCTGG | 12 | 17 |
| ACTTTCATAATGCTG | 15 | 18 |
| TTCATAATGCTG | 12 | 19 |
| CACTTTCATAATGCT | 15 | 20 |
| TTTCATAATGCT | 12 | 21 |
| TCACTTTCATAATGC | 15 | 22 |
| CTTTCATAATGC | 12 | 23 |
| TTCACTTTCATAATG | 15 | 24 |
| ACTTTCATAATG | 12 | 25 |
| ATTCACTTTCATAAT | 15 | 26 |
| CACTTTCATAAT | 12 | 27 |
| GATTCACTTTCATAA | 15 | 28 |
| TCACTTTCATAA | 12 | 29 |
| TTCACTTTCATA | 12 | 30 |
| ATTCACTTTCAT | 12 | 31 |
| AGTAAGATTCACTTT | 15 | 32 |

In certain embodiments, such oligomeric compound complementary to a pre-mRNA comprises at least one tetrahydropyran nucleoside of Formula I. In certain embodiments, such oligomeric compound comprises at least two tetrahydropyran nucleosides of Formula I. In certain embodiments, such oligomeric compound complementary to a pre-mRNA comprises at least one tetrahydropyran nucleosides of Formula I and has a motif selected from: gapmer, hemimer, alternating, uniformly modified, and any other motif described herein.

E. Synthesis, Purification and Analysis

Oligomerization of modified and unmodified nucleosides and nucleotides can be routinely performed according to literature procedures for DNA (Protocols for Oligonucleotides and Analogs, Ed. Agrawal (1993), Humana Press) and/or RNA (Scaringe, Methods (2001), 23, 206-217. Gait et al., Applications of Chemically synthesized RNA in RNA: Protein Interactions, Ed. Smith (1998), 1-36. Gallo et al., Tetrahedron (2001), 57, 5707-5713).

Oligomeric compounds provided herein can be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives. The invention is not limited by the method of antisense compound synthesis.

Methods of purification and analysis of oligomeric compounds are known to those skilled in the art. Analysis methods include capillary electrophoresis (CE) and electrospray-mass spectroscopy. Such synthesis and analysis methods can be performed in multi-well plates. The method of the invention is not limited by the method of oligomer purification.

F. Compositions and Methods for Formulating Pharmaceutical Compositions

Oligomeric compounds may be admixed with pharmaceutically acceptable active and/or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

Oligomeric compounds, including antisense compounds, can be utilized in pharmaceutical compositions by combining such oligomeric compounds with a suitable pharmaceutically acceptable diluent or carrier. A pharmaceutically acceptable diluent includes phosphate-buffered saline (PBS). PBS is a diluent suitable for use in compositions to be delivered parenterally. Accordingly, in one embodiment, employed in the methods described herein is a pharmaceutical composition comprising an antisense compound and/or antidote compound and a pharmaceutically acceptable diluent. In certain embodiments, the pharmaceutically acceptable diluent is PBS.

Pharmaceutical compositions comprising oligomeric compounds encompass any pharmaceutically acceptable salts, esters, or salts of such esters. In certain embodiments, pharmaceutical compositions comprising oligomeric compounds comprise one or more oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an oligomeric compound which are cleaved by endogenous nucleases within the body, to form the active oligomeric compound.

Lipid-based vectors have been used in nucleic acid therapies in a variety of methods. In one method, the nucleic acid is introduced into preformed liposomes or lipoplexes made of mixtures of cationic lipids and neutral lipids. In another method, DNA complexes with mono- or poly-cationic lipids are formed without the presence of a neutral lipid.

Certain preparations are described in Akine et al., Nature Biotechnology 26, 561-569 (1 May 2008), which is herein incorporated by reference in its entirety.

### Nonlimiting Disclosure and Incorporation by Reference

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH for the natural 2′-H of DNA) or as an RNA having a modified base (thymine (methylated uracil) for natural uracil of RNA).

Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified bases, such as “AT^{me}CGAUCG,” wherein ^{me}C indicates a cytosine base comprising a methyl group at the 5-position.

Likewise, one of skill will appreciate that in certain circumstances using the conventions described herein, the same compound may be described in more than one way. For example, an antisense oligomeric compound having two non-hybridizing 3′-terminal 2′-MOE modified nucleosides, but otherwise fully complementary to a target nucleic acid may be described as an oligonucleotide comprising a region of 2′-MOE-modified nucleosides, wherein the oligonucleotide is less than 100% complementary to its target. Or that same compound may be described as an oligomeric compound comprising: (1) an oligonucleotide that is 100% complementary to its nucleic acid target and (2) a terminal group wherein the terminal group comprises two 2′-MOE modified terminal-group nucleosides. Such descriptions are not intended to be exclusive of one another or to exclude overlapping subject matter.

### EXAMPLES (GENERAL)

¹H and ¹³C NMR spectra were recorded on a 300 MHz and 75 MHz Bruker spectrometer, respectively.

### Example 1

### Synthesis of Nucleoside Phosphoramidites

The preparation of nucleoside phosphoramidites is performed following procedures that are illustrated herein and in the art such as but not limited to U.S. Pat. No. 6,426,220 and published PCT WO 02/36743.

### Example 2

### Oligonucleoside Synthesis

The oligomeric compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as alkylated derivatives and those having phosphorothioate linkages.

Oligonucleotides: Unsubstituted and substituted phosphodiester (P═O) oligonucleotides can be synthesized on an automated DNA synthesizer (Applied Biosystems model 394) using standard phosphoramidite chemistry with oxidation by iodine.

Phosphorothioates (P═S) are synthesized similar to phosphodiester oligonucleotides with the following exceptions: thiation is effected in certain embodiments by utilizing a 10% w/v solution of 3,H-1,2-benzodithiole-3-one 1,1-dioxide in acetonitrile for the oxidation of the phosphite linkages. The thiation reaction step time is increased to 180 sec and preceded by the normal capping step. After cleavage from the CPG column and deblocking in concentrated ammonium hydroxide at 55° C. (12-16 hr), the oligonucleotides are recovered by precipitating with greater than 3 volumes of ethanol from a 1 M NH₄OAc solution. Phosphinate oligonucleotides can be prepared as described in U.S. Pat. No. 5,508,270.

Alkyl phosphonate oligonucleotides can be prepared as described in U.S. Pat. No. 4,469,863.

3′-Deoxy-3′-methylene phosphonate oligonucleotides can be prepared as described in U.S. Pat. No. 5,610,289 or 5,625,050.

Phosphoramidite oligonucleotides can be prepared as described in U.S. Pat. No. 5,256,775 or U.S. Pat. No. 5,366,878.

Alkylphosphonothioate oligonucleotides can be prepared as described in published PCT applications PCT/US94/00902 and PCT/US93/06976 (published as WO 94/17093 and WO 94/02499, respectively).

3′-Deoxy-3′-amino phosphoramidate oligonucleotides can be prepared as described in U.S. Pat. No. 5,476,925.

Phosphotriester oligonucleotides can be prepared as described in U.S. Pat. No. 5,023,243.

Borano phosphate oligonucleotides can be prepared as described in U.S. Pat. Nos. 5,130,302 and 5,177,198.

Oligonucleosides: Methylenemethylimino linked oligonucleosides, also identified as MMI linked oligonucleosides, methylenedimethylhydrazo linked oligonucleosides, also identified as MDH linked oligonucleosides, and methylenecarbonylamino linked oligonucleosides, also identified as amide-3 linked oligonucleosides, and methyleneaminocarbonyl linked oligonucleosides, also identified as amide-4 linked oligonucleo sides, as well as mixed backbone oligomeric compounds having, for instance, alternating MMI and P═O or P═S linkages can be prepared as described in U.S. Pat. Nos. 5,378,825, 5,386,023, 5,489,677, 5,602,240 and 5,610,289.

Formacetal and thioformacetal linked oligonucleosides can be prepared as described in U.S. Pat. Nos. 5,264,562 and 5,264,564.

Ethylene oxide linked oligonucleosides can be prepared as described in U.S. Pat. No. 5,223,618.

### Example 3

### Oligonucleotide Isolation

After cleavage from the controlled pore glass solid support or other support medium and deblocking in concentrated ammonium hydroxide at 55° C. for 12-16 hours, the oligonucleotides or oligonucleosides are recovered by precipitation out of 1 M NH₄OAc with >3 volumes of ethanol. Synthesized oligonucleotides are analyzed by electrospray mass spectroscopy (molecular weight determination) and by capillary gel electrophoresis. The relative amounts of phosphorothioate and phosphodiester linkages obtained in the synthesis is determined by the ratio of correct molecular weight relative to the −16 amu product (+/−32+/−48). For some studies oligonucleotides are purified by HPLC, as described by Chiang et al., J. Biol. Chem. 1991, 266, 18162-18171. Results obtained with HPLC-purified material are generally similar to those obtained with non-HPLC purified material.

### Example 4

Oligonucleotide Synthesis—96 Well Plate Format

Oligonucleotides can be synthesized via solid phase P(III) phosphoramidite chemistry on an automated synthesizer capable of assembling 96 sequences simultaneously in a 96-well format. Phosphodiester internucleotide linkages are afforded by oxidation with aqueous iodine. Phosphorothioate internucleotide linkages are generated by sulfurization utilizing 3,H-1,2 benzodithiole-3-one 1,1 dioxide (Beaucage Reagent) in anhydrous acetonitrile. Standard base-protected beta-cyanoethyl-diiso-propyl phosphoramidites are purchased from commercial vendors (e.g. PE-Applied Biosystems, Foster City, Calif., or Pharmacia, Piscataway, N.J.). Non-standard nucleosides are synthesized as per standard or patented methods. They are utilized as base protected beta-cyanoethyldiisopropyl phosphoramidites.

Oligonucleotides are cleaved from support and deprotected with concentrated NH₄OH at elevated temperature (55-60° C.) for 12-16 hours and the released product then dried in vacuo. The dried product is then re-suspended in sterile water to afford a master plate from which all analytical and test plate samples are then diluted utilizing robotic pipettors.

### Example 5

### Oligonucleotide Analysis using 96-Well Plate Format

The concentration of oligonucleotide in each well is assessed by dilution of samples and UV absorption spectroscopy. The full-length integrity of the individual products is evaluated by capillary electrophoresis (CE) in either the 96-well format (Beckman P/ACE™ MDQ) or, for individually prepared samples, on a commercial CE apparatus (e.g., Beckman P/ACE™ 5000, ABI 270). Base and backbone composition is confirmed by mass analysis of the oligomeric compounds utilizing electrospray-mass spectroscopy. All assay test plates are diluted from the master plate using single and multi-channel robotic pipettors. Plates are judged to be acceptable if at least 85% of the oligomeric compounds on the plate are at least 85% full length.

### Example 6

### Cell Culture and Oligonucleotide Treatment

The effect of oligomeric compounds on target nucleic acid expression is tested in any of a variety of cell types provided that the target nucleic acid is present at measurable levels. This can be routinely determined using, for example, PCR or Northern blot analysis. Cell lines derived from multiple tissues and species can be obtained from American Type Culture Collection (ATCC, Manassas, Va.).

The following cell type is provided for illustrative purposes, but other cell types can be routinely used, provided that the target is expressed in the cell type chosen. This can be readily determined by methods routine in the art, for example Northern blot analysis, ribonuclease protection assays or RT-PCR.

b.END cells: The mouse brain endothelial cell line b.END was obtained from Dr. Werner Risau at the Max Plank Institute (Bad Nauheim, Germany). b.END cells were routinely cultured in DMEM, high glucose (Invitrogen Life Technologies, Carlsbad, Calif.) supplemented with 10% fetal bovine serum (Invitrogen Life Technologies, Carlsbad, Calif.). Cells were routinely passaged by trypsinization and dilution when they reached approximately 90% confluence. Cells were seeded into 96-well plates (Falcon-Primaria #353872, BD Biosciences, Bedford, Mass.) at a density of approximately 3000 cells/well for uses including but not limited to oligomeric compound transfection experiments.

Experiments involving treatment of cells with oligomeric compounds:

When cells reach appropriate confluency, they are treated with oligomeric compounds using a transfection method as described.

LIPOFECTIN™

When cells reached 65-75% confluency, they are treated with oligonucleotide. Oligonucleotide is mixed with LIPOFECTIN™ Invitrogen Life Technologies, Carlsbad, Calif.) in Opti-MEM™-1 reduced serum medium (Invitrogen Life Technologies, Carlsbad, Calif.) to achieve the desired concentration of oligonucleotide and a LIPOFECTIN™ concentration of 2.5 or 3 μg/mL per 100 nM oligonucleotide. This transfection mixture is incubated at room temperature for approximately 0.5 hours. For cells grown in 96-well plates, wells are washed once with 100 μL OPTI-MEM™-1 and then treated with 130 μL of the transfection mixture. Cells grown in 24-well plates or other standard tissue culture plates are treated similarly, using appropriate volumes of medium and oligonucleotide. Cells are treated and data are obtained in duplicate or triplicate. After approximately 4-7 hours of treatment at 37° C., the medium containing the transfection mixture is replaced with fresh culture medium. Cells are harvested 16-24 hours after oligonucleotide treatment.

Other suitable transfection reagents known in the art include, but are not limited to, CYTOFECTIN™, LIPOFECTAMINE™, OLIGOFECTAMINE™, and FUGENE™. Other suitable transfection methods known in the art include, but are not limited to, electroporation.

### Example 7

### Real-Time Quantitative PCR Analysis of Target mRNA Levels

Quantitation of a target mRNA levels was accomplished by real-time quantitative PCR using the ABI PRISM™ 7600, 7700, or 7900 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR) products in real-time. As opposed to standard PCR in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., FAM or JOE, obtained from either PE-Applied Biosystems, Foster City, Calif., Operon Technologies Inc., Alameda, Calif. or Integrated DNA Technologies Inc., Coralville, Iowa) is attached to the 5′ end of the probe and a quencher dye (e.g., TAMRA, obtained from either PE-Applied Biosystems, Foster City, Calif., Operon Technologies Inc., Alameda, Calif. or Integrated DNA Technologies Inc., Coralville, Iowa) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular intervals by laser optics built into the ABI PRISM™ Sequence Detection System. In each assay, a series of parallel reactions containing serial dilutions of mRNA from untreated control samples generates a standard curve that is used to quantitate the percent inhibition after antisense oligonucleotide treatment of test samples.

Prior to quantitative PCR analysis, primer-probe sets specific to the target gene being measured are evaluated for their ability to be “multiplexed” with a GAPDH amplification reaction. In multiplexing, both the target gene and the internal standard gene GAPDH are amplified concurrently in a single sample. In this analysis, mRNA isolated from untreated cells is serially diluted. Each dilution is amplified in the presence of primer-probe sets specific for GAPDH only, target gene only (“single-plexing”), or both (multiplexing). Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed multiplexable. Other methods of PCR are also known in the art.

RT and PCR reagents were obtained from Invitrogen Life Technologies (Carlsbad, Calif.). RT, real-time PCR was carried out by adding 20 μL PCR cocktail (2.5×PCR buffer minus MgCl₂, 6.6 mM MgCl₂, 375 μM each of dATP, dCTP, dCTP and dGTP, 375 nM each of forward primer and reverse primer, 125 nM of probe, 4 Units RNAse inhibitor, 1.25 Units PLATINUM® Taq, 5 Units MuLV reverse transcriptase, and 2.5×ROX dye) to 96-well plates containing 30 μL total RNA solution (20-200 ng). The RT reaction was carried out by incubation for 30 minutes at 48° C. Following a 10 minute incubation at 95° C. to activate the PLATINUM® Taq, 40 cycles of a two-step PCR protocol were carried out: 95° C. for 15 seconds (denaturation) followed by 60° C. for 1.5 minutes (annealing/extension).

Gene target quantities obtained by RT, real-time PCR are normalized using either the expression level of GAPDH, a gene whose expression is constant, or by quantifying total RNA using RIBOGREEN™ (Molecular Probes, Inc. Eugene, Oreg.). GAPDH expression is quantified by real time RT-PCR, by being run simultaneously with the target, multiplexing, or separately. Total RNA is quantified using RiboGreen™ RNA quantification reagent (Molecular Probes, Inc. Eugene, Oreg.). Methods of RNA quantification by RIBOGREEN™ are taught in Jones, L. J., et al, (Analytical Biochemistry, 1998, 265, 368-374).

In this assay, 170 μL of RIBOGREEN™ working reagent (RIBOGREEN™ reagent diluted 1:350 in 10 mM Tris-HCl, 1 mM EDTA, pH 7.5) is pipetted into a 96-well plate containing 30 μL purified, cellular RNA. The plate is read in a CytoFluor 4000 (PE Applied Biosystems) with excitation at 485 nm and emission at 530 nm.

### Example 8

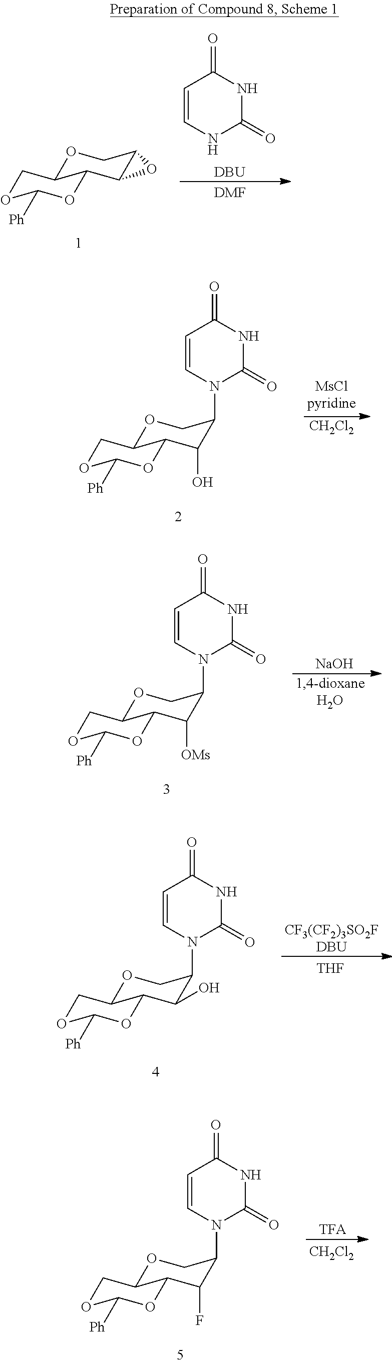

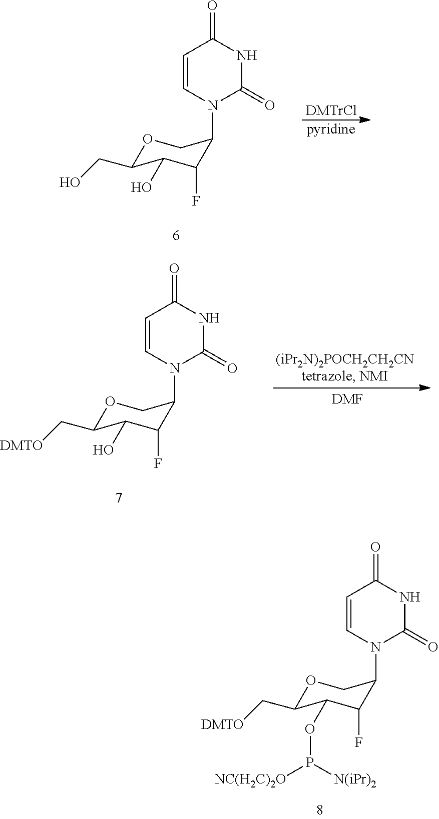

### a) Preparation of Compound 2

Compound 1 (13.1 g, 55.9 mmol, 1,5:2,3-dianhydro-4,6-O-benzylidene-D-allitol, purchased from Carbosynth, UK), was dissolved in anhydrous N,N-dimethylformamide (210 mL). To this solution was added uracil (7.52 g, 67.1 mmol) and 1,8-diazabicyclo[5.4.0]undec-7-ene (10.0 mL, 67.1 mmol). This mixture was heated to 85° C. for 7 hours. The mixture was then cooled to room temperature, poured into ethyl acetate (1 L), and washed with half-saturated aqueous NaHCO₃ (4×1 L). The aqueous portion was dried over anhydrous Na₂SO₄, filtered, and evaporated to a pale foam, which was purified by silica gel chromatography (2% methanol in CH₂Cl₂) to yield 12.5 g (64.5% yield) of Compound 2 as a white foam. ESI-MS [M+H⁺]: calc. 347 Da; obs. 347 Da. ¹H NMR was consistent with structure. Reference for this procedure—Abramov, M.; Marchand, A.; Calleja-Marchand, A.; Herdewijn, P. Synthesis of D-Altritol Nucleosides with a 3′-O-tert-butyldimethylsilyl protecting group. Nucleosides, Nucleotides & Nucleic Acids (2004) 23, 439.

### b) Preparation of Compound 3

Compound 2 (12.1 g, 35.0 mmol) was dissolved in a mixture of anhydrous CH₂Cl₂ (50 mL) and anhydrous pyridine (50 mL). This mixture was cooled to 0° C., then treated with methane-sulfonyl chloride (6.77 mL, 87.4 mmol). After maintaining at 0° C. for 15 minutes, the mixture was warmed to room temperature and stirred an additional 5 hours. Concentration in vacuo yielded a golden slush, which was resuspended in CH₂Cl₂ (500 mL), washed with half-saturated aq. NaHCO₃, dried over anhydrous Na₂SO₄, filtered, and evaporated to a golden oil. Subsequent purification by silica gel chromatography (2% methanol in CH₂Cl₂) yielded 11.7 g (78.6% yield) of Compound 3 as a pale yellow foam. ESI-MS [M+H⁺]: calc. 425 Da; obs. 425 Da. ¹H NMR was consistent with structure.

### c) Preparation of Compound 4

Compound 3 (11.2 g, 26.5 mmol) was suspended in 1,4-dioxane (100 mL). To this suspension was added 100 mL of 2M aqueous NaOH. The resulting mixture was warmed to 60° C. and stirred for 3.5 hours. The mixture was cooled to room temperature, then neutralized with acetic acid (11.4 mL). The mixture was concentrated in vacuo to ˜100 mL and then poured into CH₂Cl₂ (500 mL). The resulting mixture was washed with saturated aq. NaHCO₃ (500 mL), dried over anhydrous Na₂SO₄, filtered, and evaporated to yield 8.23 g (89.7% yield) of Compound 4 as an off-white solid. ESI-MS [M+H⁺]: calc. 347 Da; obs. 347 Da. ¹H NMR was consistent with structure.

### d) Preparation of Compound 5

Compound 4 (7.96 g, 23.0 mmol) was dissolved in anhydrous THF (100 mL). To this solution was added 1,8-diazabicyclo[5.4.0]undec-7-ene (5.1 mL, 34 mmol), followed by nonafluorobutanesulfonyl fluoride (11.6 mL, 34 mmol), which was added dropwise with stirring. This mixture was incubated at 30° C. for 84 hours. The mixture was poured into ethyl acetate (400 mL), washed with half-saturated aq. NaHCO₃ (2×500 mL), dried over anhydrous Na₂SO₄, filtered, and evaporated to a pale foam. Silica gel chromatography (1:1 hexanes:ethyl acetate) yielded 7.92 g of Compound 5 as an impure mixture. This mixture was used for subsequent reactions without further purification. A small portion was more carefully purified by silica gel chromatography for analytical characterization. ESI-MS [M+H⁺]: calc. 349 Da; obs. 349 Da (major impurity [M+H⁺]=329, consistent with elimination of HF). Both ¹H and ¹⁹F NMR were consistent with structure for Compound 5.

### e) Preparation of Compound 6

Impure Compound 5 (6.87 g, 19.7 mmol) was dissolved in anhydrous CH₂Cl₂ (100 mL). To this solution was added trifluoroacetic acid (35 mL). After stirring at room temperature for 1 hour, this mixture was concentrated in vacuo to a pale-orange oil. Purification by silica gel chromatography (stepwise gradient from 1% methanol to 10% methanol in CH₂Cl₂) yielded 3.58 g (69% yield) of Compound 6 as a white foam. ESI-MS [M+H⁺]: calc. 261 Da; obs. 261 Da.

### f) Preparation of Compound 7

Compound 6 (3.37 g, 12.9 mmol) was dissolved in anhydrous pyridine (40 mL). After cooling to 0° C., the solution was treated with 4,4′-dimethoxytrityl chloride (6.59 g, 19.5 mmol). After stirring at 0° C. for 20 minutes, the mixture was warmed to room temperature for an additional 3 hours. The resulting mixture was concentrated in vacuo to a brown oil, resuspended in CH₂Cl₂ (400 mL), washed with half-saturated aq. NaHCO₃ (2×400 mL), dried over anhydrous Na₂SO₄, filtered, and evaporated. Silica gel chromatography (2% v/v methanol in CH₂Cl₂, yielded 5.68 g (77.9% yield) of Compound 7 as a beige foam. Both ¹H and ¹⁹F NMR were consistent with structure.

### g) Preparation of Compound 8

Compound 7 (2.50 g, 4.45 mmol) was dissolved in anhydrous N,N-dimethylformamide (11.2 mL). To this solution was added 2-cyanoethyl-N,N,N′,N′-tetraisopropylphosphorodiamidite (1.98 mL, 6.23 mmol), tetrazole (156 mg, 2.22 mmol), and N-methylimidazole (89 μL, 1.11 mmol). After stirring at room temperature for 3 hours, the mixture was treated with triethylamine (2.48 mL, 17.8 mmol), stirred for 5 minutes, then poured into ethyl acetate (250 mL). The resulting solution was washed with 1:1 saturated aq. NaHCO₃:saturated aq. NaCl (1×200 mL), followed by saturated aq. NaCl (1×200 mL). The organic portion was dried over anhydrous Na₂SO₄, filtered, and evaporated. Silica gel chromatography (1:1 hexanes:ethyl acetate) yielded 2.61 g (76.8% yield) of Compound 8 as a pale yellow foam. ¹H, ¹⁹F, and ³¹P NMR were consistent with the structure of Compound 8 as a mixture of phosphorous diastereomers.

### Example 9

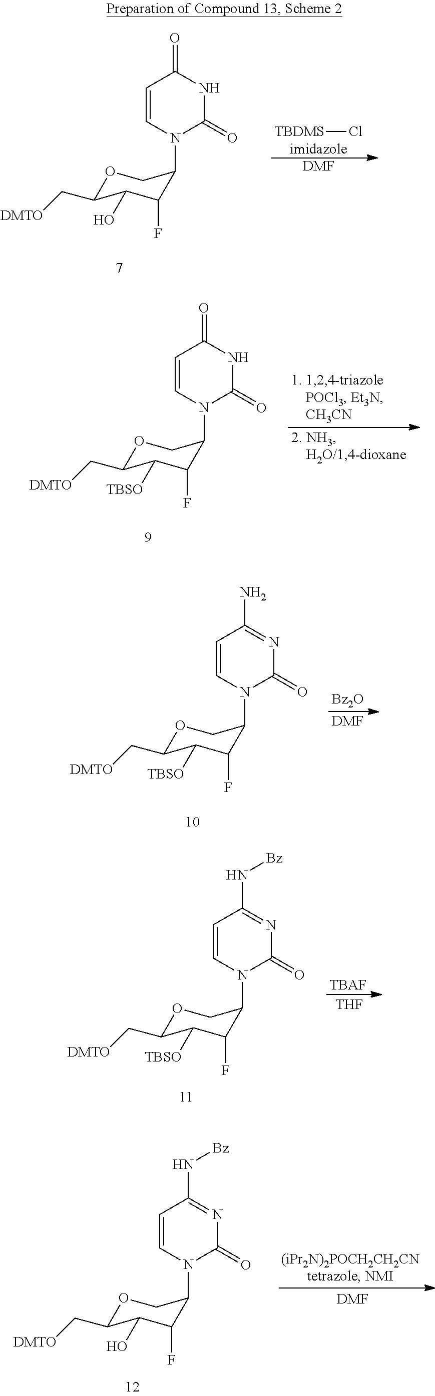

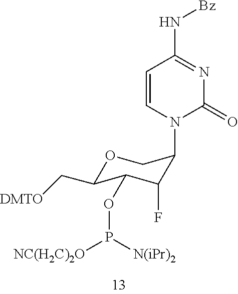

### a) Preparation of Compound 9

Compound 7 (2.50 g, 4.44 mmol, prepared in the previous example) was dissolved in anhydrous N,N-dimethylformamide (10 mL). To this solution was added imidazole (1.82 g, 26.7 mmol) and tert-butyldimethylsilyl chloride (1.34 g, 8.88 mmol). After stirring at room temperature for 12 hours, the mixture was poured into ethyl acetate (250 mL), washed with half-saturated aq. NaHCO₃ (2×200 mL) and saturated aq. NaCl (2×200 mL), dried over anhydrous Na₂SO₄, filtered, and evaporated. Silica gel chromatography (1:1 hexanes:ethyl acetate) yielded 2.52 g (83.8% yield) of Compound 9 as a white foam. ¹H and ¹⁹F NMR were consistent with the indicated structure.

### b) Preparation of Compound 10

To a chilled (0° C.) suspension of 1,2,4-triazole (3.40 g, 49.2 mmol) in anhydrous acetonitrile (44 mL) was added phosphorous oxychloride (1.31 mL, 14.1 mmol). After stirring at 0° C. for 20 minutes, triethylamine (9.8 mL, 70 mmol) was added to the mixture. To the resulting slurry was added a solution of Compound 9 (2.38 g, 3.52 mmol) in anhydrous acetonitrile (20 mL). The mixture was held at 0° C. for 1 hour, then warmed to room temperature for 2 hours. The mixture was subsequently concentrated to approximately half its original volume, poured into ethyl acetate (250 mL), washed with half-saturated aq. NaCl (2×200 mL), dried over anhydrous Na₂SO₄, filtered, and evaporated to a yellow foam. This residue was redissolved in 1,4-dioxane (20 mL) and treated with conc. aq. NH₄OH (20 mL). The reaction vessel was sealed and stirred at room temperature for 12 hours, at which time the mixture was concentrated under reduced pressure, poured into CH₂Cl₂ (200 mL), washed with half-saturated aq. NaHCO₃ (1×200 mL), dried over anhydrous Na₂SO₄, filtered, and evaporated. Silica gel chromatography (1.5% v/v methanol in CH₂Cl₂) yielded 1.98 g (83.4%) of Compound 10 as a yellow foam. ESI-MS [M−H⁺]: calc. 674.8 Da; obs. 674.3 Da. ¹H and ¹⁹F NMR were consistent with structure.

### c) Preparation of Compound 11

Compound 10 (1.86 g, 2.76 mmol) was dissolved in anhydrous N,N-dimethylformamide (10 mL). To the resulting solution was added benzoic anhydride (938 mg, 4.14 mmol). After stirring at room temperature for 14 hours, the mixture was poured into ethyl acetate (250 mL), washed with saturated aq. NaHCO₃ (1×200 mL) and half-saturated aq. NaCl (2×200 mL), dried over anhydrous Na₂SO₄, filtered and evaporated. Silica gel chromatography (1:1 hexanes:ethyl acetate) yielded 2.12 g (98.4%) of Compound 11 as a white foam. ESI-MS [M−H⁺]: calc. 778 Da; obs. 778 Da. ¹H and ¹⁹F NMR were consistent with structure.

### d) Preparation of Compound 12

Compound 11 (1.98 g, 2.54 mmol) was dissolved in anhydrous THF (3 mL). To this solution was added 3.3 mL of 1 M tetrabutylammonium fluoride in THF. After 13 hours, the mixture was evaporated, redissolved in CH₂Cl₂, and subjected to silica gel chromatography. Elution with 1.5% (v/v) methanol in CH₂Cl₂ yielded 1.58 g (93.9%) of Compound 12 as an off-white foam. ESI-MS [M−H⁺]: calc. 664.7 Da; obs. 664.2 Da. ¹H and ¹⁹F NMR were consistent with structure.

### e) Preparation of Compound 13

Compound 12 (1.52 g, 2.28 mmol) was dissolved in anhydrous N,N-dimethylformamide (5.8 mL). To this solution was added 2-cyanoethyl-N,N,N′,N′-tetraisopropylphosphorodiamidite (1.00 mL, 3.19 mmol), tetrazole (80 mg, 1.14 mmol), and N-methylimidazole (45 μL, 0.57 mmol). After stirring at room temperature for 3 hours, the mixture was treated with triethylamine (1.27 mL, 9.13 mmol), stirred for 5 minutes, and then poured into ethyl acetate (200 mL). The resulting solution was washed with 1:1 saturated aq. NaHCO₃:saturated aq. NaCl (1×200 mL), followed by saturated aq. NaCl (2×200 mL). The organic portion was dried over anhydrous Na₂SO₄, filtered, and evaporated. Silica gel chromatography (1:1 hexanes:ethyl acetate) yielded 1.58 g (80.1% yield) of Compound 13 as a pale yellow foam. ¹H, ¹⁹F, and ³¹P NMR were consistent with the structure of Compound 13 as a mixture of phosphorous diastereomers.

### Example 10

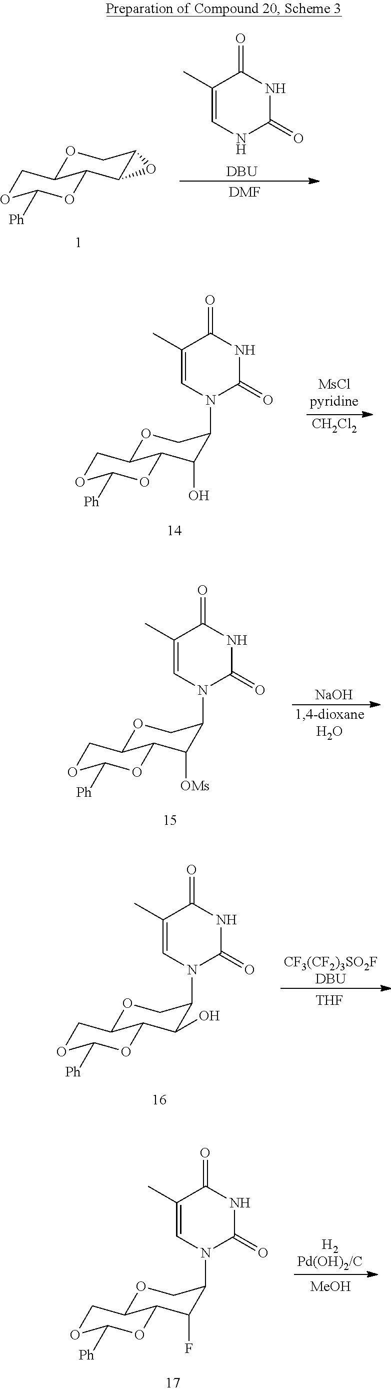

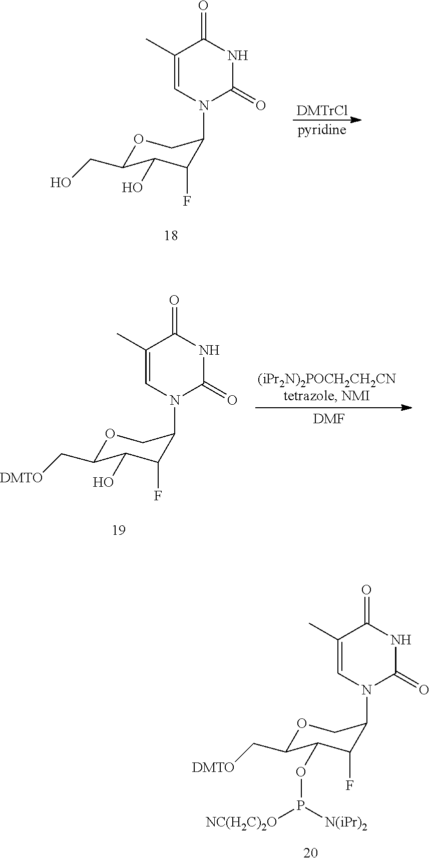

### a) Preparation of Compound 14

Compound 1 (30.0 g, 128 mmol), was dissolved in anhydrous acetonitrile (600 mL). To this solution was added thymine (48.4 g, 384 mmol) and 1,8-diazabicyclo[5.4.0]undec-7-ene (57.4 mL, 384 mmol). This mixture was heated to 85° C. for 12 hours. After cooling to room temperature, unreacted thymine was removed by filtration. The filtered solution was concentrated in vacuo to a yellow oil, redissolved in CH₂Cl₂ (500 mL), washed with saturated aqueous NaHCO₃ (2×500 mL), dried over Na₂SO₄, filtered, and concentrated to a yellow oil. Silica gel chromatography (2% methanol in CH₂Cl₂) of the dried residue yielded 30.3 g (65.6%) of Compound 14 as an off-white foam. ¹H NMR was consistent with structure. ESI-MS [M+H⁺]: calc. 361.4 Da; obs. 361.1 Da.

### b) Preparation of Compound 15

Compound 14 (30.1 g, 83.6 mmol) was dissolved in a mixture of anhydrous CH₂Cl₂ (100 mL) and anhydrous pyridine (100 mL). This mixture was cooled to 0° C., then treated with methane-sulfonyl chloride (8.4 mL, 109 mmol). The mixture was kept at 0° C. for 30 minutes, then warmed to room temperature and stirred for an additional 24 hours. The mixture was concentrated in vacuo to an orange oil, which was redissolved in CH₂Cl₂ (500 mL), washed with half-saturated aq. NaHCO₃ (2×500 mL), dried over anhydrous Na₂SO₄, filtered, and evaporated to a pale orange foam. ¹H NMR was consistent with structure. ESI-MS [M+H⁺]: calc. 439.4 Da; obs. 439.1 Da. The resulting material was used for subsequent reaction without any additional purification.

### c) Preparation of Compound 16

Compound 15 (approximately 34 g crude, 78 mmol) was suspended in 1,4-dioxane (125 mL). To this suspension was added 125 mL of 2M aqueous NaOH. The resulting mixture was warmed to 60° C. and stirred for 3 hours. The mixture was cooled to room temperature, then neutralized with acetic acid (14 mL). The mixture was concentrated in vacuo to ˜75 mL, then poured into CH₂Cl₂ (1.75 L). The mixture was washed with saturated aq. NaHCO₃ (2×1.5 L), dried over anhydrous Na₂SO₄, filtered and evaporated to yield a yellow solid, which was used for subsequent reaction without any additional purification. ESI-MS [M+H⁺]: calc. 361.4 Da; obs. 361.1 Da. ¹H NMR was consistent with structure.

### d) Preparation of Compound 17

Compound 16 (26.6 g crude, 73.8 mmol) was dissolved in anhydrous THF (450 mL). To this solution was added 1,8-diazabicyclo[5.4.0]undec-7-ene (16.5 mL, 111 mmol), followed by nonafluorobutanesulfonyl fluoride (34 mL, 111 mmol), which was added dropwise with stirring. This mixture was incubated at 30° C. for 42 hours. The resulting mixture was concentrated to ˜75 mL, then poured into EtOAc (500 mL), washed with half-saturated aq. NaHCO₃ (2×500 mL), dried over anhydrous Na₂SO₄, filtered and evaporated to a brown oil. Silica gel chromatography (3:2 hexanes:ethyl acetate) yielded 18.1 g (67.8%) of Compound 17 as an impure mixture (˜82% pure by both LCMS and ¹H NMR). This mixture was used for subsequent reactions without further purification. ESI-MS [M+H⁺]: calc. 363 Da; obs. 363 Da (major impurity [M+H⁺]=343, consistent with elimination of HF).

### e) Preparation of Compound 18

Impure Compound 17 (4.57 g, 12.6 mmol) was dissolved in methanol (300 mL). To this solution was added Pd(OH)₂/C (9 g). Flask was flushed with H₂ gas, sealed, and maintained with an H₂ atmosphere while stirring at room temperature. After 12 hours the H₂ gas was vented, Pd(OH)₂/C was removed by filtration through a celite plug, which was washed thoroughly with additional methanol. Concentrated in vacuo to a white foam. Silica gel chromatography (5% methanol in CH₂Cl₂), yielded 10.7 g (95%) of 18 as a white foam. ESI-MS [M+H⁺]: calc. 275.2 Da; obs. 275.1 Da. Both ¹H NMR and ¹⁹F NMR were consistent with structure.

### f) Preparation of Compound 19

Compound 18 (10.6 g, 38.6 mmol) was dissolved in anhydrous pyridine (120 mL), cooled to 0° C. and treated with 4,4′-dimethoxytrityl chloride (26.1 g, 77.2 mmol). The resulting solution was slowly warmed to room temperature and stirred for 14 hours. The reaction mixture was quenched with methanol (10 mL) and concentrated in vacuo to a brown slush. The mixture was redissolved in CH₂Cl₂ (500 mL), washed with half-saturated aqueous NaHCO₃ (2×500 mL), dried over anhydrous Na₂SO₄, filtered and evaporated to a sticky brown foam. Silica gel chromatography (1% methanol in CH₂Cl₂) yielded 20.3 g (91%) of Compound 19 as a yellow foam. ¹H NMR was consistent with structure.

### g) Preparation of Compound 20

Compound 19 (9.00 g, 15.6 mmol) was dissolved in anhydrous N,N-dimethylformamide (37 mL) and 2-cyanoethyl-N,N,N′,N′-tetraisopropylphosphorodiamidite (7.43 mL, 23.4 mmol), tetrazole (656 mg, 9.37 mmol), and N-methylimidazole (311 μL, 3.9 mmol) were added. After stirring at room temperature for 3 hours, the mixture was treated with triethylamine (8.7 mL, 62.4 mmol), stirred for 5 minutes, then poured into ethyl acetate (500 mL). The resulting solution was washed with half-saturated aqueous NaHCO₃ (3×500 mL), dried over anhydrous Na₂SO₄, filtered and evaporated to a sticky yellow foam. Silica gel chromatography (2:3 hexanes:ethyl acetate), followed by precipitation from hexanes/ethyl acetate yielded 10.5 g (87% yield) of Compound 20 as a pale yellow foam. ¹H, ¹⁹F, and ³¹P NMR were consistent with the structure as a mixture of diastereomers.

### Example 11

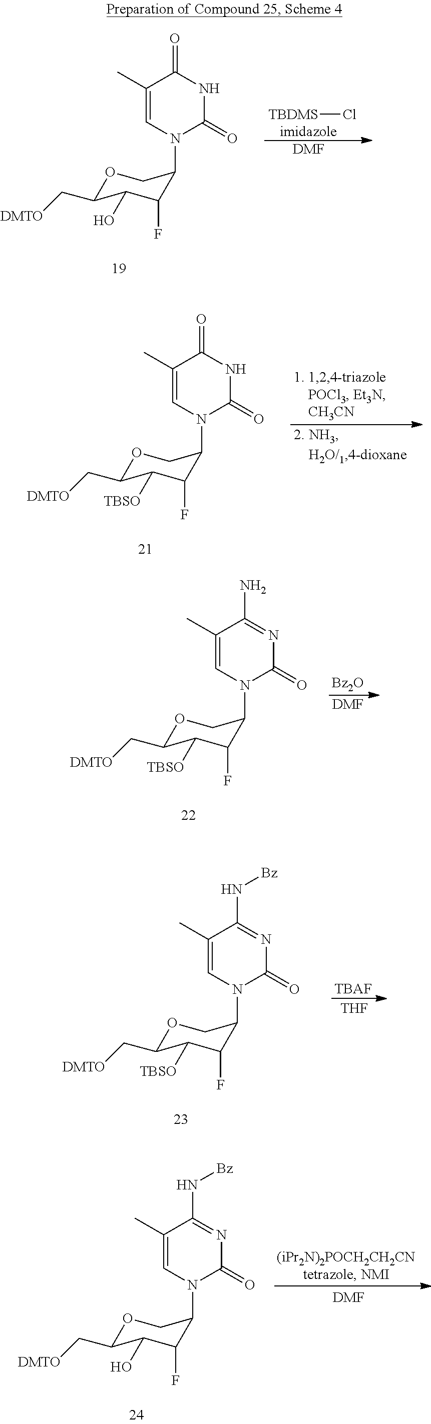

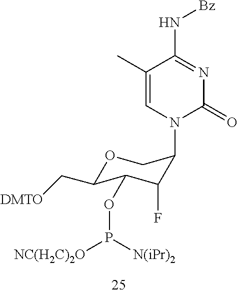

### a) Preparation of Compound 21

Compound 19 (11.2 g, 19.4 mmol, prepared in the previous example) was dissolved in anhydrous N,N-dimethylformamide (44 mL). To this solution was added imidazole (7.9 g, 116 mmol) and tert-butyldimethylsilyl chloride (5.85 g, 38.8 mmol). After stirring at room temperature for 14 hours, quenched with the addition of methanol (10 mL), poured into ethyl acetate (500 mL), washed with half-saturated aq. NaHCO₃ (3×500 mL), dried over anhydrous Na₂SO₄, filtered, and evaporated to 13.2 g (98%) of Compound 21 as a pale yellow foam. ¹H NMR was consistent with the indicated structure. Material was used for subsequent reaction without additional purification.

### b) Preparation of Compound 22

To a chilled (0° C.) suspension of 1,2,4-triazole (18.4 g, 267 mmol) in anhydrous acetonitrile (350 mL) was added phosphorous oxychloride (7.1 mL, 76 mmol). After stirring at 0° C. for 30 minutes, triethylamine (53 mL, 382 mmol) was added to the mixture. To the resulting slurry was added a solution of Compound 21 (13.2 g, 19.1 mmol) in anhydrous acetonitrile (100 mL). The mixture was held at 0° C. for 1 hour, then warmed to room temperature for 3.5 hours. The mixture was subsequently concentrated to approximately half its original volume, poured into ethyl acetate (500 mL), washed with half-saturated aq. NaCl (2×500 mL), dried over anhydrous Na₂SO₄, filtered, and evaporated to a yellow foam. This residue was redissolved in 1,4-dioxane (175 mL) and treated with conc. aq. NH₄OH (175 mL). The reaction vessel was sealed and stirred at room temperature for 14 hours, at which time the mixture was concentrated under reduced pressure, poured into CH₂Cl₂ (500 mL), washed with half-saturated aq. NaHCO₃ (2×500 mL), dried over anhydrous Na₂SO₄, filtered, and evaporated to 12.4 g (94%) of Compound 22 as a yellow foam, which crystallized upon drying overnight. ¹H NMR was consistent with structure. Material was used for subsequent reaction without additional purification.

### c) Preparation of Compound 23

Compound 22 (12.3 g, 17.8 mmol) was dissolved in anhydrous N,N-dimethylformamide (60 mL). To the resulting solution was added benzoic anhydride (6.05 g, 26.7 mmol). After stirring at room temperature for 12 hours, the mixture was poured into ethyl acetate (500 mL), washed with half-saturated aq. NaHCO₃ (3×500 mL), dried over anhydrous Na₂SO₄, filtered and evaporated. Silica gel chromatography (3:1 hexanes:ethyl acetate) yielded 13.4 g (95.1%) of Compound 23 as a white foam. ¹H NMR was consistent with structure.

### d) Preparation of Compound 24

Compound 23 (13.4 g, 16.9 mmol) was dissolved in anhydrous THF (14 mL). To this solution was added 22 mL of 1 M tetrabutylammonium fluoride in THF. After 5 hours, the mixture was evaporated, then subjected to silica gel chromatography. Elution with 2:1 hexanes:ethyl acetate yielded 9.57 g (83.2%) of Compound 24 as a white foam. ¹H NMR was consistent with structure.

### e) Preparation of Compound 25

Compound 24 (9.5 g, 14.0 mmol) was dissolved in anhydrous N,N-dimethylformamide (33 mL). To this solution was added 2-cyanoethyl-N,N,N′,N′-tetraisopropylphosphorodiamidite (6.7 mL, 21.0 mmol), tetrazole (589 mg, 8.41 mmol), and N-methylimidazole (279 μL, 3.50 mmol). After stirring at room temperature for 3 hours, the mixture was treated with triethylamine (7.8 mL, 56 mmol), stirred for 5 minutes, then poured into ethyl acetate (500 mL). The resulting solution was washed with saturated aq. NaCl (3×500 mL), dried over anhydrous Na₂SO₄, filtered, and evaporated. Silica gel chromatography (3:1 hexanes:ethyl acetate) yielded 11.8 g (95% yield) of Compound 25 as a white foam. ¹H and ³¹P NMR were consistent with the structure of Compound 25 as a mixture of phosphorous diastereomers.

### Example 12

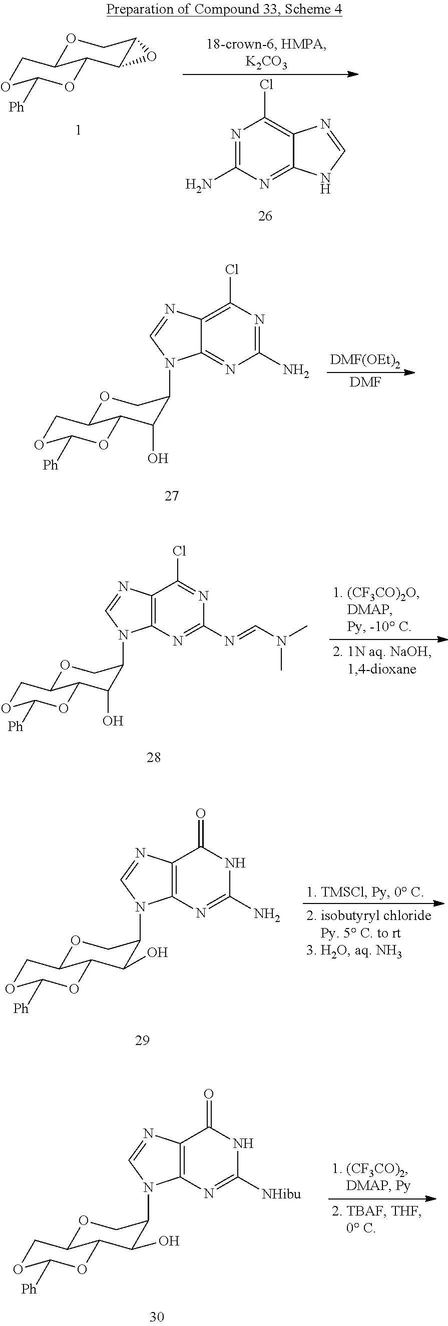

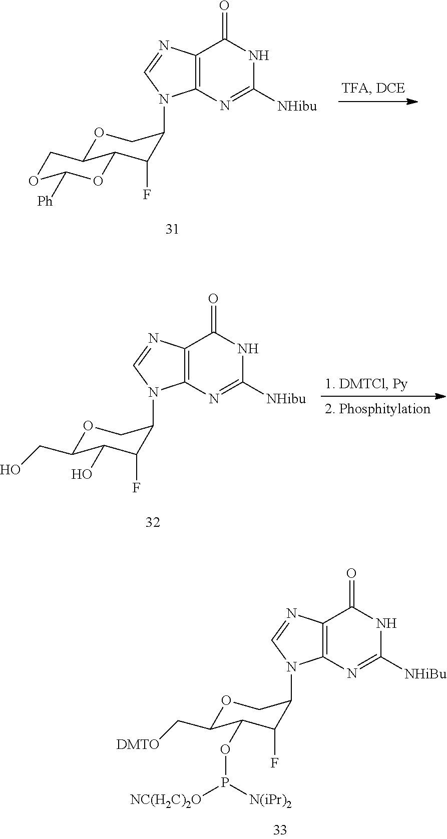

### a) Preparation of Compound 27

Compound 1 (5.40 g, 4.56 mmol, 1,5:2,3-dianhydro-4,6-O-benzylidene-D-allitol, purchased from Carbosynth, UK) was mixed with 2-amino-6-chloropurine Compound 26 (5.89 g, 34.69 mmol) and dried over P₂O₅ under reduced pressure overnight. The mixture was suspended in anhydrous hexamethyl phosphoramide (86 mL) and 18-crown-6 (2.86 g, 10.82 mmol) and K₂CO₃ (3.46 g, 25.04 mmol) was added. The reaction mixture was stirred at 90° C. for 3 hours and allowed to equilibrate to room temperature. Crushed ice was added with subsequent stirring for 1 hour. The precipitate formed was filtered and washed with cold water followed by diethyl ether. The crude material was purified by silica gel column chromatography eluting with 5% MeOH in CH₂Cl₂ to yield Compound 27 (7.01 g, 75%). ¹H NMR (300 MHz, DMSO-d₆) δ 3.61 (m, 1H), 3.78 (t, J=10.1 Hz, 1H), 3.92 (m, 1H), 4.18-4.28 (m, 4H), 5.63 (1, 1H), 5.83 (d, J=4.2 Hz, 1H), 5.40 (d, J=6.3 Hz, 1H), 5.85 (d, J=3.8 Hz, 1H), 6.99 (s, 2H), 7.31-7.42 (m, 5H), 8.21 (s, 1H); MS (ES) m/z 404.0 [M+H]⁻.

### Example 13

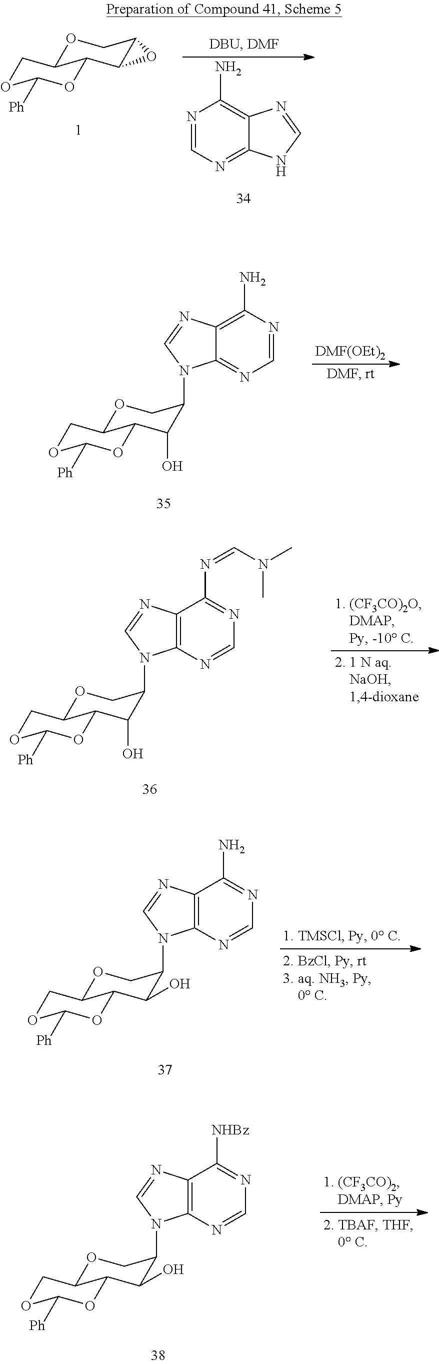

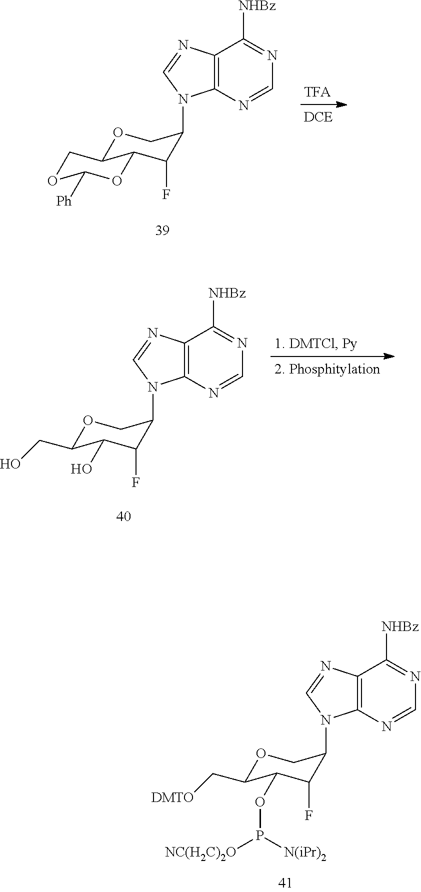

Compound 1, 1, 5:2, 3-dianhydro-4,6-O-benzylidene-D-allitol, is purchased from Carbosynth, UK.

### Example 14

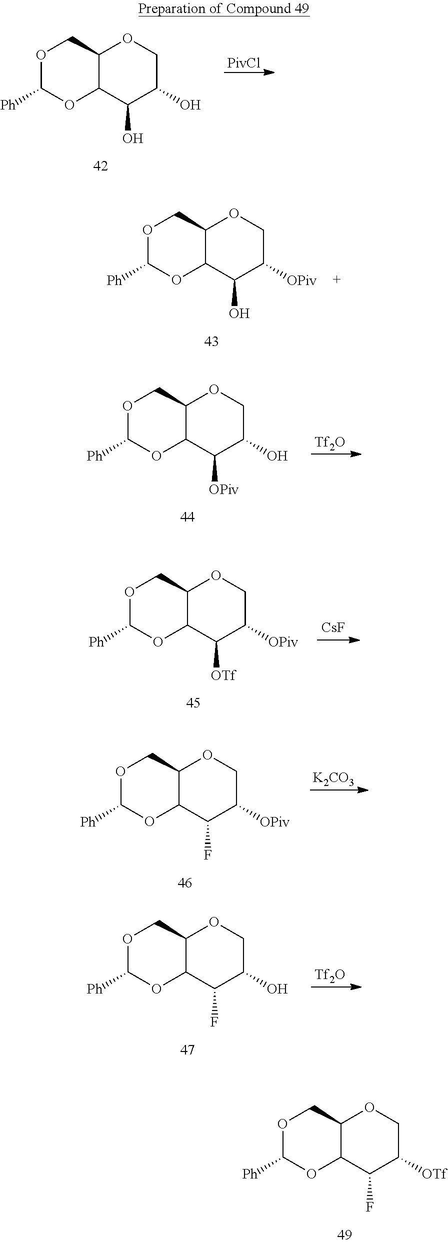

### a) Preparation of Compound 43

Pivaloyl chloride (5.5 mmol, 0.67 mL) was added to a solution of commercially available 1,5-anhydro-4,6-O-benzylidene-D-glucitol (Carbosynth Limited, UK.) Compound 42 (5 mmol, 1.25 g), triethylamine (5.5 mmol, 0.77 mL) and dimethylaminopyridine (20 mg) in dichloromethane (25 mL). After stirring at room temperature for 24 hours, the reaction was diluted with dichloromethane and washed with 5% HCl, saturated sodium bicarbonate and brine then dried (Na2SO4) and concentrated. Purification by column chromatography (silica gel, eluting with 10 to 30% ethyl acetate in hexanes) provided Compound 43 (1.06 g) and Compound 44 (0.64 g) as white solids. Compound 43: ¹H NMR (300 MHz, chloroform-d) δ=7.56-7.44 (m, 2H), 7.36 (m, 3H), 5.49 (s, 1 H), 4.98-4.81 (m, 1H), 4.40-4.22 (m, 1H), 4.16-3.99 (m, 1H), 3.82 (s, 1H), 3.65 (s, 1H), 3.46 (s, 1H), 3.41-3.27 (m, 1H), 3.27-3.15 (m, 1H), 3.04-2.80 (m, 1H), 1.29-1.16 (m, 9H). Compound 44: ¹H NMR (300 MHz, chloroform-d) δ=7.49-7.40 (m, 2H), 7.39-7.32 (m, 3H), 5.53 (s, 1H), 5.08-4.91 (m, 1H), 4.42-4.29 (m, 1H), 4.19-4.04 (m, 1H), 3.92-3.76 (m, 1H), 3.76-3.55 (m, 2H), 3.50-3.30 (m, 2H), 1.24 (s, 9H).

### b) Preparation of Compound 46

Trifluoromethanesulfonic anhydride (4.8 mmol, 0.8 mL) was added to a cold (0° C.) solution of Compound 43 (3.2 mmol, 1.07 g) and pyridine (0.5 mL). After stirring for one hour the reaction was quenched by adding water and the organic layer was washed with water and brine then dried (Na₂SO₄) and concentrated to provide crude Compound 45 which was used without any further purification. ¹H NMR (300 MHz, chloroform-d) δ=7.53-7.42 (m, 2H), 7.42-7.32 (m, 3H), 5.59 (s, 1H), 5.10 (s, 2H), 4.48-4.33 (m, 1H), 4.32-4.15 (m, 1H), 3.90-3.69 (m, 2H), 3.57-3.42 (m, 1H), 3.40-3.22 (m, 1H), 1.24 (s, 9H).

A solution of Compound 45 and cesium fluoride (10 mmol, 1.5 g) in t-BuOH (10 mL) was heated at 70° C. for 2 hours. The reaction was then cooled to room temperature, diluted with ethyl acetate and the organic layer was washed with water and brine then dried (Na₂SO₄) and concentrated. Purification by column chromatography (silica gel, eluting with 10 to 20% ethyl acetate in hexanes) provided Compound 46 (0.94 g, 90% from 43). ¹H NMR (300 MHz, chloroform-d) δ=7.49 (m, 2H), 7.37 (m, 3H), 5.56 (s, 1H), 5.29-5.02 (m, 1H), 5.02-4.81 (m, 1H), 4.49-4.32 (m, 1H), 4.22-4.04 (m, 1H), 3.99-3.54 (m, 7H), 1.23 (s, 9H).

### c) Preparation of Compound 49

Potassium carbonate (3.2 mmol, 0.44 g) was added to a solution of compound 46 (1.18 mmol, 0.4 g) in methanol (10 mL). After stirring at room temperature for 3 hours, the solvent was evaporated under reduced pressure and the residue was partitioned between ethyl acetate and water. The organic layer was dried (Na₂SO₄) and concentrated to provide Compound 47 which was used without any further purification. ¹H NMR (300 MHz, chloroform-d) δ=7.58-7.30 (m, 5H), 5.54 (s, 1H), 5.23-4.94 (m, 1H), 4.39 (dd, J=4.7, 10.0 Hz, 1H), 4.02-3.43 (m, 6H), 2.25-2.08 (m, 1H).

Trifluoromethanesulfonic anhydride (0.45 mmol, 0.08 mL) was added to a cold (0° C.) solution of compound 47 (0.3 mmol, 0.08 g) and pyridine (0.05 mL). After stirring for one hour, the reaction was quenched by adding water and the organic layer was washed with water and brine then dried (Na₂SO₄) and concentrated to provide crude 49 which was used without any further purification. ¹H NMR (300 MHz, chloroform-d) δ=7.58-7.32 (m, 5H), 5.55 (s, 1H), 5.28 (1H, d, J=55 Hz), 5.02-4.85 (m, 1H), 4.42 (dd, J=4.9, 10.4 Hz, 1H), 4.09 (dd, J=5.7, 10.8 Hz, 1H), 4.01-3.80 (m, 2H), 3.78-3.50 (m, 2H); MS (e/z), 387 (m+1).

### Example 15

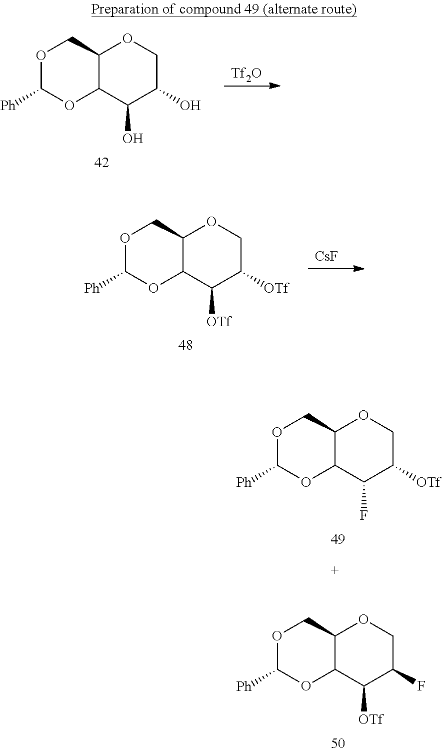

### a) Preparation of Compound 48

Trifluoromethanesulfonic anhydride (12.0 mmol, 2.0 mL) was added to a cold (0° C.) dichloromethane solution (40 mL) of Compound 42 (4.0 mmol, 1.0 g) and pyridine (16 mmol., 1.3 mL). After stirring for one hour, the reaction was quenched by adding water and the organic layer was washed with water and brine then dried and concentrated to provide crude Compound 48 (2.24 g, quantitative) which was used without any further purification. ¹H NMR (CDCl₃): δ 7.52-7.45 (m, 2H), 7.41-7.35 (m, 3H), 5.58 (s, 1H), 5.08 (1H, t, J=9 Hz), 5.06-4.91 (m, 1H), 4.50-4.25 (m, 2H), 3.83-3.69 (m, 2H), 3.65-3.43 (m, 2H). MS (e/z), 517 (m+1).

### b) Preparation of Compounds 49 and 50

Compound 48 (2.05 mmol, 1.1 g) and CsF (6.2 mmol., 0.94 g) were mixed with dry t-butanol (15 mL) and the mixture was stirred at 90° C. for 25 minutes. The reaction was cooled to room temperature and extracted with ethyl acetate. The ethyl acetate solution was concentrated to dryness and the residue was purified by silica gel chromatography by eluting with 5% ethyl acetate in hexanes. Compound 49 was obtained as clear oil (0.47 g, 59% yield). ¹H NMR (300 MHz, chloroform-d) δ=7.58-7.32 (m, 5H), 5.55 (s, 1H), 5.28 (1H, d, J=55 Hz), 5.02-4.85 (m, 1H), 4.42 (dd, J=4.9, 10.4 Hz, 1H), 4.09 (dd, J=5.7, 10.8 Hz, 1H), 4.01-3.80 (m, 2H), 3.78-3.50 (m, 2H); MS (e/z), 387 (m+1). Compound 50 was obtained as a white solid (0.14 g, 18% yield). ¹H NMR (CDCl₃): δ 7.50-7.43 (m, 2H), 7.40-7.34 (m, 3H), 5.64 (s, 1H), 5.15-4.90 (m, 2H), 4.45-4.15 (m, 3H), 3.80-3.52 (m, 2H), 3.55-3.40 (m, 1H). MS (e/z), 387 (m+1).

### Example 16

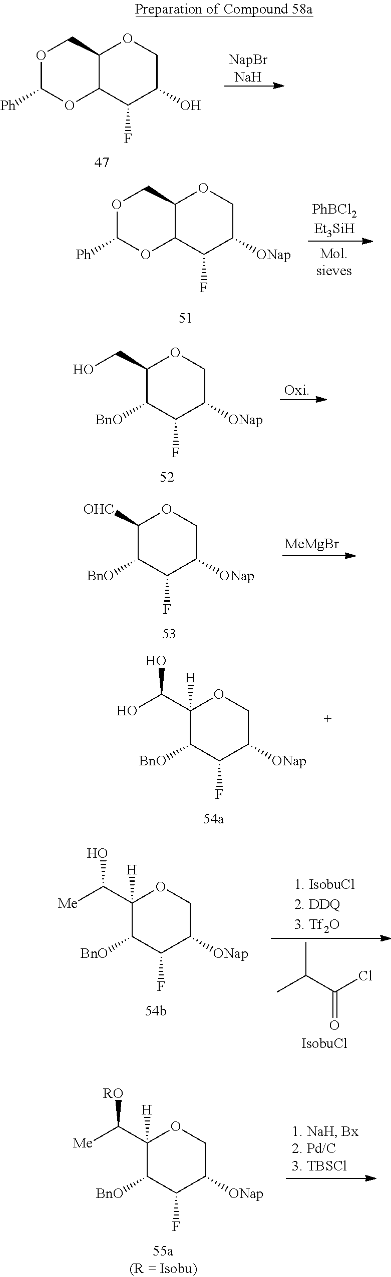

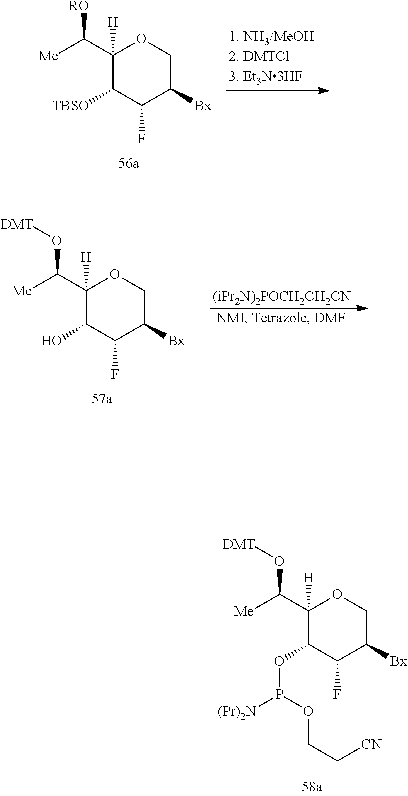

### a) Preparation of Compound 51

NaH (1.3 mmol, 52 mg) was added to a cold (0° C.) solution of Compound 47 (1.0 mmol, 0.27 g) and 2-(bromomethyl)naphthalene (1.3 mmol, 0.28 g) in dimethylformamide (5 mL). After stirring for one hour, the reaction was quenched by adding water and the mixture was extracted with ethyl acetate. The ethyl acetate solution was washed with water and brine then dried and concentrated to provide crude Compound 51 which was purified by silica gel column chromatography by eluting with 5% ethyl acetate in hexanes. Compound 51 was obtained as a white solid (0.4 g, quantitative). ¹H NMR (CDCl₃): δ 8.0-7.25 (m, 12H), 5.47 (s, 1H), 5.17 (1H, d, J=54 Hz), 4.87-4.76 (m, 2H), 4.40-4.30 (m, 1H), 3.95-3.78 (m, 2H), 3.75-3.56 (m, 2H), 3.51-3.39 (m, 2H). MS (e/z), 395, 417 (m+l, m+23).

### b) Preparation of Compound 52

Molecular sieves 4A (powder, 4.45 g) were placed in a 100 mL flask with heating at 140° C. over four hours with vacuation. After cooling to room temperature, Compound 51 and dichloro-methane (15 mL) were added. After stirring for one hour at room temperature, the mixture was cooled to −78° C., and Et₃SiH (4.11 mmol. 0.66 mL) and PhBCl₃ (3.63 mmol. 0.48 mL) were added successively with constant stirring. The mixture was stirred for an additional 10 minutes at −78° C. and 30% H₂O₂ (12.6 mmol. 1.6 mL) was added. After filtration, the reaction mixture was extracted with dichloromethane. The organic solution was washed with water and brine then dried and concentrated to provide crude Compound 52 which was purified by silica gel column chromatograph by eluting with 1% acetone in dichloromethane. Compound 52 was obtained as a white solid (0.31 g, 62%). ¹H NMR (CDCl₃): δ 7.87-7.77 (m, 4H), 7.52-7.46 (m, 3H), 7.40-7.30 (m, 5H), 5.14 (1H, d, J=54 Hz), 4.83-4.52 (m, 4H), 3.90-3.83 (m, 2H), 3.73-3.66 (m, 3H), 3.56-3.34 (m, 2H), 1.68 (1H, t, J=6 Hz). MS (e/z), 419 (m+23).

### c) Preparation of Compound 53

Compound 52 (0.025 mmol. 0.01 g) was dissolved in dichloromethane (0.3 mL), Dess-Martin reagent (0.025 mmol. 0.01 g) was added. The reaction was stirred at room temperature for 10 minute and concentrated to provide Compound 53. ¹H NMR (CDCl₃): δ 9.70 (s, 1H), 8.1-7.3 (m, 12H), 5.17 (1H, d, J=54 Hz), 4.80 (s, 2H), 4.45-4.75 (m, 2H), 4.25-4.20 (m, 1H), 4.0-3.90 (m, 1H), 3.85-3.35 (m, 3H).

### d) Preparation of Compound 58a

Compounds 54a and 54b are prepared from Compound 53 by adding MeMgBr in the presence of Cerium chloride. Alternately, compounds 54a and 54b can be interconverted to each other by means of a Mitsunobu reaction. The secondary hydroxyl group in 54a is protected as an ester, preferably as an isobutyryl ester and the 2′O-naphthyl group is removed using DDQ followed by reaction with triflic anhydride to provide Compound 55a. Reaction with a suitably protected nucleobase and a strong base such as sodium hydride in a solvent such as DMSO at temperatures between 50 and 100° C., followed by removal of the benzyl group using catalytic hydrogenation and reprotection as the silyl ether provides Compound 56a. Removal of the isobutyryl group using methanolic ammonia or potassium carbonate in methanol followed by reaction with DMTCl and lutidine and pyridine as the solvent at temperatures between 25 and 50 degree Celsius followed by removal of the silyl protecting group using triethylamine trihydrofluoride provides Compound 57a. A phosphitylation reaction provides the phosphoramidite, Compound 58a.

### Example 17

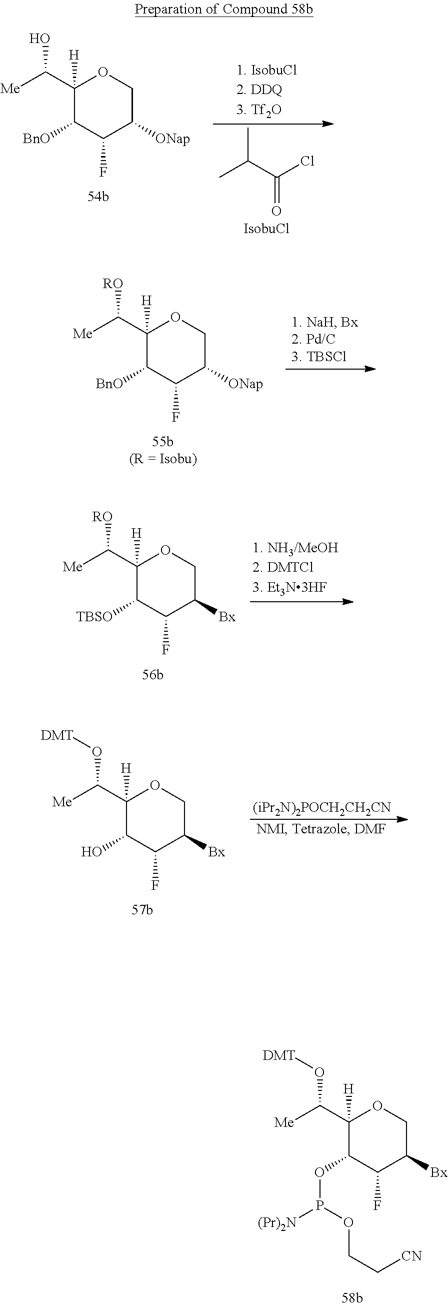

Compounds 54a and 54b are prepared from aldehyde 53 by adding MeMgBr in the presence of Cerium chloride. Alternately, compounds 54a and 54b can be interconverted to each other by means of a Mitsunobu reaction. The secondary hydroxyl group in 54b is protected as an ester, preferably as an isobutyryl ester and the 2′O-naphthyl group is removed using DDQ followed by reaction with triflic anhydride to provide Compound 55b. Reaction with a suitably protected nucleobase and a strong base such as sodium hydride in a solvent such as DMSO at temperatures between 50 and 100° C., followed by removal of the benzyl group using catalytic hydrogenation and reprotection as the silyl ether provides Compound 56b. Removal of the isobutyryl group using methanolic ammonia or potassium carbonate in methanol followed by reaction with DMTCl and lutidine and pyridine as the solvent at temperatures between 25 and 50 degree Celsius followed by removal of the silyl protecting group using triethylamine trihydrofluoride provides Compound 57b. A phosphitylation reaction provides phosphoramidite 58b.

### Example 18

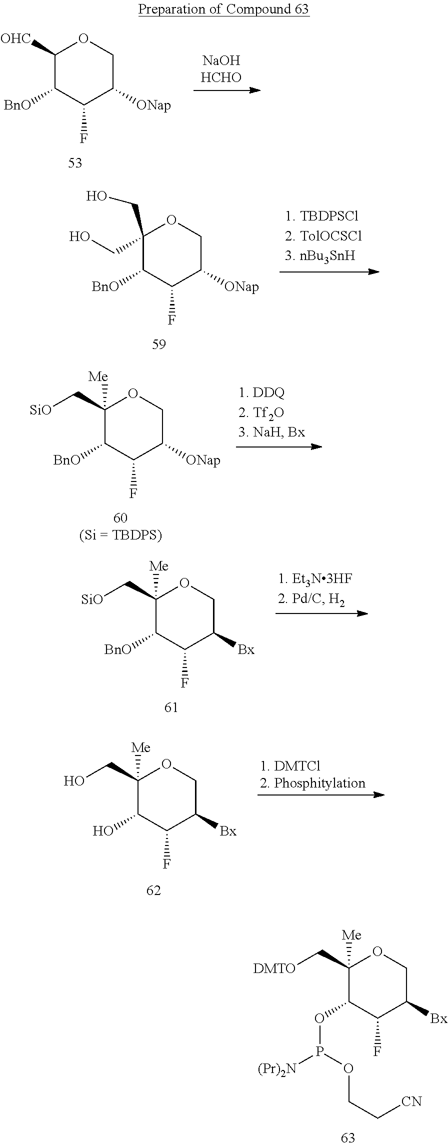

### a) Preparation of Compound 59

Compound 53 (0.7 mmol. 0.27 g) was dissolved in THF (2 mL), water (0.7 mL), HCHO (0.7 mL), and 4 N NaOH (aq., 0.7 mL) was added. The reaction was stirred at room temperature for three days. The reaction was extracted with ethyl acetate and washed with water and brine then dried and concentrated to provide crude 59 which was purified by silica gel column chromatograph by eluting with 10% acetone in dichloromethane. Compound 59 was obtained as a white solid (0.19 g, 64%). ¹H NMR (CDCl₃): 7.94-7.80 (m, 4H), 7.61-7.45 (m, 3H), 7.42-7.21 (m, 5H), 5.20 (1H, d, J-54 Hz), 4.49-4.40 (m, 4H), 4.20-3.35 (m, 11H), 2.10-1.95 (m, 1H), 1.90-1.75 (m, 1 H).

### b) Preparation of Compound 63

Reaction of Compound 59 with TBDPSCl provides a mixture of mono silylated products which are separated and the hydroxyl group is deoxygenated by means of a Barton deoxygenation reaction to provide Compound 60. Removal of the 2′β-naphthyl group with DDQ followed by triflation and reaction with a suitably protected nucleobase and a strong base such as sodium hydride in a solvent such as DMSO at temperatures between 50 and 100° C. provides Compound 61. Removal of the silyl protecting group using triethylamine trihydrofluoride followed by removal of the benzyl group by catalytic hydrogenation provides Compound 62. Protection of the primary hydroxyl group as the DMT ether followed by a phosphitylation reaction provides the phosphoramidite, Compound 63.

### Example 19

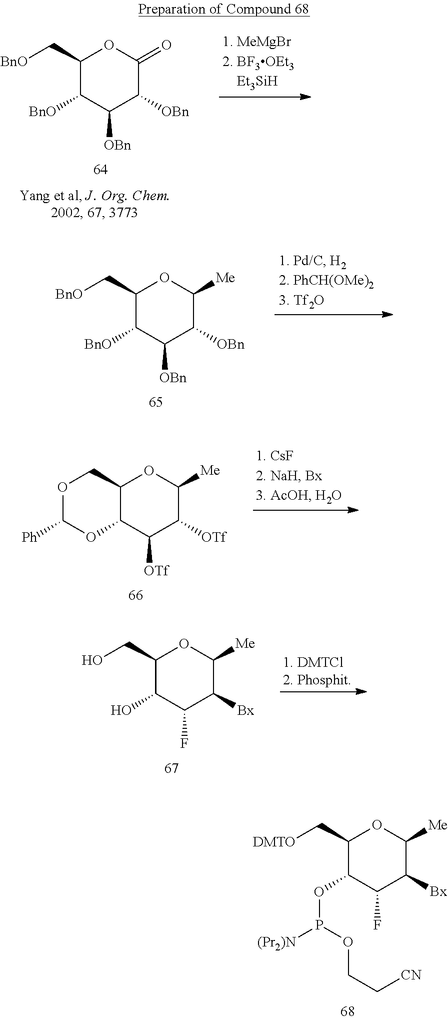

Compound 65 is prepared from known Compound 64 according to the method described by Bihovsky (J. Org. Chem., 1988, 53, 4026-4031). The benzyl protecting groups are removed using catalytic hydrogenation followed by protection of the 4′-OH and the 6′-OH as the benzylidene acetal. Reaction with triflic anhydride provides the bis triflate 66. Selective displacement of the 3′-triflate group using CsF as described in Example 15, followed by heating with a suitably protected nucleobase in the presence of a strong base like sodium hydride and a polar solvent like dimethyl-sulfoxide at temperatures between 50 and 100 degree Celsius and removal of the benzylidene protecting group using aqueous acetic acid at temperatures between 50 to 100 degree Celsius provides the nucleoside 67. Reaction of the primary alcohol with DMTCl followed by a phos-phitylation reaction provides the phosphoramidite, Compound 68.

### Example 20

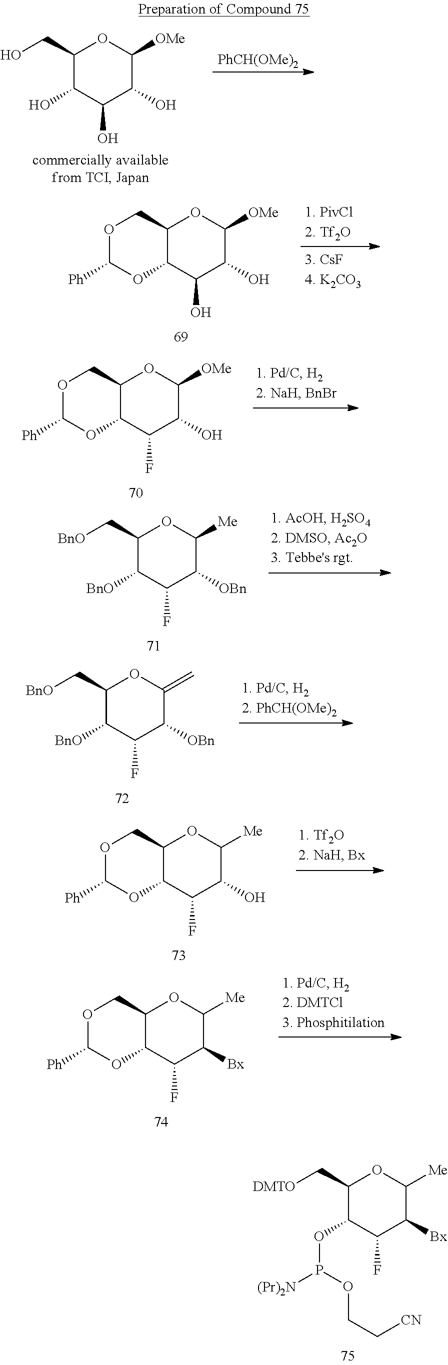

Compound 69 is prepared by reacting commercially available Methyl-β-D-glucopyranose with dimethylbenzylidene acetal in the presence of p-toluenesulfonic acid at temperatures between 60 and 80 degree Celsius. Selective protection of Compound 69 with pivaloyl chloride, triflation, displacement with CsF and hydrolysis of the pivaloyl ester with potassium carbonate in methanol as described in Example 14 provides Compound 70. Removal of the benzylidene protecting group followed by reprotection of the hydroxyl groups as the benzyl ether provides Compound 71. Hydrolysis of the OMe acetal by heating with acetic acid and aqueous sulfuric acid followed by oxidation of the lactol with acetic anhydride in DMSO and an olefination reaction with Tebbe's or Petassis's reagent provides the olefin 72. Reduction of the vinyl group and removal of the benzyl protecting groups using catalytic hydrogenation followed by reprotection of the 4′OH and the 6′OH as the benzylidene acetal provides Compound 73. Triflation with triflic anhydride followed by reaction with a suitably protected nucleobase and a strong base such as sodium hydride in a solvent such as DMSO at temperatures between 50 and 100° C. provides Compound 74. Removal of the benzylidene protecting group using catalytic hydrogenation, protection of the primary alcohol as the DMT ether and a phosphitylation reaction provides the phosphoramidite Compound 75.

### Example 21

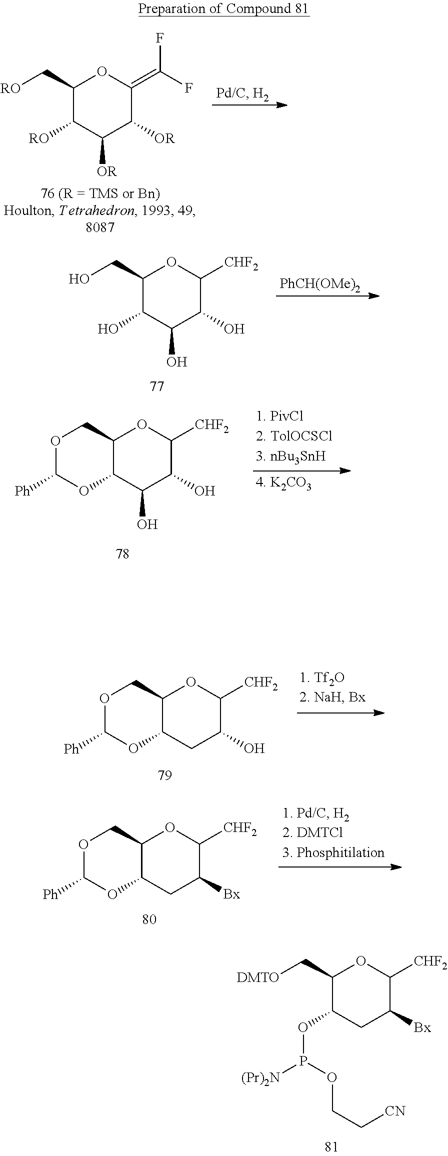

Compound 76 is prepared according to the procedure described by Houlton (Tetrahedron, 1993, 49, 8087) and is reduced to Compound 77 by means of a catalytic hydrogenation reaction. Protection of the 4′OH and the 6′OH as the benzylidene acetal provides Compound 78. Treatment of the 2′OH with pivaloyl chloride according to method described in Example 14 followed by Barton deoxygenation of the 3′OH group and hydrolysis of the pivaloyl ester provides Compound 79. Triflation with triflic anhydride followed by reaction with a suitably protected nucleobase and a strong base such as sodium hydride in a solvent such as DMSO at temperatures between 50 and 100° C. provides Compound 80. Removal of the benzylidene protecting group using catalytic hydrogenation, protection of the primary alcohol as the DMT ether and a phosphitylation reaction provides the phosphoramidite, Compound 81.

### Example 22

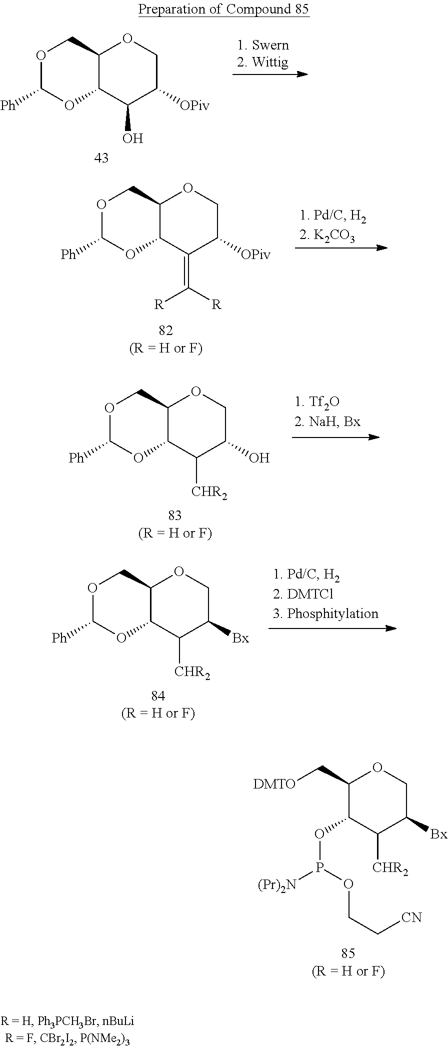

Oxidation of Compound 43 (prepared as per the procedures illustrated in Example 14) followed by a Wittig reaction provides Compound 82. Reduction of the olefin by means of a catalytic hydrogenation reaction followed by removal of the pivaloyl group with potassium carbonate in methanol provides Compound 83. Triflation with triflic anhydride followed by reaction with a suitably protected nucleobase and a strong base such as sodium hydride in a solvent such as DMSO at temperatures between 50 and 100° C. provides Compound 84. Removal of the benzylidene protecting group using catalytic hydrogenation, protection of the primary alcohol as the DMT ether and a phosphitylation reaction provides the phosphoramidite, Compound 85.

### Example 23

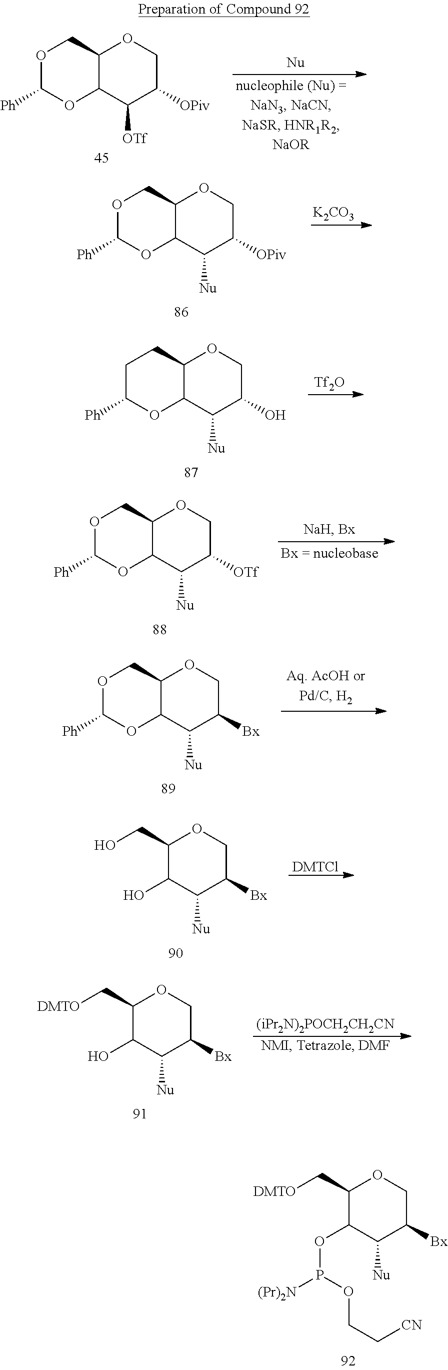

Compound 45 (prepared as per the procedures illustrated in Example 14) is reacted with a suitable nucleophile such as sodium azide, sodium cyanide, sodium sulfide, a primary or secondary amine derivative or sodium methoxide provides Compound 86 wherein the nucleophile (Nu) can be selected from any desired nucleophile which can include such nucleophiles as azide, cyanide, thiol, thioether, amine or alkoxide. Hydrolysis of the pivaloyl group using potassium carbonate provides Compound 87. Triflation of the hydroxyl group using triflic anhydride provides Compound 88. Reaction with a suitably protected nucleobase and a strong base such as sodium hydride in a solvent such as DMSO at temperatures between 50 and 100° C. provides Compound 89. Removal of the benzylidene protecting group using catalytic hydrogenation or by heating with aqueous acetic acid provides Compound 90. Protection of the primary alcohol as the DMT ether provides Compound 91 followed by a phosphitylation reaction provides the phosphoramidite, Compound 92.

### Example 24

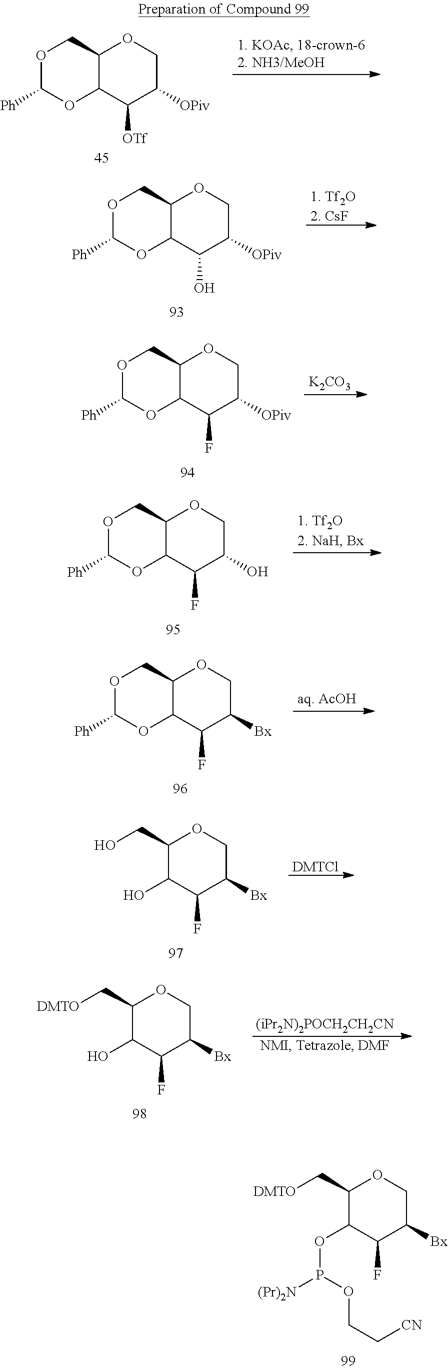

Compound 45 is treated with potassium acetate and 18-crown-6 in an appropriate solvent to afford S_{N}2 substitution of the triflate. The resulting product is treated with methanolic ammonia at reduced temperature to afford Compound 93. Alternately, Compound 45 can be subjected to Mitsunobu conditions (R₃P, DIAD, pO₂NBzOH), followed by aminolysis, to afford the same Compound 93. Sequential treatment of 93 with triflic anhydride, isolation of the triflate, and treatment with cesium fluoride in t-butyl alcohol gives 94, analogous to the preparation of Compound 46 from Compound 45 described above. Treatment of 94 with potassium carbonate in methanol generates the fluoro alcohol 95, which is converted to the triflate upon treatment with triflic anhydride in pyridine. Isolation, followed by treatment with a nucleobase in the presence of a strong base such as sodium hydride gives Compound 96. Removal of the benzylidene protecting group with 90% aqueous acetic acid gives Compound 97. Reaction with 4,4′-dimethoxytrityl chloride in pyridine gives Compound 98, which, following isolation, is converted to the cyanoethyl phosphoramidite, Compound 99.

### Example 25

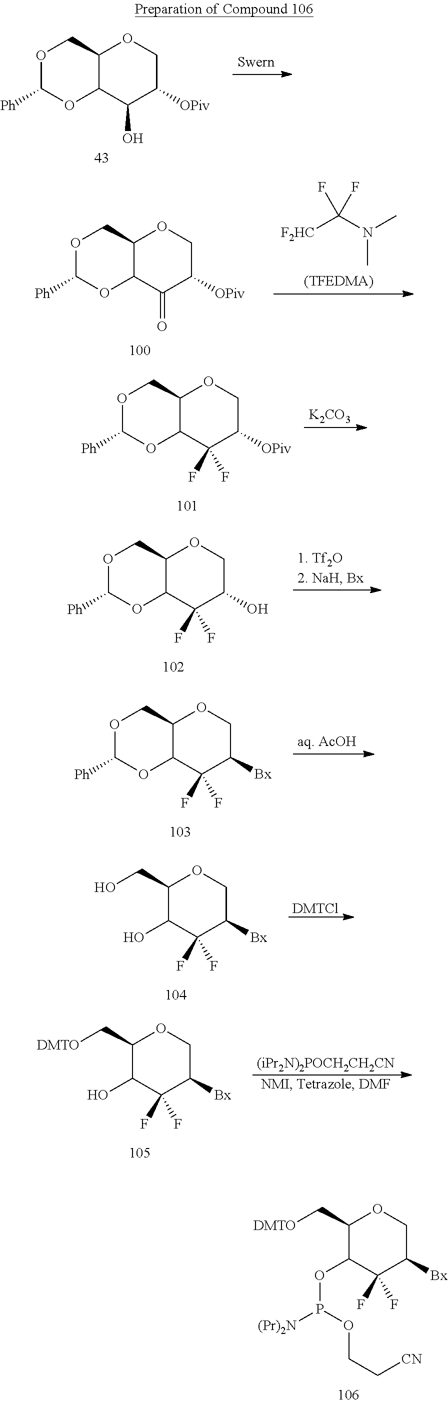

Oxidation of Compound 43 (prepared as per the procedures illustrated in Example 14) under Swern conditions (oxalyl chloride, DMSO, triethylamine, dichloromethane) gives ketone 100. Treatment with a fluorinating reagent such as 1,1,2,2-tetrafluoroethyl-N,N-dimethylamine (alternately deoxofluor or DAST) gives Compound 101. Removal of the pivaloyl group under potassium carbonate/methanol conditions gives Compound 102. Sequential treatment with triflic anhydride in pyridine, isolation, and treatment with a nucleobase in the presence of base gives the nucleoside analog, Compound 103. Removal of the benzylidene with 90% aqueous acetic acid gives Compound 104, which is converted to Compound 105 upon treatment with 4,4-dimethoxytrityl chloride in pyridine. A phosphitylation reaction provides the phosphoramidite, Compound 106.

### Example 26

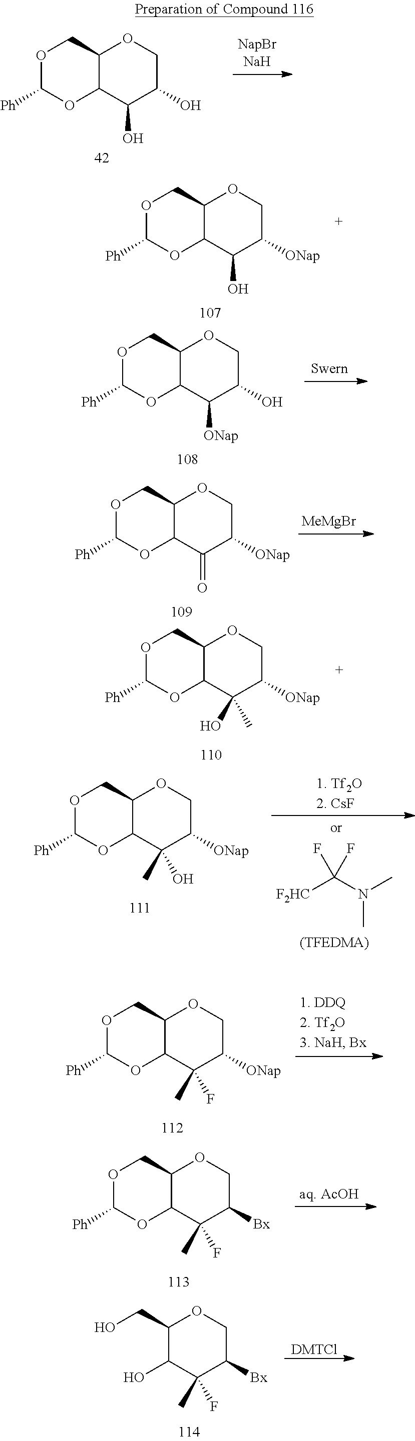

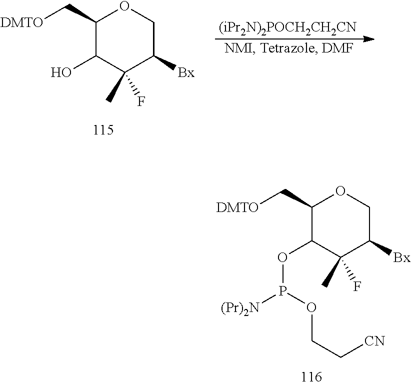

Treatment of Compound 42 (prepared as per the procedures illustrated in Example 14) with 2-(bromomethyl)-naphthalene (Nap bromide) in the presence of sodium hydride gives a mixture of Nap-protected regioisomers (107 and 108). Separation by silica gel chromatography provides the isomer, Compound 107. Oxidation of Compound 107 under Swern conditions (oxalyl chloride, DMSO, triethylamine, dichloromethane) gives the ketone, Compound 109, which is subsequently treated with methyl magnesium bromide (Methyl Grignard) to give a mixture of the methyl alcohols, compounds 110 and 111. Isolation of the desired stereoisomer 110 by silica gel chromatography, followed by formation of the triflate under triflic anhydride/pyridine conditions and treatment with cesium fluoride gives the fluorinated Compound 112. Alternatively, treatment of 110 with TFEDMA gives Compound 112 in a single process. Removal of the Nap protecting group with DDQ, followed by triflation, isolation, and treatment with a nucleobase in the presence of a base gives Compound 113. Removal of the benzylidene with 90% aqueous acetic acid affords Compound 114, which is converted to Compound 115 upon treatment with 4,4-dimethoxytrityl chloride in pyridine. A phosphitylation reaction provides the phosphoramidite, Compound 116.

### Example 27

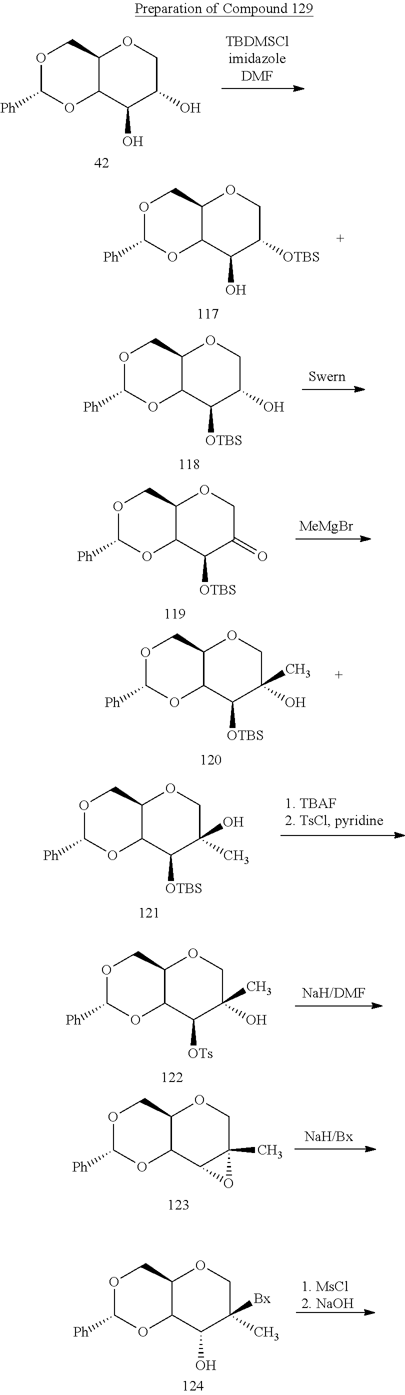

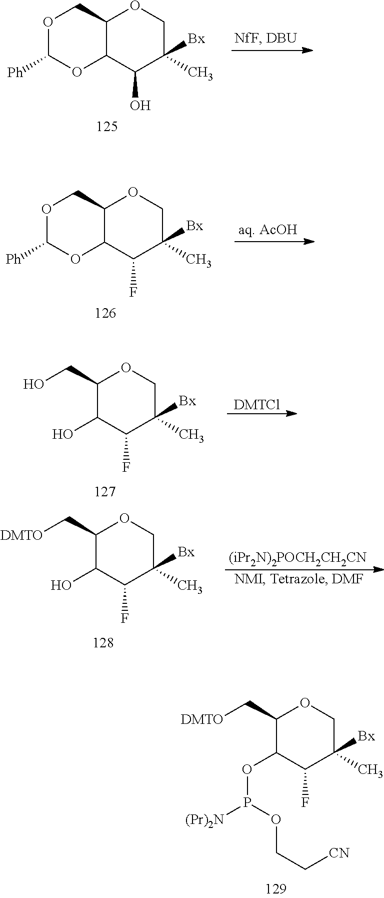

Treatment of Compound 42 (prepared as per the procedures illustrated in Example 14) with tertbutyldimethylsilyl chloride in the presence of imidazole and DMF yields a mixture of the silylated compounds 117 and 118 as described previously in Nucleosides, Nucleotides, and Nucleic Acids (2004), 23(1&2), 439-455. Following silica gel chromatography, the isomer, Compound 117 is oxidized under Swern conditions (oxalyl chloride, DMSO, triethylamine, dichloromethane) to generate the ketone, Compound 119. Treatment with methyl magnesium bromide gives a mixture of alcohols, compounds 120 and 121. Separation by silica gel chromatography, treatment of isolated Compound 120 with tetrabutylammonium fluoride, followed by conversion to the tosylate under tosyl chloride and pyridine conditions, gives Compound 122. Treatment with base converts tosylate 122 to the corresponding epoxide, Compound 123, as documented with similar compounds (Bioorg. Med. Chem. Lett. 1996, 6, 1457). Reaction of Compound 123 with a selected pyrimidine heterocycle (heterocyclic base) in the presence of base results in formation of Compound 124. Inversion of stereochemistry of the hydroxyl group is achieved by treatment with mesyl chloride, followed by hydrolysis of the resulting mesylate, which proceeds through an anhydro cyclic intermediate. Fluorination with nonafluorobutane sulfonyl fluoride under DBU/THF conditions gives the fluorinated Compound 126. Removal of the benzylidene group with 90% aqueous acetic acid affords Compound 127, which is converted to Compound 128 upon treatment with 4,4-dimethoxytrityl chloride in pyridine. A phosphitylation reaction provides the phosphoramidite, Compound 129.

### Example 28

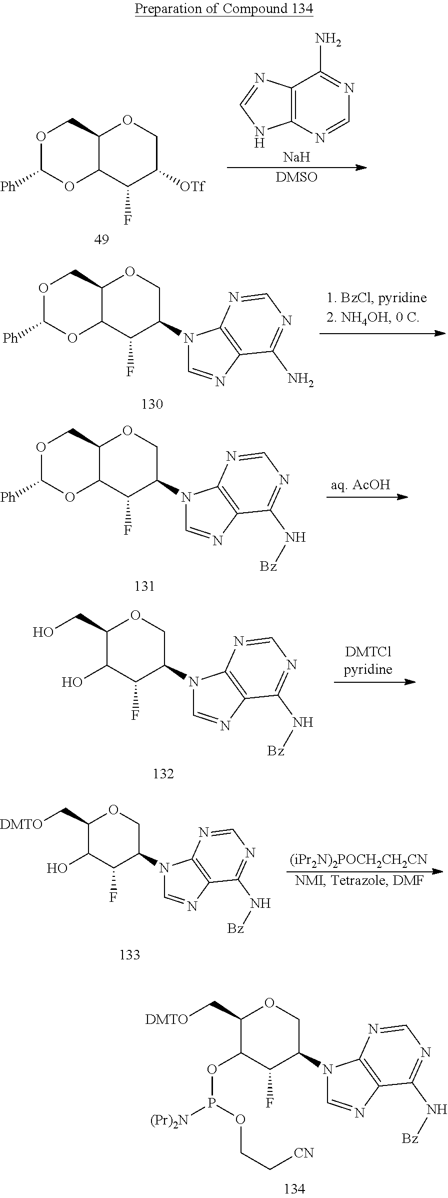

### a) Preparation of Compound 130

Compound 49 (prepared as per the procedures illustrated in Example 14, 10.8 mmol, 4.20 g) and adenine (54.5 mmol, 7.35 g) were suspended in anhydrous DMSO (80 mL). To this suspension was added sodium hydride (54.4 mmol, 2.18 g of a 60% mineral oil suspension). The resulting mixture was heated to 55° C. for 12 hours, cooled to room temperature and poured into water (400 mL). The mixture was extracted with ethyl acetate (3×400 mL), and the combined organic extracts were washed with half-saturated aqueous NaCl (3×500 mL). The organic layer was dried over anhydrous Na₂SO₄, filtered, and evaporated to give 3.93 g (97% yield) of a brown solid. NMR (¹H and ¹⁹F) and LCMS mass analysis were consistent with structure. This material was used without further purification.

### b) Preparation of Compound 131

Compound 130 (10.5 mmol, 3.93 g) was dissolved in anhydrous pyridine (50 mL). After cooling to 0° C., the solution was treated with benzoyl chloride (16.9 mmol, 1.97 mL). Stirring was continued at 0° C. for 15 minutes at which time the mixture was warmed to room temperature over 2.5 hours. The mixture was cooled to 0° C., quenched with 20 mL H₂O and stirred for 15 minutes. Concentrated aqueous NH₄OH (20 mL) was added to the mixture with stirring for 30 minutes. The mixture was concentrated mixture in vacuo to approximately 40 mL and poured into ethyl acetate (500 mL). The mixture was washed with half-saturated aqueous NaCl (3×500 mL), dried over anhydrous Na₂SO₄, filtered, and evaporated to a light-brown foam. Purification by silica gel chromatography (1.5% methanol in dichloromethane) yielded 2.33 g of Compound 131 as a light brown foam. NMR (¹H and ¹⁹F) and LCMS analyses were consistent with structure.

### c) Preparation of Compound 132

Compound 131 (4.84 mmol, 2.30 g) was dissolved in 70 mL of 90% (v/v) aqueous acetic acid. The solution was heated to 80° C. for 4 hours and then concentrated in vacuo to a viscous yellow oil. Triethylamine (10 drops) were added followed by 5 mL of methanol and 100 mL ethyl acetate. A white precipitate formed, which was collected by filtration, washed with ethyl acetate, and vacuum dried overnight. Final mass of white solid, Compound 132, was 1.28 g (69%). NMR (¹H and ¹⁹F) and LCMS analyses were consistent with structure of Compound 132.

### d) Preparation of Compound 133

Compound 132 (3.24 mmol, 1.25 g) was suspended in anhydrous pyridine (12 mL). The resulting suspension was cooled to 0° C. and treated with 4,4′-dimethoxytrityl chloride (5.19 mmol, 1.76 g) with stirring. Stirring was continued at 0° C. for 15 minutes and at room temperature for 5 hours when the mixture was quenched with methanol (2 mL) and concentrated in vacuo to a thick yellow oil. The oil was dissolved in dichloromethane (150 mL) and washed with saturated aqueous NaHCO₃ (100 mL) followed by saturated aqueous NaCl (2×100 mL). The organic layer was dried over anhydrous Na₂SO₄, filtered, and evaporated to a yellow foam. Purification by silica gel chromatography yielded 2.05 g (92% yield) of Compound 133 as a yellow foam. NMR analysis (¹H and ¹⁹F) was consistent with structure.

### e) Preparation of Compound 134

Compound 133 (2.59 mmol, 1.79 g) was dissolved in anhydrous DMF (6 mL) tetrazole (1.56 mmol, 109 mg), 1-methylimidazole (0.65 mmol, 52 μL) and tetraisopropylamino-2-cyanoethylphos-phorodiamidite (3.90 mmol, 1.24 mL) were added. After stirring for 4.5 hours, the reaction was quenched with the addition of triethylamine (10.4 mmol, 1.45 mL). The mixture was poured into ethyl acetate (150 mL), washed with saturated aqueous NaCl (4×100 mL), dried over anhydrous Na₂SO₄, filtered, and evaporated to a pale yellow foam. The solid was redissolved in ethyl acetate (7 mL) and precipitated by dropwise addition into 70 mL of hexanes. Silica gel purification (1:1 hexanes:ethyl acetate) of the resulting precipitate yielded 1.92 g (83%) of Compound 134 as a white foam. NMR (¹H, ¹⁹F, and ³¹P) are consistent with structure. ³¹P NMR (CDCl₃): δ ppm 151.64, 151.58, 150.37, 150.33.

### Example 29

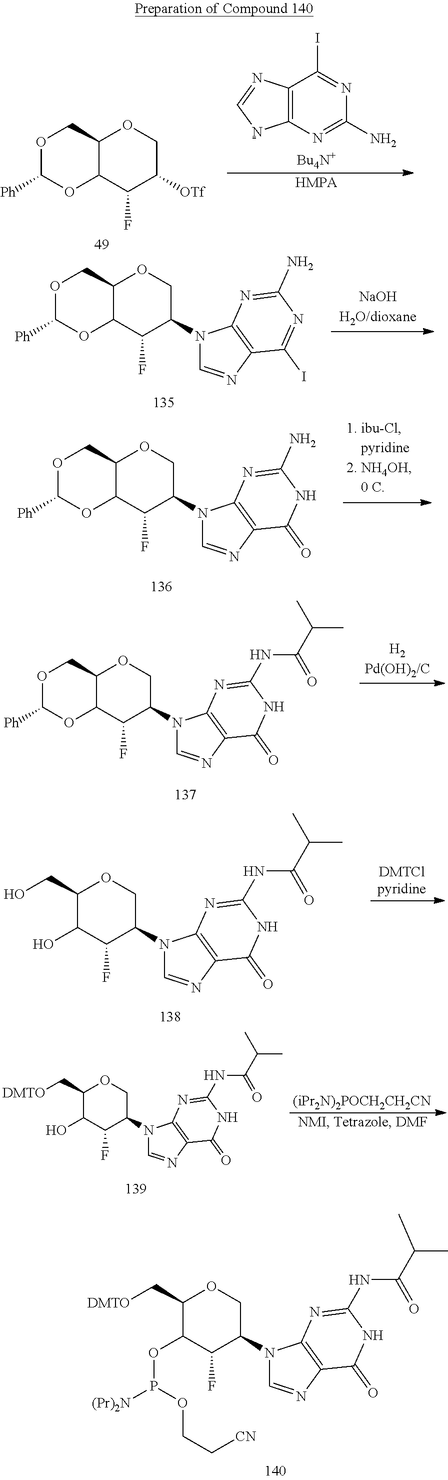

### a) Preparation of Compound 135

Compound 49 (prepared as per the procedures illustrated in Example 14, 7.51 mmol, 2.9 g) and 6-iodo-2-aminopurine tetrabutylammonium salt (17.6 mmol, 8.5 g, prepared as described in J. Org. Chem. 1995, 60, 2902-2905), were dissolved in anhydrous HMPA (26 mL). The mixture was stirred at room temperature for 18 hours, poured into ethyl acetate, washed with water and saturated NaCl, dried over anhydrous Na₂SO₄, filtered and evaporated. Purification by silica gel chromatography (1:1 hexanes:ethyl acetate) yielded 2.78 g (75% yield) of Compound 135. NMR (¹H and ¹⁹F) and LCMS analyses were consistent with structure.

### b) Preparation of Compound 136

Compound 135 (0.64 mmol, 0.32 g) was dissolved in 1,4-dioxane (9 mL) and 9 mL of 1M aqueous NaOH was added with heating at 55° C. for 18 hours. The mixture was cooled then neutralized with 1N HCl. The mixture was concentrated in vacuo and the residue purified by silica gel chromatography (5% methanol in dichloromethane) to yield 0.22 g (88% yield) of 136. NMR (¹H and ¹⁹F) and LCMS analyses were consistent with structure.

### c) Preparation of Compound 137

Compound 136 (3.23 mmol, 1.25 g) was dissolved in anhydrous pyridine (13.6 mL), cooled to 0° C., then treated with isobutyryl chloride (4.85 mmol, 0.51 mL). The mixture was warmed to room temperature and stirred for 6 hours. The mixture was cooled to 0° C. and treated with concentrated aqueous NH₄OH (3.2 mL) with stirring for 30 minutes. The mixture was poured into ethyl acetate (100 mL), washed with water (200 mL) and brine (200 mL), dried over anhydrous Na₂SO₄, filtered, and evaporated. Purification by silica gel chromatography (gradient of 0 to 5% methanol in dichloromethane) yielded 1.21 g (82% yield) of Compound 137. NMR (¹H and ¹⁹F) and LCMS analyses were consistent with structure.

### d) Preparation of Compound 138

Compound 137 (0.219 mmol, 0.103 g) was dissolved in methanol (10 mL) and acetic acid (0.2 mL) and Pd(OH)₂/C (0.44 g) were added with stirring under an atmosphere (balloon pressure) of hydrogen for 14 hours. The catalyst was removed by filtration, and the resulting filtrate was concentrated and triturated with acetonitrile to obtain Compound 138 as a white solid. NMR (¹H and ¹⁹F) and LCMS analyses were consistent with structure.

### e) Preparation of Compound 139

Compound 138 (3.83 mmol, 1.41 g) was dissolved in anhydrous pyridine (32 mL) and 4,4′-dimethoxytrityl chloride (5.0 mmol, 1.71 g) was added with stirring at room temperature for 3 hours followed by quenching with methanol (0.5 mL). The solution was concentrated in vacuo, then redissolved in ethyl acetate. The organic solution was washed with saturated aqueous NaHCO₃ and brine, dried over anhydrous Na₂SO₄, filtered, and evaporated. Purification by silica gel chromato-graphy yielded 1.63 g (70% yield) of 139. NMR (¹H and ¹⁹F) analysis was consistent with structure.

### f) Preparation of Compound 140

Compound 139 (1.59 mmol, 1.07 g) was dissolved in anhydrous DMF (4.25 mL) and tetrazole (1.35 mmol, 95 mg), 1-methylimidazole (0.45 mmol, 35 μL), and tetraisopropyl-2-cyanoethylphosphorodiamidite (2.25 mmol, 0.71 mL) were added. The mixture was stirred at room temperature for 3 hours, poured into ethyl acetate and washed with saturated aqueous NaHCO₃ and brine. The organic layer was dried over anhydrous Na₂SO₄, filtered, and evaporated. Purification by silica gel chromatography yielded 1.07 g (78% yield) of Compound 140. NMR (¹H, ¹⁹F, and ³¹P) analysis was consistent with structure. ³¹P NMR (CDCl₃): δ ppm 151.30, 151.24, 148.82, 148.78.

### Example 30

### Preparation of Gapped Oligomeric Compounds

Automated solid-phase synthesis was used to prepare oligomeric compounds used herein. One illustrative gapped oligomeric compound is ISIS-410131, having SEQ ID NO: 01, and Formula: 5′-C_{f}U_{f}TAGCACTGGCC_{f}U_{f}-3′. Each internucleoside linking group is a phosphorothioate, each of the T, A, G and C letters not followed by a subscript f designates a 13-D-2′-deoxyribonucleoside and each C_{f} and U_{f} is a monomer subunit wherein Bx is the heterocyclic base cytosine or uridine respectively and wherein the monomer subunit has the Formula and configuration:

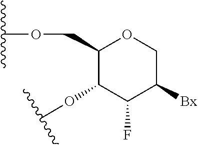

The synthesis of 410131 was carried out on a 40 μmol scale using an ÄKTA Oligopilot 10 (GE Healthcare) synthesizer with a polystyrene solid support loaded at 200 μmol/g with a universal linker. All nucleoside phosphoramidites, including compounds 8 and 13 were prepared as 0.1 M solutions in anhydrous acetonitrile. Coupling was performed using 4 molar equivalents of the respective phosphoramidite in the presence of 4,5-dicyanoimidazole, with a coupling time of 14 minutes. Thiolation of trivalent phosphorous to the phosphorothioate was achieved upon treatment with 0.2 M phenylacetyl disulfide in 1:1 3-picoline:acetonitrile. The resulting gapped oligomeric compound was deprotected using 1:1 triethylamine:acetonitrile (1 hour at room temperature), followed by conc. aq. NH₄OH at 55° C. for 7 hours. Ion exchange purification followed by reverse-phase desalting yielded 9.8 μmol (44 mg) of purified oligonucleotide. Mass and purity analysis by LC/MS ion-pair chromatography showed a UV purity of 98.5%, with an ESI mass of 4522.8 Da (calc. 4523.6 Da).

### Example 31

### 2-10-2 Gapped Oligomeric Compounds Targeted to PTEN: In Vitro Study

Gapped oligomeric compounds were synthesized and tested for their ability to reduce PTEN expression over a range of doses. bEND cells were transfected with gapped oligomeric compounds at doses of 0.3125, 0.625, 1.25, 2.5, 5, 10, 20 or 40 nM using 3 μg/mL Lipofectin in OptiMEM for 4 hrs, after which transfection mixtures were replaced with normal growth media (DMEM, high glucose, 10% FBS, pen-strep). RNA was harvested the following day (approximately 24 hours from the start of transfection) and analyzed for PTEN and cyclophilin A RNA levels using real time RT-PCR. Values represent averages and standard deviations (n=3) of PTEN RNA levels normalized to those of cyclophilin A.

The resulting dose-response curves were used to determine the IC₅₀s listed below. Tms were determined in 100 mM phosphate buffer, 0.1 mM EDTA, pH 7, at 260 nm using 4 μM of the modified oligomers listed below and 4 μM of the complementary RNA AGGCCAGUGCUAAG (SEQ ID NO: 7).

| SEQ ID NO./ | Composition | Tm | IC₅₀ |
|---|---|---|---|
| ISIS NO. | (5′ to 3′) | (° C.) | (nM) |
| 01/392753 | CₑUₑTAGCACTGGCCₑUₑ | 51.3 | 37 |
| 01/410312 | CₘUₘTAGCACTGGCCₘUₘ | 49.2 | 23 |
| 01/410131 | C_{f}U_{f}TAGCACTGGCC_{f}U_{f} | 50.0 | 16 |

Each internucleoside linking group is a phosphorothioate. Subscripted nucleosides are defined below wherein Bx is a heterocyclic base:

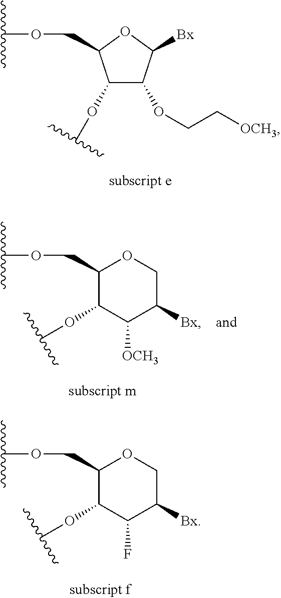

### Example 32

### 2-10-2 Gapped Oligomeric Compounds Targeted to PTEN: In Vitro Study

Gapped oligomeric compounds were synthesized and tested for their ability to reduce PTEN expression over a range of doses. bEND cells were transfected with gapped oligomeric compounds at doses of 0.3125, 0.625, 1.25, 2.5, 5, 10, 20 or 40 nM using 3 μg/mL Lipofectin in OptiMEM for 4 hrs, after which transfection mixtures were replaced with normal growth media (DMEM, high glucose, 10% FBS, pen-strep). RNA was harvested the following day (approximately 24 hours from start of transfection) and analyzed for PTEN and cyclophilin A RNA levels using real time RT-PCR. Values represent averages and standard deviations (n=3) of PTEN RNA levels normalized to those of cyclophilin A.

| SEQ ID NO./ | | | | | | | | |
|---|---|---|---|---|---|---|---|---|
| ISIS NO. | Composition (5′ to 3′) | | | | | | | |
| 02/392063 | ^{Me}CₗTₗTAGCACTGGC^{Me}CₗTₗ | | | | | | | |
| 01/410131 | C_{f}U_{f}TAGCACTGGCC_{f}U_{f} | | | | | | | |
| 02/417999 | ^{Me}C_{f}T_{f}TAGCACTGGC^{Me}C_{f}T_{f} | | | | | | | |
| SEQ ID NO./ | % UTC @ Dosage | | | | | | | |
| ISIS NO. | 0.3125 | 0.625 | 1.25 | 2.5 | 5 | 10 | 20 | 40 |
| 02/392063 | 86 | 83 | 66 | 40 | 36 | 24 | 32 | 17 |
| 01/410131 | 78 | 70 | 71 | 50 | 52 | 35 | 29 | 17 |
| 02/417999 | 98 | 108 | 77 | 72 | 68 | 43 | 33 | 20 |

Each internucleoside linking group is a phosphorothioate and superscript Me indicates that the following C is a 5-methyl C. Subscripted nucleosides are defined below wherein Bx is a heterocyclic base:

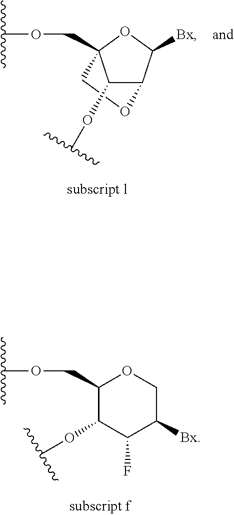

### Example 33

### 2-10-2 Gapped Oligomeric Compounds Targeted to PTEN: In Vivo Study

Six week old Balb/c mice (Jackson Laboratory, Bar Harbor, Me.) were injected once with the gapped oligomeric compounds targeted to PTEN at a dose of 20 or 60 mg/kg. The mice were sacrificed 72 hrs following administration. Liver tissues were homogenized and mRNA levels were quantitated using real-time PCR as described herein for comparison to untreated control levels (% UTC). Plasma chemistry analysis was completed.

| SEQ ID NO./ | Composition | dose | |
|---|---|---|---|
| ISIS NO. | (5′ to 3′) | (mg/kg) | % UTC |
| | saline | N/A | 100 |
| 01/392753 | CₑUₑTAGCACTGGCCₑUₑ | 20 | 84 |
| 01/392753 | CₑUₑTAGCACTGGCCₑUₑ | 60 | 68 |
| 01/410312 | CₘUₘTAGCACTGGCCₘUₘ | 20 | 83 |
| 01/410312 | CₘUₘTAGCACTGGCCₘUₘ | 60 | 27 |
| 01/410131 | C_{f}U_{f}TAGCACTGGCC_{f}U_{f} | 20 | 26 |
| 01/410131 | C_{f}U_{f}TAGCACTGGCC_{f}U_{f} | 60 | 8 |

Each internucleoside linking group is a phosphorothioate. Subscripted nucleosides are defined below:

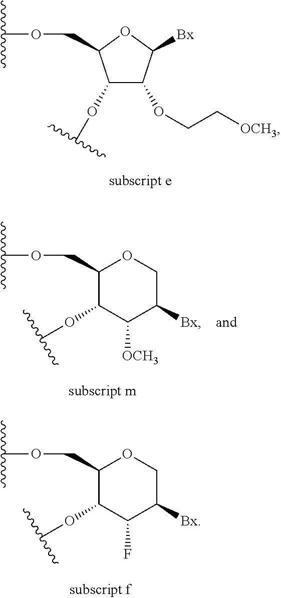

No increase in ALT and no significant effect on body or organ weights were observed after treatment with these gapped oligomeric compounds.

### Example 34

### Gapped Oligomeric Compounds Targeted to PTEN: In Vivo Study

Six week old Balb/c mice (Jackson Laboratory, Bar Harbor, Me.) were injected twice per week for three weeks with the gapped oligomeric compounds targeted to PTEN at a dose of 0.47, 1.5, 4.7 or 15 mg/kg. The mice were sacrificed 48 hours following last administration. Liver tissues were homogenized and mRNA levels were quantitated using real-time PCR as described herein for comparison to untreated control levels (% UTC). Plasma chemistry analysis was completed. Tms were determined in 100 mM phosphate buffer, 0.1 mM EDTA, pH 7, at 260 nm using 4 μM of the modified oligomers listed below and 4 μM of the complementary RNA AGGCCAGUGCUAAG (SEQ ID NO: 7).

| SEQ ID NO./ | Composition | Tm |
|---|---|---|
| ISIS NO. | (5′ to 3′) | (° C.) |
| 01/410131 | C_{f}U_{f}TAGCACTGGCC_{f}U_{f} | 50.7 |
| 02/417999 | ^{Me}C_{f}T_{f}TAGCACTGGC^{Me}C_{f}T_{f} | 52.6 |

Each internucleoside linking group is a phosphorothioate, superscript Me indicates that the following C is a 5-methyl C and nucleosides followed by a subscript f are defined in the formula below wherein Bx is a heterocyclic base:

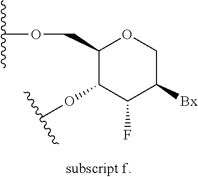

| SEQ ID NO./ | % UTC @ | % UTC @ | % UTC @ | % UTC @ |
|---|---|---|---|---|
| ISIS NO. | 0.47 mg/kg | 1.5 mg/kg | 4.7 mg/kg | 15 mg/kg |
| 01/410131 | — | — | — | 12 |
| 02/417999 | 77 | 64 | 31 | 10 |

| | |
|---|---|
| Saline | % UTC = 100 (dosage N/A) |

Liver transaminase levels, alanine aminotranferease (ALT) and aspartate aminotransferase (AST), in serum were also measured relative to saline injected mice. The approximate liver transaminase levels are listed in the table below.

| SEQ ID NO./ | AST @ | AST @ | AST @ | AST @ |
|---|---|---|---|---|
| ISIS NO. | 0.47 mg/kg | 1.5 mg/kg | 4.7 mg/kg | 15 mg/kg |
| 01/410131 | — | — | — | 106 |
| 02/417999 | 51 | 90 | 86 | 37 |
| Saline | 82 | | | |
| | (dosage N/A) | | | |
| 01/410131 | — | — | — | 27 |
| 02/417999 | 28 | 31 | 42 | 21 |
| Saline | 34 | | | |
| | (dosage N/A). | | | |

### Example 35

### Gapped Oligomeric Compounds Targeted to PTEN: In Vivo Study

Six week old Balb/c mice (Jackson Laboratory, Bar Harbor, Me.) were injected once with the gapped oligomeric compounds targeted to PTEN at a dose of 3.2, 10, 32 or 100 mg/kg. The mice were sacrificed 72 hours following administration. Liver tissues were homogenized and mRNA levels were quantitated using real-time PCR as described herein for comparison to untreated control levels (% UTC). Plasma chemistry analysis was completed. Tms were determined in 100 mM phosphate buffer, 0.1 mM EDTA, pH 7, at 260 nm using 4 μM of the modified oligomers listed below and 4 μM of the complementary RNA UCAAGGCCAGUGCUAAGAGU (SEQ ID NO: 8) for 2/14/2 motif oligomers and AGGCCAGUGCUAAG (SEQ ID NO: 7) for 2/10/2 oligomers.

| SEQ ID NO./ | Composition | | |
|---|---|---|---|
| ISIS NO. | (5′ to 3′) | Tm (° C.) | Motif |
| 03/411026 | C_{f}U_{f}GCTAGCCTCTGGATU_{f}U_{f} | 57.1 | 2/14/2 |
| 04/418000 | ^{Me}C_{f}T_{f}GCTAGCCTCTGGATT_{f}T_{f} | 58.5 | 2/14/2 5-CH₃ wings |
| 01/410131 | C_{f}U_{f}TAGCACTGGCC_{f}U_{f} | 50.7 | 2/10/2 |
| 02/417999 | ^{Me}C_{f}T_{f}TAGCACTGGC^{Me}C_{f}T_{f} | 52.6 | 2/10/2 5-CH₃ wings |
| 02/392063 | ^{Me}CₗTₗTAGCACTGGC^{Me}CₗTₗ | 60.5 | 2/10/2 5-CH₃ wings |

Each internucleoside linking group is a phosphorothioate and superscript Me indicates that the following C is a 5-methyl C. Subscripted nucleosides are defined below wherein Bx is a heterocyclic base:

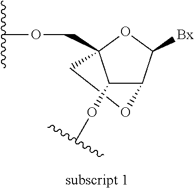

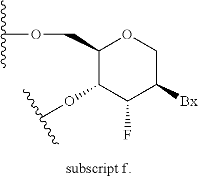

| SEQ ID NO./ | % UTC @ | % UTC @ | % UTC @ | % UTC @ |
|---|---|---|---|---|
| ISIS NO. | 3.2 mg/kg | 10 mg/kg | 32 mg/kg | 100 mg/kg |
| 02/392063 | 92 | 29 | 7 | 7 |
| 03/411026 | 92 | 52 | 12 | 7 |
| 04/418000 | 100 | 38 | 12 | 5 |
| 01/410131 | 100 | 59 | 9 | 3 |
| 02/417999 | 94 | 31 | 10 | 5 |

| | |
|---|---|
| Saline | % UTC = 100 |

Liver transaminase levels, alanine aminotranferease (ALT) and aspartate aminotransferase (AST), in serum were also measured relative to saline injected mice. The approximate liver transaminase levels are listed in the table below.

| SEQ ID NO./ | AST @ | AST @ | AST @ | AST @ |
|---|---|---|---|---|
| ISIS NO. | 3.2 mg/kg | 10 mg/kg | 32 mg/kg | 100 mg/kg |
| 02/392063 | 57 | 86 | 81 | 27399 |
| 03/411026 | 166 | 78 | 69 | 130 |
| 04/418000 | 90 | 94 | 80 | 345 |
| 01/410131 | 48 | 87 | 187 | 51 |
| 02/417999 | 72 | 126 | 99 | 55 |
| 02/392063 | 9 | 13 | 10 | 18670 |
| 03/411026 | 25 | 20 | 26 | 115 |
| 04/418000 | 17 | 33 | 44 | 321 |
| 01/410131 | 14 | 15 | 22 | 11 |
| 02/417999 | 13 | 22 | 15 | 11. |

### Example 36

### Gapped Oligomeric Compounds Targeted to PTEN: In Vivo Study

Six week old Balb/c mice (Jackson Laboratory, Bar Harbor, Me.) were injected once with the gapped oligomeric compounds targeted to PTEN at a dose of 3.2, 10, 32 or 100 mg/kg. The mice were sacrificed 72 hours following last administration. Liver tissues were homogenized and mRNA levels were quantitated using real-time PCR as described herein for comparison to untreated control levels (% UTC). Estimated ED₅₀ concentrations for each oligomeric compound were calculated using Graphpad Prism as shown below.

| SEQ ID NO./ | Composition | ED₅₀ |
|---|---|---|
| ISIS NO. | (5′ to 3′) | (mg/kg) |
| 02/417999 | ^{Me}C_{f}T_{f}TAGCACTGGCM^{e}C_{f}T_{f} | 7.5 |
| 02/425857 | ^{Me}CₕTₕTAGCACTGGC^{Me}CₕTₕ | 14.5 |

Each internucleoside linking group is a phosphorothioate and superscript Me indicates that the following C is a 5-methyl C. Subscripted nucleosides are defined below wherein Bx is a heterocyclic base:

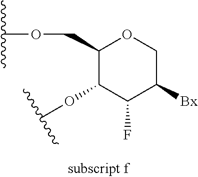

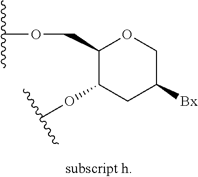

| SEQ ID NO./ | % UTC at dosage | | | |
|---|---|---|---|---|
| ISIS NO. | 3.2 mg/kg | 10 mg/kg | 32 mg/kg | 100 mg/kg |
| 02/417999 | 77 | 41 | 9 | 5 |
| 02/425857 | 76 | 72 | 20 | 6 |

| | |
|---|---|
| Saline | 100 |

Liver transaminase levels, alanine aminotranferease (ALT) and aspartate aminotransferase (AST), in serum were also measured relative to saline injected mice. The approximate liver transaminase levels are listed in the table below.

| SEQ ID NO./ | | | | |
|---|---|---|---|---|
| ISIS NO. | 3.2 mg/kg | 10 mg/kg | 32 mg/kg | 100 mg/kg |
| | AST (IU/L) at dosage | | | |
| 02/417999 | 72 | 126 | 99 | 55 |
| 02/425857 | 88 | 64 | 77 | 46 |
| Saline | 77 | | | |
| | (dosage: n/a) | | | |
| | ALT (IU/L) at dosage | | | |
| 02/417999 | 26 | 24 | 19 | 31 |
| 02/425857 | 28 | 26 | 29 | 51 |
| Saline | 31 | | | |
| | (dosage: n/a). | | | |

### Example 37

### Gapped Oligomeric Compounds

Oligomeric compounds were prepared having a gapped motif with various gap and wing sizes. Tms were determined in 100 mM phosphate buffer, 0.1 mM EDTA, pH 7, at 260 nm using 4 μM of the modified oligomers listed below and 4 μM of either the complementary RNA UCAAGGCCAGUGCUAAGAGU (SEQ ID NO: 8) for Tm¹ or AGGCCAGUGCUAAG (SEQ ID NO: 7) for Tm².

| SEQ ID NO./ | | | Gapmer |
|---|---|---|---|
| ISIS NO. | Composition (5′ to 3′) | Tm¹(° C.) | Design |
| 02/417999 | ^{Me}C_{f}T_{f}TAGCACTGGC^{Me}C_{f}T_{f} | 59.4 | 2-10-2 |
| 02/425858 | ^{Me}C_{f}T_{f}T_{f}AGCACTGG^{Me}C_{f}^{Me}C_{f}T_{f} | 67.4 | 3-8-3 |
| 05/425859 | T_{f}^{Me}C_{f}T_{f}TAGCACTGGC^{Me}C_{f}T_{f}T_{f} | 65.0 | 3-10-3 |
| 05/425860 | T_{f}^{Me}C_{f}T_{f}T_{f}AGCACTGG^{Me}C_{f}^{Me}C_{f}T_{f}T_{f} | 70.4 | 4-8-4 |
| 06/425861 | ^{Me}C_{f}T_{f}^{Me}C_{f}T_{f}T_{f}AGCACTGG^{Me}C_{f}^{Me}C_{f}T_{f}T_{f} | 74.3 | 5-8-4 |

Each internucleoside linking group is a phosphorothioate and superscript Me indicates that the following C is a 5-methyl C. Subscripted nucleoside is defined below wherein Bx is a heterocyclic base:

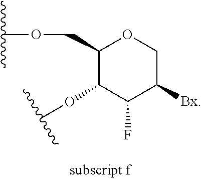

### Example 38

### Hemimers Targeted to PTEN: In Vivo Study

Six week old Balb/c mice (Jackson Laboratory, Bar Harbor, Me.) were injected once with the gapped oligomeric compounds targeted to PTEN at a dose of 1.6, 5, 16 or 50 mg/kg. The mice were sacrificed 72 hours following last administration. Liver tissues were homogenized and mRNA levels were quantitated using real-time PCR as described herein for comparison to untreated control levels (% UTC). Estimated ED₅₀ concentrations for each oligomeric compound were calculated using Graphpad Prism as shown below. Tms were determined in 100 mM phosphate buffer, 0.1 mM EDTA, pH 7, at 260 nm using 4 μM of the modified oligomers listed below and 4 μM of either the complementary RNA UCAAGGCCAGUGCUAAGAGU (SEQ ID NO: 8) for Tm¹ or AGGCCAGUGCUAAG (SEQ ID NO: 7) for Tm².

| SEQ ID NO./ | Composition | | |
|---|---|---|---|
| ISIS NO. | (5′ to 3′) | Tm¹ | Tm² |
| 02/412471 | ^{Me}CₗTₗTₗAGCACTGGC^{Me}CT | 65.5 | 62.5 |
| 02/429495 | ^{Me}C_{f}T_{f}T_{f}AGCACTGGC^{Me}CT | 63.8 | 59.6 |

Each internucleoside linking group is a phosphorothioate and superscript Me indicates that the following C is a 5-methyl C. Subscripted nucleosides are defined below wherein Bx is a heterocyclic base:

| 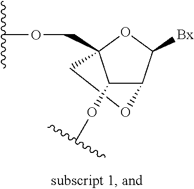 | | | | |
|---|---|---|---|---|
| 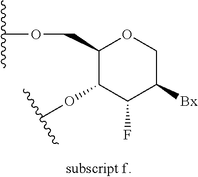 | | | | |
| SEQ ID NO./ | % UTC at dosage | | | |
| ISIS NO. | 1.6 mg/kg | 5 mg/kg | 16 mg/kg | 50 mg/kg |
| 02/412471 | 85 | 51 | 20 | 23 |
| 02/429495 | 90 | 79 | 40 | 17 |

| | |
|---|---|
| Saline | % UTC = 100 |

Liver transaminase levels, alanine aminotranferease (ALT) and aspartate aminotransferase (AST), in serum were also measured relative to saline injected mice. The approximate liver transaminase levels are listed in the table below.

| SEQ ID NO./ | | | | |
|---|---|---|---|---|
| ISIS NO. | 1.6 mg/kg | 5 mg/kg | 16 mg/kg | 50 mg/kg |
| | AST (IU/L) at dosage | | | |
| 02/412471 | 67 | 67 | 69 | 4572 |
| 02/429495 | 95 | 54 | 77 | 58 |
| Saline | 68 | | | |
| | (dosage: n/a) | | | |
| | ALT (IU/L) at dosage | | | |
| 02/412471 | 29 | 31 | 33 | 3419 |
| 02/429495 | 33 | 31 | 38 | 23 |
| Saline | 35 | | | |
| | (dosage: n/a). | | | |

### Example 39

### THP Containing Oligonucleotides for Modulating Splicing

Two oligonucleotides complementary to SMN1 were synthesized and melting temperatures were determined. The oligonucleotides comprised a gapmer motif having MOE wings and tetrahydropyran nucleosides in the gap.

| SEQ ID NO./ | | | |
|---|---|---|---|
| ISIS NO. | Composition (5′ to 3′) | Tm¹ (° C.) | Gapmer Design |
| 03/440758 | Tₑ^{Me}CₑA_{f}^{Me}C_{f}T_{f}T_{f}T_{f}^{Me}C_{f}A_{f}T_{f}A_{f}A_{f}T_{f}G_{f}^{Me}C_{f}T_{f}GₑGₑ | 75.55 | 2-14-2 |
| 04/440759 | TₑTₑT_{f}^{Me}C_{f}A_{f}T_{f}A_{f}A_{f}T_{f}G_{f}^{Me}C_{f}T_{f}G_{f}Gₑ^{Me}Cₑ | 72.63 | 2-11-2 |

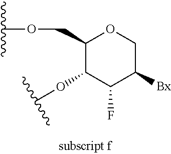

What is claimed is:

1-194. (canceled)

195. A compound comprising an oligomeric compound consisting of 12 to 30 linked monomers, wherein the oligomeric compound comprises at least 4 regions, wherein each monomer within each region comprises the same type of sugar moiety and wherein the sugar moieties of the monomers of adjacent regions are different from one another; and wherein:
at least one region comprises 2-20 linked monomers and each of the other regions independently comprises 1-20 linked monomers; and wherein
at least one region is a tetrahydropyran region, wherein each tetrahydropyran region independently comprises one or more tetrahydropyran nucleoside analog of Formula I: 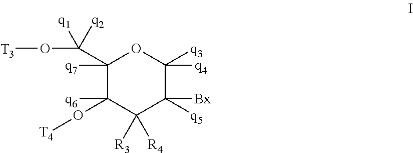
wherein independently for each of said tetrahydropyran nucleoside analogs of Formula I:
Bx is a heterocyclic base moiety;
T₃ and T₄ are each, independently, an internucleoside linking group linking the tetrahydropyran nucleoside analog to the oligomeric compound or one of T₃ and T₄ is an internucleoside linking group linking the tetrahydropyran nucleoside analog to the oligomeric compound and the other of T₃ and T₄ is H, a hydroxyl protecting group, a linked conjugate group or a 5′ or 3′-terminal group;
q₁, q₂, q₃, q₄, q₅, q₆ and q₇ are each independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;
R₃ and R₄ are each independently, H, hydroxyl, halogen, C₁-C₆ alkyl, substituted C₁-C₆ alkyl, C₁-C₆ alkoxy or substituted C₁-C₆ alkoxy;
each substituted group comprises one or more optionally protected substituent groups independently selected from halogen, OJ₁, NJ₁J₂, SJ₁, N₃, OC(═X)J₁, OC(═X)NJ₁J₂, NJ₃C(═X)NJ₁J₂ and CN, wherein X is O, S or NJ₁ and each J₁, J₂ and J₃ is, independently, H or C₁-C₆ alkyl; and
wherein the remaining regions are non-tetrahydropyran regions, wherein the monomers of each non-tetrahydropyran region are independently modified or unmodified nucleosides or nucleoside analogs other than tetrahydropyran nucleoside analogs.

196. The compound of claim 195 having at least two tetrahydropyran regions.

197. The compound of claim 195 having at least three tetrahydropyran regions.

198. The compound of claim 195 having a motif:
T₁-(Nu₁)ₙ₁-(Nu₂)ₙ₂-(Nu₃)ₙ₃-(Nu₄)ₙ₄-(Nu₅)ₙ₅-T₂, wherein:
Nu₁, Nu₃, and Nu₅ are each independently tetrahydropyran nucleoside analogs of Formula I;
Nu₂ and Nu₄ are each independently modified or unmodified nucleosides or nucleoside analogs other than tetrahydropyran nucleoside analogs;
each of n1 and n5 is, independently from 0 to 3;
the sum of n2 plus n4 is between 10 and 25;
n3 is from 0 and 5; and
each T₁ and T₂ is, independently, H, a hydroxyl protecting group, an optionally linked conjugate group or a capping group.

199. The compound of claim 195, wherein one of R₃ and R₄ is H and the other of R₃ and R₄ is H, OCH₃ or F for at least one tetrahydropyran nucleoside analog.

200. The compound of claim 195 wherein at least one internucleoside linking group is a phosphodiester internucleoside linking group.

201. The compound of claim 195 wherein at least one internucleoside linking group is a phosphorothioate internucleoside linking group.

202. The compound of claim 201 wherein each internucleoside linking group is a phosphorothioate internucleoside linking group.

203. A compound comprising an oligomeric compound consisting of 12 to 30 linked monomers, wherein the oligomeric compound comprises at least one monomer comprising a tetrahydropyran nucleoside analog of Formula I: 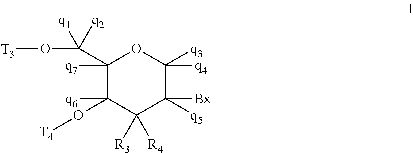
wherein independently for each of said tetrahydropyran nucleoside analogs of Formula I:
Bx is a heterocyclic base moiety;
T₃ and T₄ are each, independently, an internucleoside linking group linking the tetrahydropyran nucleoside analog to the oligomeric compound or one of T₃ and T₄ is an internucleoside linking group linking the tetrahydropyran nucleoside analog to the oligomeric compound and the other of T₃ and T₄ is H, a hydroxyl protecting group, a linked conjugate group or a 5′ or 3′-terminal group;
q₁, q₂, q₃, q₄, q₅, q₆ and q₇ are each independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;
R₃ and R₄ are each independently, H, hydroxyl, halogen, C₁-C₆ alkyl, substituted C₁-C₆ alkyl, C₁-C₆ alkoxy or substituted C₁-C₆ alkoxy;
each substituted group comprises one or more optionally protected substituent groups independently selected from halogen, OJ₁, NJ₁J₂, SJ₁, N₃, OC(═X)J₁, OC(═X)NJ₁J₂, NJ₃C(═X)NJ₁J₂ and CN, wherein X is O, S or NJ₁ and each J₁, J₂ and J₃ is, independently, H or C₁-C₆ alkyl; and
wherein the oligomeric compound has a nucleobase sequence, and wherein at least a portion of the nucleobase sequence of the oligomeric compound is complementary to a portion of a target non-coding RNA.

204. The compound of claim 203 wherein the target non-coding RNA is a target pri-microRNA, a target pre-microRNA, or a target microRNA.

205. The compound of claim 204 comprising at least two tetrahydropyran nucleosides of Formula I.

206. The compound of claim 205 wherein each monomer of the oligomeric compound is a tetrahydropyran nucleoside of Formula I.

207. The compound of claim 204 comprising a plurality of tetrahydropyran nucleosides of Formula I and a plurality of non-tetrahydropyran nucleosides wherein the tetrahydropyran nucleosides of Formula I and the non-tetrahydropyran nucleosides are arranged in an alternating motif.

208. A compound comprising an oligomeric compound consisting of 12 to 30 linked monomers, wherein the oligomeric compound comprises at least one monomer comprising a tetrahydropyran nucleoside analog of Formula I: 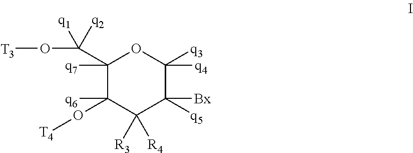
wherein independently for each of said tetrahydropyran nucleoside analogs of Formula I:
Bx is a heterocyclic base moiety;
T₃ and T₄ are each, independently, an internucleoside linking group linking the tetrahydropyran nucleoside analog to the oligomeric compound or one of T₃ and T₄ is an internucleoside linking group linking the tetrahydropyran nucleoside analog to the oligomeric compound and the other of T₃ and T₄ is H, a hydroxyl protecting group, a linked conjugate group or a 5′ or 3′-terminal group;
q₁, q₂, q₃, q₄, q₅, q₆ and q₇ are each independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;
R₃ and R₄ are each independently, H, hydroxyl, halogen, C₁-C₆ alkyl, substituted C₁-C₆ alkyl, C₁-C₆ alkoxy or substituted C₁-C₆ alkoxy;
each substituted group comprises one or more optionally protected substituent groups independently selected from halogen, OJ₁, NJ₁J₂, SJ₁, N₃, OC(═X)J₁, OC(═X)NJ₁J₂, NJ₃C(═X)NJ₁J₂ and CN, wherein X is O, S or NJ₁ and each J₁, J₂ and J₃ is, independently, H or C₁-C₆ alkyl; and
wherein the oligomeric compound has a nucleobase sequence, and wherein at least a portion of the nucleobase sequence of the oligomeric compound is complementary to a portion of a target pre-mRNA.

209. The compound of claim 208 wherein the target pre-mRNA encodes SMN2.

210. The compound of claim 208 comprising at least two tetrahydropyran nucleosides of Formula I.

211. The compound of claim 208 wherein each monomer of the oligomeric compound comprises a tetrahydropyran nucleoside of Formula I.

212. The compound of claim 208 comprising a plurality of tetrahydropyran nucleosides of Formula I and a plurality of non-tetrahydropyran nucleosides, wherein the tetrahydropyran nucleosides Formula I and the non-tetrahydropyran nucleosides are arranged in an alternating motif.

213. A method of modulating the amount or activity of a target non-coding RNA in a cell comprising contacting the cell with a compound of claim 203 and thereby modulating the amount or activity the target non-coding RNA in the cell.

214. A method of modulating processing of a target pre-mRNA in a cell comprising contacting the cell with an oligomeric compound according to claim 208 and thereby modulating the processing of the pre-mRNA in the cell.
